(19) United States
(12) Patent Application Publication
(10) Pub. No.: US 2009/0068224 A1
(43) Pub. Date: Mar. 12, 2009

(54) METHOD TO IDENTIFY POLYPEPTIDE TOLL-LIKE RECEPTOR (TLR) LIGANDS

(75) Inventors: Valerian Nakaar, Hamden, CT (US); Yan Huang, North Branford, CT (US); Thomas Powell, Madison, CT (US)

Correspondence Address: BAKER BOTTS L.L.P., 30 ROCKEFELLER PLAZA, 44th Floor, NEW YORK, NY, 10112-4498, US

(73) Assignee: VAXINNATE CORPORATION, Cranbury, NJ (US)

(21) Appl. No.: 11/815,095

(22) PCT Filed: Jan. 26, 2006
(86) PCT No.: PCT/US2006/002906

§ 371 Date: Oct. 14, 2008

Related U.S. Application Data
(60) Provisional application No. 60/648,723, filed on Jan. 31, 2005.

(51) Int. Cl. A61K 39/12; C40B 10/00; C40B 30/04; C07K 2/00; A61K 39/00; A61P 35/00; A61P 37/08; A61P 31/12; A61K 38/02; C12N 5/06; A61K 39/145

(52) U.S. Cl. 424/209.1; 506/1; 506/9; 530/350; 514/2; 435/375; 424/184.1; 424/277.1; 424/275.1; 424/218.1; 424/204.1

(57) ABSTRACT

The present invention provides novel methods to identify polypeptide ligands for Toll-like Receptors (TLRs), such as TLR2, TLR4 and TLR5. The method involves the use of phage display technology in an iterative biopanning procedure. The invention also provides polypeptide TLR ligands identified by the methods of the invention. In preferred embodiments, the polypeptide TLR ligands so identified modulate TLR signaling and thereby regulate the Innate Immune Response. The invention also provides vaccines comprising a polypeptide TLR ligand identified by the methods of the invention and an antigen. The invention also provides methods of modulating TLR signaling using the polypeptide TLR ligands and vaccines of the invention.

This application claims priority to U.S. Provisional Patent Application Ser. No. 60/648,723, filed on Jan. 31, 2005.

The research leading to this invention was supported, in part, by contract # HHSN266200400043C/N01-AI-40043 awarded by the National Institutes of Health. Accordingly, the United States government may have certain rights to this invention.

### FIELD OF THE INVENTION

The present invention provides novel methods to identify polypeptide ligands for Toll-like Receptors (TLRs), such as TLR2, TLR4 and TLR5. The method involves the use of phage display technology in an iterative biopanning procedure. The invention also provides polypeptide TLR ligands identified by the methods of the invention. In preferred embodiments, the polypeptide TLR ligands so identified modulate TLR signaling and thereby regulate the Innate Immune Response. The invention also provides vaccines comprising a polypeptide TLR ligand identified by the methods of the invention and an antigen. The invention also provides methods of modulating TLR signaling using the polypeptide TLR ligands and vaccines of the invention.

### BACKGROUND OF THE INVENTION

Multicellular organisms have developed two general systems of immunity to infectious agents. The two systems are innate or natural immunity (usually referred to as “innate immunity”) and adaptive (acquired) or specific immunity. The major difference between the two systems is the mechanism by which they recognize infectious agents. Recent studies have demonstrated that the innate immune system plays a crucial role in the control of initiation of the adaptive immune response and in the induction of appropriate cell effector responses (Fearon et al. Science 1996; 272:50-53 and Medzhitov et al. Cell 1997; 91:295-298).

The innate immune system uses a set of germline-encoded receptors for the recognition of conserved molecular patterns present in microorganisms. These molecular patterns occur in certain constituents of microorganisms including: lipopolysaccharides, peptidoglycans, lipoteichoic acids, phosphatidyl cholines, bacterial proteins, including lipoproteins, bacterial DNAs, viral single and double-stranded RNAs, unmethylated CpG-DNAs, mannans, and a variety of other bacterial and fungal cell wall components. Such molecular patterns can also occur in other molecules such as plant alkaloids. These targets of innate immune recognition are called Pathogen Associated Molecular Patterns (PAMPs) since they are produced by microorganisms and not by the infected host organism (Janeway et al. Cold Spring Harb. Symp. Quant. Biol. 1989; 54:1-13 and Medzhitov et al. Curr. Opin Imnunol. 1997; 94:4-9). PAMPs are discrete molecular structures that are shared by a large group of microorganisms. They are conserved products of microbial metabolism, which are not subject to antigenic variability (Medzhitov et al. Cur Op Immun 1997; 94:4-9).

The receptors of the innate immune system that recognize PAMPs are called Pattern Recognition Receptors (PRRs) (Janeway et al. Cold Spring Harb. Symp. Quant. Biol. 1989; 54: 1-13 and Medzhitov et al. Curr. Opin. Immunol. 1997; 94:4-9). These receptors vary in structure and belong to several different protein families. Some of these receptors recognize PAMPs directly (e.g., CD14, DEC205, collectins), while others (e.g., complement receptors) recognize the products generated by PAMP recognition.

Cellular PRRs are expressed on effector cells of the innate immune system, including cells that function as professional antigen-presenting cells (APC) in adaptive immunity. Such effector cells include, but are not limited to, macrophages, dendritic cells, B lymphocytes, and surface epithelia. This expression profile allows PRRs to directly induce innate effector mechanisms, and also to alert the host organism to the presence of infectious agents by inducing the expression of a set of endogenous signals, such as inflammatory cytokines and chemokines. This latter function allows efficient mobilization of effector forces to combat the invaders.

The best characterized class of cellular PRRs are members of the family of Toll-like Receptors (TLRs), so called because they are homologous to the Drosophila Toll protein which is involved both in dorsoventral patterning in Drosophila embryos and in the immune response in adult flies (Lemaitre et al. Cell 1996; 86:973-83). At least 12 mammalian TLRs, TLRs 1 through 11 and TLR13, have been identified to date (see, for example, Medzhitov et al. Nature 1997; 388:394-397; Rock et al Proc Natl Acad Sci USA 1998; 95:588-593; Takeuchi et al. Gene 1999; 231:59-65; and Chuang and Ulevitch. Biochim Biophys Acta. 2001; 1518:157-61).

In mammalian organisms, such TLRs have been shown to recognize PAMPs such as the bacterial products LPS (Schwandner et al. J. Biol. Chem. 1999; 274:17406-9 and Hoshino et al. J. Immunol. 1999; 162:3749-3752), lipoteichoic acid (Schwandner et al. J. Biol. Chem. 1999; 274:17406-9), peptidoglycan (Yoshimura et al. J. Immunol. 1999; 163:1-5), lipoprotein (Aliprantis et al. Science 1999; 285:736-9), CpG-DNA (Hemmi et al. Nature 2000; 408:740-745), and flagellin (Hayashi et al. Nature 2001; 410:1099-1103), as well as the viral product double-stranded RNA (Alexopoulou et al. Nature 2001; 413:732-738) and the yeast product zymosan (Underhill. J Endotoxin Res. 2003; 9:176-80).

TLR2 is essential for the recognition of a variety of PAMPs, including bacterial lipoproteins, peptidoglycan, and lipoteichoic acids. TLR3 is implicated in recognition of viral double-stranded RNA. TLR4 is predominantly activated by lipopolysaccharide. TLR5 detects bacterial flagellin and TLR9 is required for response to unmethylated CpG DNA. Recently, TLR7 and TLR8 have been shown to recognize small synthetic antiviral molecules (Jurk M. et al. Nat Immunol 2002; 3:499). Furthermore, in many instances, TLRs require the presence of a co-receptor to initiate the signaling cascade. One example is TLR4 which interacts with MD2 and CD14, a protein that exists both in soluble form and as a GPI-anchored protein, to induce NF-κB in response to LPS stimulation (Takeuchi and Akira. Microbes Infect 2002; 4:887-95). FIG. 1 illustrates some of the known interactions between PAMPs and TLRs (reviewed in Janeway and Medzhitov. Annu Rev Immunol 2002; 20:197-216).

TLR2 is involved in the recognition of, e.g., multiple products of Gram-positive bacteria, mycobacteria and yeast, including LPS and lipoproteins. TLR2 is known to heterodimerize with other TLRs, a property believed to extend the range of PAMPs that TLR2 can recognize. For example, TLR2 cooperates with TLR6 in the response to peptidoglycan (Ozinsky et al. Proc Natl Acad Sci USA 2000; 97:13766-71) and diacylated mycoplasmal lipopeptide (Takeuchi et al. Int Immunol 2001; 13:933-40), and associates with TLR^{hi} to recognize triacylated lipopeptides (Takeuchi et al. J Immunol 2002; 169:10-4). Pathogen recognition by TLR2 is strongly enhanced by CD14. A pentapeptide derived from fimbrial subunit protein, ALTTE, was shown to activate monocytes and epithelial cells via TLR2 signaling (Ogawa et al. FEMS Immunol Med Microbiol 1995; 11:197-206; Asai et al Infect Immun 2001; 69:7378-7395; and Ogawa et al. Eur J Immunol 2002; 32:2543-2550). A single amino acid substitution (A to G) in the peptide (GLTTE) was shown to antagonize the activity of the wild-type peptide and full-length protein (Ogawa et al. FEMS Inmiunol Med Microbiol 1995; 11: 197-206).

TLR4, the first human TLR identified, is the receptor for Gram-negative lipopolysaccharide (LPS). The TLR4 gene was shown to be mutated in C3H/HeJ and C57BL/10ScCr mice, both of which are low responders to lipopolysaccharide (LPS) (Poltorak et al. Science 1998; 282:2085-8). However, TLR4 alone is not sufficient to confer LPS responsiveness. TLR4 requires MD-2, a secreted molecule, to functionally interact with LPS (Shimazu et al. J Exp Med 1999; 189:1777-82). Furthermore, a third protein, called CD14, was shown to participate in LPS signaling, leading to NF-κB translocation. This signaling is mediated through the adaptor protein MyD88, but also through a MyD88-independent pathway that involves the (TIR) domain-containing adapter protein (TIRAP) (Horng et al. Nat Immunol 2001; 2:835-41).

TLR5 is the Toll-like Receptor that recognizes flagellin from both Gram-positive and Gram-negative bacteria. Activation of the receptor stimulates the production of proinflammatory cytokines, such as TNFα, through signaling via the adaptor protein MyD88 and the serine kinase IRAK (Gewirtz et al. J Immunol 2001; 167:1882-5 and Hayashi et al. Nature 2001; 410:1099-103). TLR5 can generate a proinflammatory signal as a homodimer, suggesting that it might be the only TLR required for flagellin recognition (Hayashi et al. Nature 2001; 410:1099-103).

Activation of signal transduction pathways by TLRs leads to the induction of various genes including inflammatory cytokines, chemokines, major histocompatability complex, and co-stimulatory molecules (e.g., B7). The intracellular signaling pathways initiated by activated TLRs vary slightly from TLR to TLR, with some signaling pathways being common to all TLRs (shared pathways), and some being specific to particular TLRs (specific pathways).

In one of the shared pathways, the cytoplasmic adaptor proteins myeloid differentiation factor 88 (MyD88) and TOLLIP (Toll-interacting protein) independently associate with the cytoplasmic tail of the TLR. Each of these adaptors recruits the serine/threonine kinase IRAK to the receptor complex, each with different kinetics. Recruitment of IRAK to the receptor complex results in auto-phosphorylation of IRAK. Phosphorylated IRAK then associates with another adaptor protein, TRAF6. TRAF6, in turn, associates with and activates the MAP kinase kinases TAK-1 and MKK6. Activation of TAK-1 leads, via one or more intermediate steps, to the activation of the IkB kinase (IKK), whose activity directs the degradation of IκB and the activation of NF-κB. Activation of MKK6 leads to the activation of JNK (c-Jun N-terminal kinase) and the MAP kinase p38 (Medzhitov and Janeway. Trends in Microbiology 2000; 8:452-456 and Medzhitov. Nature Reviews 2001; 1:135-145). Other cytoplasmic proteins implicated in TLR signaling include the RHO family GTPase RAC1 and protein kinase B (PKB), as well as the adapter protein TIRAP and its associated proteins protein kinase R (PKR) and the PKR regulatory proteins PACT and p58 (Medzhitov. Nature Reviews 2001; 1:135-145). Cytoplasmic proteins specifically implicated in TLR-signaling by mutational studies include MyD88 (Schnare et al. Nature Immunol 2001; 2:947-950), TIRAP (Horng et al. Nature Immunol 2001; 2:835-842), IRAK and TRAF6 (Medzhitov et al. Mol Cell 1998; 2:253-258), RICK/Rip2/CARDIAK (Kobayashi et al. Nature 2002; 416:194-199), IRAK-4 (Suzuki et al. Nature 2002; 416:750-746), and Mal (MyD88-adapter like) (Fitzgerald et al. Nature 2001; 413:78-83).

Due to TLR signaling through shared pathways (e.g. NF-κB, see above), some biological responses will likely be globally induced by any TLR signaling event. However, an emerging body of evidence demonstrates divergent responses induced by the specific pathways of individual TLRs. For example, TLR2 and TLR4 activate different immunological programs in human and murine cells, manifested in divergent patterns of cytokine expression (Hirschfeld et al. Infect Immun 2001; 69:1477-1482 and Re and Strominger. J Biol Chem. 2001; 276:37692-37699). These divergent phenotypes could be detected in an antigen-specific response, when lipopolysaccharides that signal through TLR2 or TLR4 were used to guide the response (Pulendran et al. J Immun 2001; 167:5067-5076). TLR4 and TLR2 signaling requires the adaptor TIRAP/Mal, which is involved in the MyD88-dependent pathway (Horng et al. Nature 2002; 420:329-33). TLR3 triggers the production of IFNβ in response to double-stranded RNA, in a MyD88-independent manner. This response is mediated by the adaptor TRIF/TICAM-1 (Yamamoto et al. J Immunol. 2002; 169:6668-72). TRAM/TICAM2 is another adaptor molecule involved in the MyD88-independent pathway (Miyake. Int Immunopharmacol. 2003; 3:119-28) which function is restricted to the TLR4 pathway (Yamamoto et al Nat Immunol. 2003; 4: 1144-50).

Thus, different TLR “switches” turn on different immune response “circuits”, where activation of a particular TLR determines the type of antigen-specific response that is triggered. Depending upon the cell type exposed to a PAMP and the particular TLR that binds to that PAMP, the profile of cytokines produced and secreted can vary. This variation in TLR signaling response can influence, for example, whether the resultant adaptive immune response will be predominantly T-cell- or B-cell-mediated, as well as the degree of inflammation accompanying the response.

As discussed above, the innate immune system plays a crucial role in the control of initiation of the adaptive immune response and in the induction of appropriate cell effector responses. Recent evidence demonstrates that fusing a polypeptide ligand specific for a Toll-like Receptor (TLR) to an antigen of interest generates a vaccine that is more potent and selective than the antigen alone. The inventors have previously shown that immunization with recombinant TLR-ligand:antigen fusion proteins: a) induces antigen-specific T-cell and B-cell responses comparable to those induced by the use of conventional adjuvant, b) results in significantly reduced non-specific inflammation; and c) results in CD8 T-cell-mediated protection that is specific for the fused antigen epitopes (see, for example, US published patent applications 2002/0061312 and 2003/0232055 to Medzhitov, and US published patent application 2003/0175287 to Medzhitov and Kopp, all incorporated herein by reference). Mice immunized with a fusion protein consisting of the polypeptide PAMP BLP linked to Leishmania major antigens mounted a Type 1 immune response characterized by antigen-induced production of γ-interferon and antigen-specific IgG₂ₐ (Cote-Sierra et al. Infect Immun 2002; 70:240-248). The response was protective, as demonstrated by experiments in which immunized mice developed smaller lesions than control mice did following challenge with live L. major.

Thus, the binding of PAMPs to TLRs activates immune pathways that can be mobilized for the development of more potent vaccines. Ideally, a vaccine design should ensure that every cell that is exposed to pathogen-derived antigen also receives a TLR receptor innate immune signal and vice versa. This can be effectively achieved by designing the vaccine to contain a chimeric macromolecule of antigen plus PAMP, e.g., a fusion protein of PAMP and antigen(s). Such molecules trigger signal transduction pathways in their target cells that result in the display of co-stimulatory molecules on the cell surface, as well as antigenic peptide in the context of major histocompatability complex molecules.

Although polypeptide ligands to some TLRs are known (see FIG. 1), cognate polypeptide ligands for other TLRs have not been discovered. Furthermore, for many of the known TLR ligands, the particular amino acid residues that contribute to ligand:TLR interaction are not known. Gross deletion studies, and alanine-scanning and site directed mutagenesis studies, have been used to delineate the critical amino acids in E. coli flagellin (fliC; Donelly and Steiner. J Biol Chem 2002; 277:40456-40461) and Measles Virus hemagglutinin (HA; Bieback et al. J Virol 2002; 76:8729-8736) necessary for PAMP activity. In these protocols, every polypeptide ligand variant construct must be individually expressed and the resulting recombinant protein purified for biological activity assays. Thus, these previously disclosed strategies for characterization of TLR polypeptide ligands are laborious and time-consuming.

A need exists in the art for methods to identify novel polypeptide ligands for TLRs. In particular, the need exists for the identification of polypeptide ligands specific for individual TLR receptors, which can be used to specifically tune the innate immune system response. The present invention fulfills these needs in the art by providing a method for identifying novel polypeptide ligands of TLRs based upon screening of phage display libraries for the ability to bind live cells expressing a TLR of interest. This “biopanning” procedure can be applied to identify novel peptides that interact specifically with individual TLRs. These polypeptide TLR ligands have the potential to be powerful and selective activators of the innate immune system, and may be engineered into vaccines to generate vigorous antigen-specific immune responses with minimal inflammation. Such TLR-specific polypeptide ligands can be incorporated into TLR-ligand:antigen conjugate vaccines, whereby the TLR-ligand will provide for an enhanced antigen-specific immune response as regulated by signaling through a particular TLR.

Furthermore, a need exists in the art for efficient methods to further characterize known polypeptide TLR ligands. The invention further provides methods to optimize the polypeptide sequence of known TLR ligands. These novel and optimized polypeptide TLR ligands may be incorporated into vaccines, e.g., for use against infectious diseases that pose a public health and national defense threat.

Phage display is a selection technique in which a peptide or protein is genetically fused to a coat protein of a bacteriophage (Smith. Science 1985; 228:1315-1317). The fusion protein is displayed on the exterior of the phage virion, while the DNA encoding the fusion protein resides within the virion. This physical linkage between the displayed protein and the DNA encoding it allows screening of vast numbers of variants of the protein by a simple in vitro selection procedure termed “biopanning”. Phage display technology offers a very powerful tool for the isolation of new ligands from large collections of potential ligands including short peptides, antibody fragments and randomly modified physiological ligands to receptors (Scott and Smith. Science 1990; 249:386-390; Smith and Scott. Meth Enz 1993; 217:228-257; and Smith and Petrenko. Chem. Rev 1997; 97:391-410). These systems have been effectively employed in studies of structural and functional aspects of receptor-ligand interactions using either purified receptors immobilized on a polymer surface (Smith and Petrenko. Chem. Rev 1997; 97:391-410), or the receptors in their natural environment on the surface of living cells (Fong. et al. Drug Dev Res 199433:64-70; Doorbar and Winter. J Mol Biol 1994; 244:361369; Goodson et al. Proc Natl Acad Sci USA 1994; 91:7129-7133; and Szardenings et al. J Biol Chem 1997; 272:27943-27948).

Cationic antimicrobial peptides (CAMPs) are relatively small (˜20-50 amino acids), cationic and amphipathic peptides of variable length, sequence and structure. These peptides contain a high percentage (20 to 60%) of the positively charged amino acids histidine, lysine and/or arginine. Several hundred CAMPs have been isolated from a wide variety of animals (both vertebrates and invertebrates), plants, bacteria and fungi. These peptides have been obtained from many different cellular sources, e.g. macrophages, neutrophils, epithelial cells, haemocytes, fat bodies, and the reproductive tract. CAMPs form part of the innate immune response of a wide variety of animal species, including insects, amphibians and mammals. In humans CAMPs, such as defensins, cathelicidins and thrombocidins, protect the skin and epithelia against invading microorganisms and assist neutrophils and platelets in host defense. To our knowledge, none of the reported CAMPs is a ligand for a TLR.

### SUMMARY OF THE INVENTION

The invention is directed to a method to identify a polypeptide TLR ligand comprising: a) providing a multiplicity of test phage in the form of a phage display library, wherein each individual test phage comprises a nucleic acid insert encoding a test polypeptide; b) contacting a TLR^{lo} cell with the multiplicity of test phage; c) retaining the test phage that do not bind to the TLR^{lo} cell; d) contacting a TLR^{hi} cell, wherein the TLR is the same TLR as in step b), with the test phage retained in step c); e) retaining the test phage that bind to the TLR^{hi} cell; f) amplifying the test phage retained in step e); g) optionally, repeating steps a) through f); and h) characterizing the polypeptide encoded by the nucleic acid insert of a test phage amplified in step f), wherein the polypeptide characterized in step h) is a polypeptide TLR ligand. In particularly preferred embodiments, the steps a) through f) are performed at least 4 times.

In preferred embodiments, the TLR is a mammalian TLR. In preferred embodiments, the TLR is TLR2, TLR4, or TLR5.

In preferred embodiments, the TLR^{lo} cell and the TLR^{hi} cell are the same cell type. In particularly preferred embodiments, the TLR^{lo} cell and the TLR^{hi} cell are both a HEK293 cell, or both an NIH3T3 cell. In preferred embodiments, the TLR^{lo} cell and the TLR^{hi} cell are both a mammalian cell.

In some embodiments, step h) comprises: i) determining the nucleic acid sequence of the nucleic acid insert; and ii) using the nucleic acid sequence from step i) to deduce the amino acid sequence of the polypeptide encoded by the nucleic acid insert.

In some embodiments, step h) comprises: i) translating the nucleic acid insert to generate the polypeptide encoded by the nucleic acid insert; and ii) characterizing said polypeptide. In particular embodiments, step ii) comprises determining the amino acid sequence of the polypeptide. In particular embodiments, step ii) comprises confirming the ability of the polypeptide to modulate TLR signaling.

The invention is further directed to a polypeptide TLR ligand identified by the methods of the invention.

The invention is also directed to a polypeptide comprising: i) a polypeptide TLR ligand identified by the methods of the invention; and ii) at least one antigen. In certain embodiments, the antigen is a polypeptide antigen. In certain embodiments, the antigen is a pathogen-related antigen, a tumor-associated antigen, or an allergen-related antigen. In particularly preferred embodiments, the pathogen-related antigen is an Influenza antigen, a Listeria monocytogenes antigen, or a West Nile Virus antigen.

The invention is also directed to a vaccine comprising one of the aforementioned polypeptides of the invention.

The invention is further directed to a vaccine comprising: i) a polypeptide TLR ligand identified by the methods of the invention; ii) at least one antigen; and iii) optionally, a pharmaceutically acceptable carrier. In preferred embodiments, the polypeptide TLR ligand and the antigen are covalently linked. In preferred embodiments, the at least one antigen is a polypeptide antigen. In certain embodiments, the antigen is a pathogen-related antigen, a tumor-associated antigen, or an allergen-related antigen. In certain embodiments, the pathogen-related antigen is an Influenza antigen, a Listeria monocytogenes antigen, or a West Nile Virus antigen.

The invention is also directed to a method of modulating TLR signaling in a subject comprising administering to a subject in need thereof one of the aforementioned vaccines or polypeptides of the invention. In preferred embodiments, the subject is a mammal.

The invention is also directed to a method of modulating TLR signaling in a cell comprising contacting a cell, wherein the cell comprises the TLR, with one of the aforementioned polypeptides of the invention. In preferred embodiments, the cell is a mammalian cell.

### DESCRIPTION OF THE DRAWINGS

FIG. 1 depicts known interactions of PAMPs with various Toll-like Receptors (TLRs). (G+=Gram-positive. (G−)=Gram-negative.

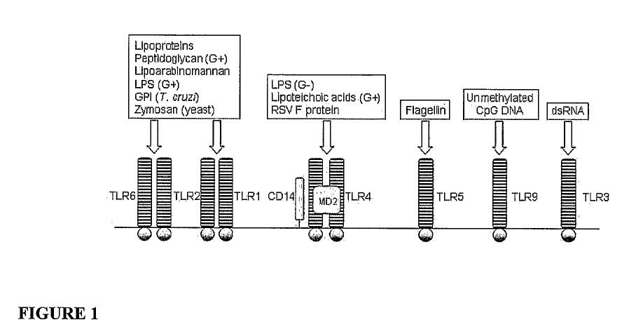

FIG. 2 is a schematic depicting the steps of the phage display screening assay (“biopanning” assay) strategy for identification of phage displaying polypeptide TLR ligands.

FIG. 3 is a bar graph showing activation of NF-κB-dependent luciferase activity in 293 (“293”) and 293.hTLR5 (“293/hTLR5”) cells exposed to T7 phage displaying the fliC protein (“Phage”, black bar) or to medium alone (“Medium”, striped bar); and in 293.hTLR5 cells exposed to T7 phage displaying the S-tag polypeptide. (“S-Tag”, “Phage”, black bar) or to medium alone (“S-Tag”, “Medium”, striped bar). “RLU”=relative luciferase units.

FIG. 4 is a bar graph depicting enrichment for TLR5-binding fliC phage using the phage display screening assay (“biopanning” assay). Results are presented as the enrichment percentage (%), calculated as the percentage of input phage recovered after each indicated round of the assay.

FIG. 5 is a bar graph depicting enrichment of TLR2-binding pentapeptide phage using the phage display screening assay (“biopanning” assay). Results are presented as the enrichment percentage (%), calculated as the percentage of input phage recovered after each indicated round of the assay.

FIG. 6 is a Coommassie stained SDS-PAGE gel of Ni-NTA purified recombinant polypeptide TLR2 ligands. Lane M=molecular weight markers. Lane 1=recombinant protein ID# 1. Lane 2=recombinant protein ID#2. Lane 3=recombinant protein ID#3.

FIG. 7 is a bar graph depicting induction of IL-8 (in pg/mL) secretion from 293 (black bar) and 293.hTLR2.hCD14 (white bar) cells exposed to Ni-NTA purified recombinant polypeptide TLR2 ligands or Pam₃Cys. Pam3=Pam₃Cys positive control. ID#1=recombinant protein ID#1. ID#2=recombinant protein ID#2. ID#3=recombinant protein ID#3. Left panel includes the PaM₃Cys control, whereas the right panel shows only the Ni-NTA purified recombinant polypeptide TLR2 ligands.

FIG. 8 depicts a schematic of exemplary plasmid vector T7.LIST. T7.LIST is designed to express recombinant LLO-p60 (SEQ ID NO: 39) protein with a V5 epitope (SEQ ID NO: 40) and a polyhistidine tag (6×His). T7=T7 promoter. rbs=ribosome binding site.

FIG. 9 depicts the amino acid sequence of human TLR2 (SEQ ID NO: 4).

### DETAILED DESCRIPTION

The present invention provides novel methods to identify polypeptide TLR ligands. The method of the invention comprises the steps of: a) providing a multiplicity of test phage in the form of a phage display library, wherein each individual test phage comprises a nucleic acid insert encoding a test polypeptide; b) contacting a TLR^{lo} cell with the multiplicity of test phage; c) retaining the test phage that do not bind to the TLR^{lo} cell; d) contacting a TLR^{hi} cell, wherein the TLR is the same TLR as in step b), with the test phage retained in step c); e) retaining the test phage that bind to the TLR^{hi} cell; f) amplifying the test phage retained in step e); g) optionally, repeating steps a) through f); and h) characterizing the polypeptide encoded by the nucleic acid insert of a test phage amplified in step f), wherein the polypeptide characterized in step h) is a polypeptide TLR ligand. In preferred embodiments, the steps a) through f) are performed at least 4 times.

In the method of the invention, step b) serves to remove those phage that bind non-specifically to the cell TLR^{lo}. Specifically, this step serves to remove those phage that bind to TLRs other than the target TLR, where TLRs other than the target TLR are expressed by the TLR^{lo} cell. Conversely, step d) serves to retain those phage that bind specifically to the TLR of interest.

Thus, upon iteration of the steps of the method, the phage population is dramatically enriched for those phage that specifically bind to the TLR of interest, while phage with other binding activities are selectively depleted from the phage population. In each round of biopanning, the harvested phage that are bound to TLR^{hi} cells can be titred prior to amplification, amplified, and then titred again prior to initiation of the next cycle of biopanning. In this way, it is possible to determine the percent (%) of input phage in each cycle that are ultimately harvested from the TLR^{hi} cells. This calculation provides a round-by-round measure of enrichment within the phage display library for phage that display TLR-binding peptides.

### Toll-Like Receptors (TLRs)

As used herein, the term “Toll-like Receptor” or “TLR” refers to any of a family of pattern recognition receptor (PRR) proteins that are homologous to the Drosophila melanogaster Toll protein. TLRs are type I transmembrane signaling receptor proteins that are characterized by an extracellular leucine-rich repeat domain and an intracellular domain homologous to that of the interleukin 1 receptor. The TLR family includes, but is not limited to, mammalian TLRs 1 through 11 and 13, including mouse and human TLRs 1-11 and 13. In preferred embodiments, the TLR is TLR2, TLR4 or TLR5.

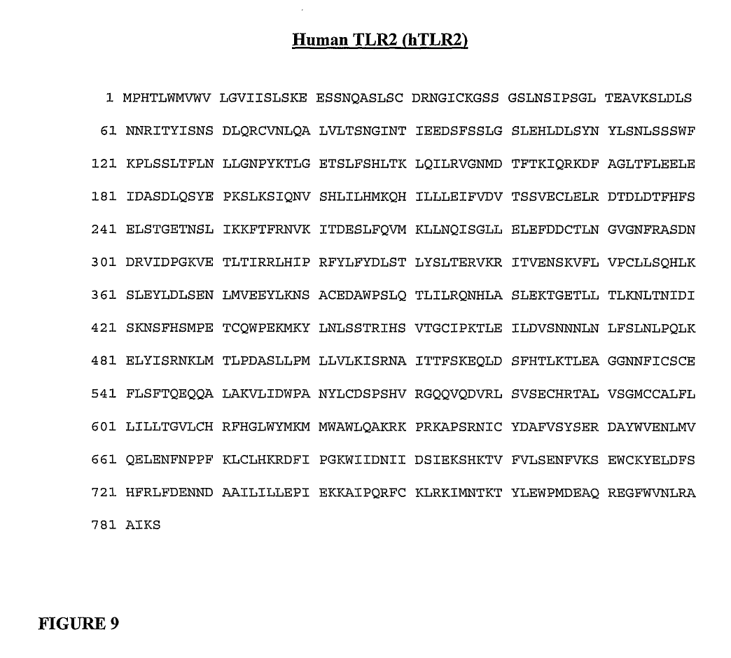

Toll-like receptor 2 (TLR2) is involved in the recognition of, e.g., multiple products of Gram-positive bacteria, mycobacteria and yeast, including LPS and lipoproteins. TLR2 is known to heterodimerize with other TLRs, a property believed to extend the range of PAMPs that TLR2 can recognize. For example, TLR2 cooperates with TLR6 in the response to peptidoglycan and diacylated mycoplasmal lipopeptide, and associates with TLR^{hi} to recognize triacylated lipopeptides. Pathogen recognition by TLR2 is strongly enhanced by CD14. The nucleotide and amino acid sequence for TLR2 has been reported for a variety of species, including, mouse, human, Rhesus monkey, rat, zebrafish, dog, pig and chicken. The nucleotide and amino acids sequences of mouse TLR2 are set forth in SEQ ID NOs: 1 and 2, respectively. The nucleotide and amino acid sequences of human TLR2 are set forth in SEQ ID NOs: 3 and 4, respectively. The amino acid sequence of human TLR2 is shown in FIG. 9 (SEQ ID NO: 4). In preferred embodiments, TLR2 is a mammalian TLR2. In particularly preferred embodiments, TLR2 is mouse TLR2 (mTLR2) or human TLR2 (hTLR2).

TLR4, the first human TLR identified, is the receptor for Gram-negative lipopolysaccharide (LPS). The TLR4 gene was shown to be mutated in C3H/HeJ and C57BL/10ScCr mice, both of which are low responders to lipopolysaccharide (LPS). TLR4 requires MD-2, a secreted molecule, to functionally interact with LPS. A third protein, called CD14, participates in LPS signaling, leading to NF-κB translocation. This signaling is mediated through the adaptor protein MyD88 but also through a MyD88-independent pathways that involves the (TIR) domain-containing adapter protein (TIRAP). The nucleotide and amino acid sequence for TLR4 has been reported for a variety of species, including, mouse, human, gorilla, rat, horse, dog, pig, rabbit and cow. The nucleotide and amino acids sequences of mouse TLR4 are set forth in SEQ ID NOs: 80 and 81, respectively. A variety of TLR4 isoforms have been identified for human TLR4. The nucleotide and amino acid sequences of human TLR4 isoform A are set forth in SEQ ID NOs: 82 and 83, respectively. The nucleotide and amino acid sequences of human TLR4 isoform C are set forth in SEQ ID NOs: 84 and 85, respectively. In preferred embodiments, TLR4 is a mammalian TLR4. In particularly preferred embodiments, TLR4 is mouse TLR4 (mTLR4) or human TLR4 (hTLR4).

TLR5 is the Toll-like receptor that recognizes flagellin from both Gram-positive and Gram-negative bacteria. Activation of the receptor stimulates the production of proinflammatory cytokines, such as TNFα, through signaling via the adaptor protein MyD88 and the serine kinase IRAK. TLR5 can generate a proinflammatory signal as a homodimer suggesting that it might be the only TLR required for flagellin recognition. The nucleotide and amino acid sequence for TLR5 has been reported for a variety of species, including, mouse, human, rat, dog, Xenopus, rainbow trout, chimpanzee, cat, cow, and zebrafish. The nucleotide and amino acids sequences of mouse TLR5 are set forth in SEQ ID NOs: 86 and 87, respectively. The nucleotide and amino acid sequences of human TLR5 are set forth in SEQ ID NOs: 88 and 89, respectively. In preferred embodiments, TLR5 is a mammalian TLR5. In particularly preferred embodiments, TLR5 is mouse TLR5 (mTLR5) or human TLR5 (hTLR5).

### Polypeptide TLR Ligands

The terms “polypeptide ligand for TLR” and “polypeptide TLR ligand” are used interchangeably herein. By the term “polypeptide TLR ligand” is meant a polypeptide that binds to the extracellular portion of a TLR protein. For example, in context of the present invention, novel polypeptide TLR ligands are identified based upon their ability to bind to the extracellular domain of a TLR protein in a phage display-based “biopanning” assay. In preferred embodiments, the polypeptide TLR ligands of the invention are functional TLR ligands, i.e. they modulate TLR signaling. As used herein, the term “TLR signaling” refers to any intracellular signaling pathway initiated by a given activated TLR, including shared pathways (e.g., activation of NF-κB) and TLR-specific pathways. As used herein the term “modulating TLR signaling” includes both activating (i.e. agonizing) TLR signaling and suppressing (i.e. antagonizing) TLR signaling. Thus, a polypeptide TLR ligand that modulates TLR signaling agonizes or antagonizes TLR signaling. Without intending to be limited by mechanism, it is believed that the polypeptide TLR ligands modulate TLR signaling by binding to the extracellular portion of the target TLR, thereby modulating the intracellular signaling cascade(s) of the target TLR.

As used herein, the term “polypeptide” or “protein” refers to a polymer of amino acid monomers that are alpha amino acids joined together through amide bonds. The terms “polypeptide” and “protein” are used interchangeably herein. Polypeptides are therefore at least two amino acid residues in length, and are usually longer. Generally, the term “peptide” refers to a polypeptide that is only a few amino acid residues in length, e.g. from three to 50 amino acid residues. A polypeptide, in contrast with a peptide, may comprise any number of amino acid residues. Hence, the term polypeptide includes peptides as well as longer sequences of amino acids.

As used herein, the term “positively charged amino acid” refers to an amino acid selected from the group consisting of lysine (Lys or K), arginine (Arg or R), and Histidine (His or H). The percent (%) positively charged amino acids of a polypeptide is calculated as (Total number of K+R+H amino acids of polypeptide)/(Total amino acid length of polypeptide).

Amino acid residues are abbreviated as follows: Phenylalanine is Phe or F; Leucine is Leu or L; Isoleucine is Ile or I; Methionine is Met or M; Valine is Val or V; Serine is Ser or S; Proline is Pro or P; Threonine is Thr or T; Alanine is Ala or A; Tyrosine is Tyr or Y; Histidine is His or H; Glutamine is Gln or Q; Asparagine is Asn or N; Lysine is Lys or K; Aspartic Acid is Asp or D; Glutamic Acid is Glu or E; Cysteine is Cys or C; Tryptophan is Trp or W; Arginine is Arg or R; and Glycine is Gly or G.

The identified polypeptide TLR ligands will find utility in a variety of applications. For example, the identified polypeptide TLR ligands may be used in methods of modulating TLR signaling. The identified polypeptide TLR ligands may also be used in novel polypeptide TLR-ligand:antigen vaccines.

### TLR^{lo} Cells and TLR^{hi} Cells

As used herein the terms “TLR^{lo}” and “TLR^{hi}” are comparative terms referring to the expression level of a given TLR in a cell to be used in the method of the invention. Thus, a TLR^{lo} cell has a relatively low level of expression of a given TLR and a TLR^{hi} cell has a relatively high level of expression of the same TLR. In one embodiment, the TLR^{lo} cell is a cell that does not endogenously express a given TLR and the TLR^{hi} cell is a cell that does endogenously express the same TLR. In another embodiment, the TLR10 cell is a cell that endogenously expresses a given TLR and the TLR^{hi} cell is a cell that endogenously expresses the same TLR to a higher degree. In another embodiment, the TLR^{lo} cell is a cell that endogenously expresses a given TLR and the TLR^{hi} cell is a cell that ectopically expresses the same TLR to a higher degree. In another embodiment, the TLR^{lo} cell is a cell that ectopically expresses a given TLR and the TLR^{hi} cell is a cell that ectopically expresses the same TLR to a higher degree. In another embodiment, the TLR^{hi} cell is a cell that endogenously expresses a given TLR and the TLR^{lo} cell is a cell in which endogenous expression of the given TLR has been abrogated (e.g., by mutation).

In preferred embodiments, the level of expression of TLRs other than the given TLR are comparable in the TLR^{lo} cell and the TLR^{hi} cell. For example, the TLR^{lo} cell may be a cell that does not endogenously express TLR2 but which does endogenously express TLR4 and TLR5, while the TLR^{hi} cell is a cell that endogenously expresses TLR2, TLR4 and TLR5. In another example, the TLR^{lo} cell is a cell of a particular TLR expression profile and the TLR^{hi} cell is generated by causing ectopic expression of the chosen TLR in the TLR^{lo} cell. In this case, the principal difference between the TLR^{lo} cell and the TLR^{hi} cell is in expression level of the chosen TLR. In another example, the TLR^{hi} cell is a cell of a particular TLR expression profile and the TLR^{lo} cell is generated by abrogating expression of the chosen TLR in the TLR^{hi} cell (e.g., by mutation). In this case, the principal difference between the TLR^{lo} cell and the TLR^{hi} cell is in expression level of the chosen TLR.

Exemplary cells to be used in the methods of the invention include various strains of E. coli, yeast, Drosophila cells (e.g. S-2 cells), and mammalian cells. In preferred embodiments the TLR^{lo} cell and the TLR^{hi} cell are the same cell type. However, the invention also contemplates methods wherein the TLR^{lo} cell and the TLR^{hi} cell are different cell types.

In preferred embodiments at least one of the cells (i.e., the TLR^{lo} cell or the TLRₕᵢ cell) is a mammalian cell. In preferred embodiments at least one of the cells (i.e., the TLR^{lo} cell or the TLR^{hi} cell) is a HEK293 cell, a RAW264.7 cell (ATCC Accession # TIB-71), or a NIH3T3 cell. In particularly preferred embodiments the TLR^{lo} cell and the TLR^{hi} cell are both mammalian cells. In particularly preferred embodiments the TLR^{lo} cell and the TLR^{hi} cell are both a HEK293 cell, both a RAW264.7 cell, or both a NIH3T3 cell.

The TLR expression profile of a cell may be determined by any of the methods well known in the art, including Western blotting, immunoprecipitation, flow cytometry/FACS, immunohistochemistry/immunocytochemistry, Northern blotting, RT-PCR, whole mount in situ hybridization, etc. For example, monoclonal and polyclonal antibodies to human or mouse TLR2 are commercially available, e.g., from Active Motif, BioVision, IMGENEX, R&D Systems, ProSci, Cellsciences, and eBioscience. For example, human TLR2 and mouse/rat TLR2 primer pairs are commercially available, e.g., from R&D Systems and Bioscience Corporation. For example, monoclonal and polyclonal antibodies to human or mouse TLR4 are commercially available, e.g., from BioVision, Cell Sciences, IMGENEX, Novus Biologicals, R&D Systems, Serotec Inc., Stressgen Bioreagents, and Zymed. For example, mouse TLR4 primer pairs are commercially available, e.g., from Bioscience Corporation. For example, monoclonal and polyclonal antibodies to human or mouse TLR5 are commercially available, e.g., from BD Biosciences, BioVision, IMGENEX, and Zymed. For example, SuperArray RT-PCR Profiling Kits for simultaneous quantitation of the expression of mouse TLRs 1 through 9 or human TLRs 1 through 10 are available from Bioscience Corporation.

Cells known to endogenously express TLR2 include dendritic cells, macrophages, natural killer cells, B-cells, epithelial cells, NIH3T3 cells, and RAW264.7 cells. Cells known not to endogenously express TLR2 include HEK293 cells. Cells known to endogenously express TLR4 include dendritic cells, macrophages, natural killer cells, B-cells, NIH3T3 cells, and RAW264.7 cells. Cells known not to endogenously express TLR4 include HEK293 cells. Cells known to endogenously express TLR5 include HEK293 cells, dendritic cells, macrophages, and epithelial cells, especially gut epithelium. Cells known not to endogenously express TLR5 include RAW264.7 cells, and 293T/17 cells (ATCC # CRL-11268).

Cells that ectopically express TLRs may be generated by standard techniques well known in the art. For example, a nucleic acid sequence encoding a TLR may be introduced into a cell. Such nucleic acids may be obtained by any of the synthetic or recombinant DNA methods well known in the art. See, for example, DNA Cloning: A Practical Approach Vol I and II (Glover ed.:1985); Oligonucleotide Synthesis (Gait ed.:1984); Transcription And Translation (Hames & Higgins, eds.:1984); Perbal. A Practical Guide To Molecular Cloning (1984); Ausubel et al., eds. Current Protocols in Molecular Biology, (John Wiley & Sons, Inc.: 1994); PCR Primer: A Laboratory Manual, 2^{nd} Edition. Dieffenbach and Dveksler, eds. (Cold Spring Harbor Laboratory Press: 2003); and Sambrook et al. Molecular Cloning: A Laboratory Manual, 3^{rd} Edition (Cold Spring Harbor Laboratory Press: 2001).

Ectopic expression of a TLR may be achieved, for example, by recombinant expression of an expression construct encoding the TLR. In such an expression construct, a nucleic acid sequence encoding the TLR is operatively associated with expression control sequence elements which provide for the proper transcription and translation of the TLR ligand within the chosen host cells. Such sequence elements may include a promoter, a polyadenylation signal, and optionally internal ribosome entry sites (IRES) and other ribosome binding site sequences, enhancers, response elements, suppressors, signal sequences, and the like. Codon selection, where the target nucleic acid sequence of the construct is engineered or chosen so as to contain codons preferentially used within the desired host call, may be used to minimize premature translation termination and thereby maximize expression.

The nucleic acid sequence may also encode a peptide tag for easy identification and purification of the translated TLR. Preferred peptide tags include GST, myc, His, and FLAG tags. The encoded peptide tag may include recognition sites for site-specific proteolysis or chemical agent cleavage to facilitate removal of the peptide tag. For example a thrombin cleavage site could be incorporated between a TLR and its peptide tag.

The promoter sequences may be endogenous or heterologous to the host cell to be modified, and may provide ubiquitous (i.e., expression occurs in the absence of an apparent external stimulus) or inducible (i.e., expression only occurs in presence of particular stimuli) expression. Promoters that may be used to control gene expression include, but are not limited to, cytomegalovirus (CMV) promoter (U.S. Pat. No. 5,385,839 and No. 5,168,062), the SV40 early promoter region (Benoist and Chambon. Nature 1981; 290:304-310), the promoter contained in the 3′ long terminal repeat of Rous sarcoma virus (Yamamoto et al. Cell 1980; 22:787-797), the herpes thymidine kinase promoter (Wagner et al. Proc. Natl. Acad. Sci. USA 1981; 78:1441-1445), the regulatory sequences of the metallothionein gene (Brinster et al. Nature 1982; 296:39-42); prokaryotic promoters such as the alkaline phosphatase promoter, the trp-lac promoter, the bacteriophage lambda P_{L} promoter, the T7 promoter, the beta-lactamase promoter (VIIIa-Komaroff et al. Proc. Natl. Acad. Sci. USA 1978; 75:3727-3731), or the tac promoter (DeBoer et al. Proc. Natl. Acad. Sci. USA 1983; 80:21-25); and promoter elements from yeast or other fungi such as the Gal4 promoter, the ADC (alcohol dehydrogenase) promoter, and the PGK (phosphoglycerol kinase) promoter.

The expression constructs may further comprise vector sequences that facilitate the cloning and propagation of the expression constructs. A large number of vectors, including plasmid and fungal vectors, have been described for replication and/or expression in a variety of eukaryotic and prokaryotic host cells. Standard vectors useful in the current invention are well known in the art and include (but are not limited to) plasmids, cosmids, phage vectors, viral vectors, and yeast artificial chromosomes. The vector sequences may contain, for example, a replication origin for propagation in E. coli; the SV40 origin of replication; an ampicillin, neomycin, or puromycin resistance gene for selection in host cells; and/or genes (e.g., dihydrofolate reductase gene) that amplify the dominant selectable marker plus the nucleic acid of interest. For example, a plasmid is a common type of vector. A plasmid is generally a self-contained molecule of double-stranded DNA, usually of bacterial origin, that can readily accept additional foreign DNA and that can readily be introduced into a suitable host cell. A plasmid vector generally has one or more unique restriction sites suitable for inserting foreign DNA. Examples of plasmids that may be used for expression in prokaryotic cells include, but are not limited to, pBR322-derived plasmids, pEMBL-derived plasmids, pEX-derived plasmids, pBTac-derived plasmids, pUC-derived plasmids, and pET-LIC-derived plasmids.

Techniques for introduction of nucleic acids to host cells are well established in the art, including, but not limited to, electroporation, microinjection, liposome-mediated transfection, calcium phosphate-mediated transfection, or virus-mediated transfection. See, for example, Felgner et al., eds. Artificial self-assembling systems for gene delivery. (Oxford University Press:1996); Lebkowski et al. Mol Cell Biol 1988; 8:3988-3996; Sambrook et al. Molecular Cloning: A Laboratory Manual. 2^{nd} Edition (Cold Spring Harbor Laboratory:1989); and Ausubel et al., eds. Current Protocols in Molecular Biology (John Wiley & Sons:1989).

Expression constructs encoding TLRs may be transfected into host cells in vitro. Exemplary host cells include various strains of E. coli, yeast, Drosophila cells (e.g. S-2 cells), and mammalian cells. Preferred in vitro host cells are mammalian cell lines.

For example, pUNO-TLR plasmids for TLRs 1 through 11 and TLR13 are available from Invivogen. These plasmids provide for high level TLR expression in mammalian host cells (e.g., HEK293 and NIH3T3 cells). Protocols for in vitro culture of mammalian cells are well established in the art. See, for example, J. Masters, ed. Animal Cell Culture: A Practical Approach 3^{rd} Edition. (Oxford University Press: 2000) and Davis, ed. Basic Cell Culture 2^{nd} Edition. (Oxford University Press:2002).

### Phage Display Libraries

As discussed above, phage display is a selection technique in which a peptide or protein is genetically fused to a coat protein of a bacteriophage. The fusion protein is displayed on the exterior of the phage virion, while the DNA encoding the fusion protein resides within the virion. This physical linkage between the displayed protein and the DNA encoding it allows for screening of vast numbers of variants of the protein by a simple in vitro selection procedure termed “biopanning”. Phage display technology offers a very powerful tool for the isolation of new ligands from large collections of potential ligands including short peptides, antibody fragments and randomly modified physiological ligands to receptors. These systems have been effectively employed in studies of structural and functional aspects of receptor-ligand interactions using either purified receptors immobilized on a polymer surface or receptors in their natural environment on the surface of living cells. The terms “bacteriophage” and “phage” are used interchangeably herein.

As used herein the term “phage display library” refers to a collection of phage wherein each individual phage of the collection comprises a polypeptide genetically fused to a coat protein of the phage such that the fusion protein is displayed on the exterior of the phage virion, while the nucleic encoding the fusion protein resides within the phage. The nucleic acid residing within the phage comprises phage DNA and at least one nucleic acid insert inserted within a portion of the phage DNA encoding a phage coat protein. The size of a phage display library refers to the total number of phage in a library. The complexity of a phage display library refers to the total number of different phage (i.e., number of different nucleic acid inserts encoding different fusion proteins) in a library. For example, a library containing a total of 10³ phage, wherein the phage all comprise the same fusion protein has a size of 10³ and a complexity of 1. Preferably, a phage display library will have high degree of complexity as well as a large size.

Techniques for the construction of phage display libraries are well known in the art. See, for example, Smith. Science 1985; 228:1315-1317; Scott and Smith. Science 1990; 249:386-390; Smith and Scott. Meth Enz 1993; 217:228-257; Smith and Petrenko. Chem. Rev 1997; 97:391-410; Hufton et al. J Immunol Methods 1999; 231:39-51, and Barbas et al., eds. Phage Display: A Laboratory Manual (CSHL Press: 2001).

Phage suitable for use in construction of phage display libraries include non-lytic phage (e.g., M13 bacterial filamentous phage) and lytic phage (e.g., lambda-, T7-, and T4-based phage). A variety of phage vectors suitable for use in construction of phage display libraries are commercially available, for example, from Novagen, New England Biolabs, and Spring Bioscience.

For example, one type of phage display library is a biased peptide library (BPL). BPLs include libraries comprised of phage displaying overlapping peptides spanning a known polypeptide of interest. BPLs based on known TLR-binding polypeptides are particularly suitable for use in the methods of the invention. Such BPLs can be used to identify the minimal peptide sequences within the known protein that are responsible for binding to the target TLR. For example, libraries of phage displaying overlapping peptides (e.g., between 5 and 20 amino acids) spanning the entire region of Measles Virus hemagglutinin (HA, a TLR2 ligand), respiratory syncytial virus fusion protein (RSV F, a TLR4 ligand), or E. coli flagellin (fliC, a TLR5 ligand) may be constructed. For example, to construct a BPL, synthetic oligonucleotides covering the entire coding region of the polypeptide of interest are converted to double-stranded molecules, digested with EcoRI and HindIII restriction enzymes, and ligated into the T7SELECT bacteriophage vector (Novagen). The ligation reactions are packaged in vitro and amplified by either the plate or liquid culture method (according to manufacturer's instructions). The amplified phages are titred (according to manufacturer's instructions) to evaluate the total number of independent clones present in the library (i.e., the complexity of the library). In preferred embodiments the complexity of a BPL is at least 10². In particularly preferred embodiments the complexity of a BPL is at least 10³.

Another type of phage display library is a random peptide library (RPL). For example, libraries of phage displaying random peptides of from 5 to 30 amino acids in length are constructed essentially as described above for biased peptide libraries, but utilizing oligonucleotides of defined length and random sequences. Such RPLs may used to identify polypeptide ligands of TLRs. In preferred embodiments the complexity of a RPL is at least 10⁷. In particularly preferred embodiments the complexity of a RPL is at least 10⁹. It is preferred that RPLs be constructed with only 32 codons (e.g. in the form NNK or NNS where N=A/T/G/C; K=G/T; S=G/C), thus reducing the redundancy inherent in the genetic code from a maximum codon number of 64 to 32 by eliminating redundant codons. Thus, for example, a 6-amino acid residue library displaying all possible hexapeptides requires 32⁶ (˜10⁹) unique clones.

Another type of phage display library is a biased, random peptide library. In such libraries a known polypeptide TLR ligand is subjected to structure-function analysis by random mutation of the various positions of the polypeptide (i.e., different amino acid positions are coordinately or independently randomized). Such a library may be used to identify the critical amino acid residues for TLR binding within a known polypeptide TLR ligand and/or to identify sequence variants of known polypeptide TLR ligands that exhibit altered TLR binding specificity and/or activity. For example, as discussed above the pentapeptide ALTTE is a known polypeptide TLR2 ligand. A biased, random peptide library may be constructed representing each of the sequences XLTTE, AXTTE, ALXTE, ALTXE and ALTTX, and/or XXTTE, AXXTE, ALXXE, ALTXX, etc (wherein X=any amino acid). Such a library may be constructed essentially as described above for biased peptide libraries, utilizing oligonucleotides of 15 nucleotides in length and the appropriate sequences.

Another type of phage display library is based on a cDNA library. For example, libraries of phage displaying bacterial-derived polypeptides may be constructed as described above for biased peptide libraries using cDNA derived from a microbial, e.g., bacterial source of choice. Such cDNA libraries may be used to identify polypeptide TLR ligands from particular pathogenic or non-pathogenic microbes. In order to obtain bacterial cDNA, bacterial mRNA is isolated and reversed-transcribed into cDNA. For example, a PCR-ready single-stranded cDNA library made from total RNA of E. coli strain C600 is commercially available (Qbiogene).

Another type of phage display library is a constrained, cyclic peptide library. In such libraries, each peptide insert (e.g. a random peptide of from 5 to 30 amino acids in length) is flanked by cysteine residues (e.g., the peptide insert is of the sequence Cys-Nₓ-Cys). These cysteine residues form a disulfide bond, forcing the peptide insert into a loop or cyclic structure. This cyclization restricts conformational freedom, stabilizing the functional presentation of the peptide insert and potentially improving binding affinity of the peptide insert for target sites due to a reduction in entropy.

A variety of pre-made phage display libraries, including random peptide libraries and human and mouse cDNA libraries, are commercially available, for example, from Novagen, New England Biolabs, and Spring Bioscience.

Methods for the amplification and isolation of phage (e.g., of phage display libraries) are well known in the art. See, for example, Barbas et al., eds. Phage Display: A Laboratory Manual (CSHL Press: 2001).

### Characterization of the Polypeptide Encoded by a Nucleic Acid Insert

The polypeptide encoded by a nucleic acid insert of a phage may be characterized by any of the methods well established in the art, including, but not limited to, nucleic acid sequencing of the nucleic acid insert, deduction of the polypeptide sequence from the nucleic acid sequence of the insert, direct determination of polypeptide sequence, and analysis of the biological activity of the encoded polypeptide.

For example, nucleic acid inserts of individual T7Select phage may amplified by PCR using the commercially available primers T7SelectUP (5′-GGA GCT GTC GTA TTC CAG TC-3′; SEQ ID NO: 37; Novagen, catalog #70005) and T7SelectDOWN (5′-AAC CCC TCA AGA CCC GTT TA-3′; SEQ ID NO: 38; Novagen, catalog #70006). The PCR product DNA may purified using the QIAquick 96 PCR Purification Kit (Qiagen) and subjected to DNA sequencing using T7SelectUP and T7SelectDOWN primers. The amino acid sequence of the encoded polypeptide may then be deduced from the nucleic acid sequence based upon the known genetic code.

In another example, the polypeptide encoded by a nucleic acid insert may be generated by coupled in vitro transcription and translation (e.g., as described in Example 6, below). Kits for in vitro transcription and translation are available from a wide variety of commercial sources including Promega, Ambion, Roche Applied Science, Novagen, Invitrogen, PanVera, and Qiagen. For example, kits for in vitro translation using reticulocyte or wheat germ lysates are commercially available from Ambion. For example, using the rabbit reticulocyte lysate system, reticulocyte lysate is programmed with the PCR DNA using a TNT T7 Quick for PCR DNA kit (Promega), which couples transcription to translation. To initiate a TNT reaction, the DNA template is incubated at 30° C. for 60-90 min in the presence of rabbit reticulocyte lysate, RNA polymerase, an amino acid mixture and RNAsin ribonuclease inhibitor.

Direct peptide sequencing may be performed, e.g., on the in vitro transcribed and translated polypeptide, to determine the amino acid sequence of the polypeptide encoded by a nucleic acid insert.

An in vitro transcribed and translated polypeptide may be further characterized, e.g., its activity to modulate TLR signaling may be confirmed. The ability of the nucleic acid insert-encoded polypeptide to modulate TLR signaling may be assessed using a variety of assay systems well known in the art.

In one embodiment, the ability of a polypeptide to modulate TLR signaling is measured in a dendritic cell (DC) activation assay. For this assay murine or human dendritic cell cultures may be obtained. For example, murine DCs may be generated in vitro as previously described (see, for example, Lutz et al. J Immun Meth. 1999; 223:77-92). In brief, bone marrow cells from 6-8 week old C57BL/6 mice are isolated and cultured for 6 days in medium supplemented with 100 U/ml GMCSF (Granulocyte Macrophage Colony Stimulating Factor), replenishing half the medium every two days. On day 6, nonadherant cells are harvested and resuspended in medium without GMSCF and used in the DC activation assay. For example, human DCs may obtained commercially (for example, from Cambrex, Walkersville, Md.) or generated in vitro from peripheral blood obtained from healthy donors as previously described (see, for example, Sallusto and Lanzavecchia. J Exp Med 1994; 179:1109-1118). In brief, peripheral blood mononuclear cells (PBMC) are isolated by Ficoll gradient centrifugation. Cells from the 42.5-50% interface are harvested and further purified following magnetic bead depletion of B- and T-cells using antibodies to CD19 and CD2, respectively. The resulting DC enriched suspension is cultured for 6 days in medium supplemented with 100 U/ml GMCSF and 1000 U/ml IL-4 (Interleukin-4). On day 6, nonadherant cells are harvested and resuspended in medium without cytokines and used in the DC activation assay. For example, in a dendritic cell assay, a polypeptide TLR ligand may be added to DC cells in culture and the cultures incubated for 16 hours. Supernatants may be harvested, and cytokine (e.g., IFNγ, TNFα, IL-12, IL-10 and/or IL-6) concentrations may be determined, e.g., by sandwich enzyme-linked immunosorbent assay (ELISA) using matched antibody pairs (commercialy available, for example, from BD Pharmingen or R&D Systems) following the manufacturer's instructions. Cells may be harvested, and co-stimulatory molecule expression (e.g., B7-2) determined by flow cytometry using antibodies (commercially available, for example, from BD Pharmingen or Southern Biotechnology Associates) following the manufacturer's instructions. Analysis may be performed on a Becton Dickinson FACScan running Cellquest software. Functional polypeptide TLR ligands modulate cytokine and/or co-stimulatory molecule expression in the DC assay.

In another embodiment, the ability of a polypeptide to modulate expression of an NF-κB-reporter gene in a TLR-dependent manner is assessed. As discussed above, one of the shared pathways of TLR signaling results in the activation of the transcription factor NF-κB. Therefore, expression of an NF-κB-dependent reporter gene can serve as an indicator of TLR signaling. In such an assay, the ability of a polypeptide TLR ligand to modulate expression of an NF-κB-dependent reporter gene in a TLR^{lo} cell versus in a TLR^{hi} cell may be compared. For example, a polypeptide TLR ligand may induce NF-κB-dependent reporter gene expression to a greater extent in a TLR^{hi} cell than in a TLR^{lo} cell. For example, HEK293 do not express detectable levels of endogenous TLR2. HEK293 cells harboring an NF-κB-dependent luciferase reporter gene, and ectopically expressing human or mouse TLR2 are available from Invivogen (Catalogue numbers 293-htlr2 and 293-mtlr2, respectively). For example, in such an assays, HEK293-TLR2 cells may grown in standard Dulbecco's Modified Eagle Medium (DMEM) medium with 10% Fetal Bovine Serum (FBS) supplemented with blasticidin (10 μg/ml) and then exposed to peptide ligands. Luciferase activity may be quantitated using commercial reagents.

In another embodiment, the ability of a polypeptide to modulate interleukin-8 (IL-8) expression in a TLR-dependent manner is assessed. In such an assay, the ability of a polypeptide TLR ligand to modulate IL-8 expression in a TLR^{lo} cell versus in a TLR^{hi} cell may be compared. For example, a polypeptide TLR ligand may induce IL-8 expression to a significantly greater extent expression in a TLR hi cell than in a TLR^{lo} cell. For example, HEK293 do not express detectable levels of endogenous TLR2. HEK293 cells ectopically expressing human or mouse TLR2 are available from Invivogen (Catalogue numbers 293-htlr2 and 293-mtlr2, respectively). For example, for such an assay, HEK293-TLR2 cells may be grown in standard Dulbecco's Modified Eagle Medium (DMEM) medium with 10% Fetal Bovine Serum (FBS) supplemented with blasticidin (10 μg/ml), and then exposed to a polypeptide TLR2 ligand. IL-8 expression may then be quantitated by standard methods well known in the art, including Northern Blotting to detect IL-8 mRNA, immunostaining of a Western Blot to detect IL-8 protein, and fluorescence activated cell sorter (FACS) analysis using an anti-IL-8 antibody.

### Novel Polypeptide Ligands for TLRs

The invention also relates to polypeptide ligands for TLRs, which are identified using the methods of the invention. In preferred embodiments, these novel polypeptide ligands modulate TLR signaling and thereby regulate the Innate Immune Response.

The polypeptide TLR ligands of the invention may be prepared by any of the techniques well known in the art, including translation from coding sequences and in vitro chemical synthesis.

### Translation from Coding Sequences

In one embodiment, the polypeptide TLR ligands of the invention may be prepared by translation of a nucleic acid sequence encoding the polypeptide TLR ligand. Such nucleic acids may be obtained by any of the synthetic or recombinant DNA methods well known in the art. See, for example, DNA Cloning: A Practical Approach, Vol I and II (Glover ed.: 1985); Oligonucleotide Synthesis (Gait ed.:1984); Transcription And Translation (Hames & Higgins, eds.:1984); Perbal. A Practical Guide To Molecular Cloning (1984); Ausubel et al., eds. Current Protocols in Molecular Biology, (John Wiley & Sons, Inc.:1994); PCR Primer: A Laboratory Manual, 2^{nd} Edition. Dieffenbach and Dveksler, eds. (Cold Spring Harbor Laboratory Press: 2003); and Sambrook et al. Molecular Cloning: A Laboratory Manual, 3^{rd} Edition (Cold Spring Harbor Laboratory Press: 2001). For example, nucleic acids encoding a polypeptide TLR ligand (e.g., synthetic oligo and polynucleotides) can easily be synthesized by chemical techniques, for example, the phosphotriester method (see, for example, Matteucci et al. J. Am. Chem. Soc. 1981; 103:3185-3191) or using automated synthesis methods.

Translation of the polypeptide TLR ligands of the invention may be achieved in vieo (e.g. via in vitro translation of a linear nucleic acid encoding the polypeptide TLR ligand) or in vivo (e.g. by recombinant expression of an expression construct encoding the polypeptide TLR ligand). Techniques for in vitro and in vivo expression of peptides from a coding sequence are well known in the art. See, for example, DNA Cloning: A Practical Approach, Vol I and II (Glover ed.: 1985); Oligonucleotide Synthesis (Gait ed.: 1984); Transcription And Translation (Hames & Higgins, eds.:1984); Animal Cell Culture (Freshney, ed.:1986); Perbal, A Practical Guide To Molecular Cloning (1984); Ausubel et al., eds. Current Protocols in Molecular Biology, (John Wiley & Sons, Inc.:1994); and Sambrook et al. Molecular Cloning: A Laboratory Manual, 3rd Edition (Cold Spring Harbor Laboratory Press: 2001).

In one embodiment, the polypeptide TLR ligands of the invention are prepared by in vitro translation of a nucleic acid encoding the polypeptide TLR ligand. A number of cell-free translation systems have been developed for the translation of isolated mRNA, including rabbit reticulocyte lysate, wheat germ extract, and E. coli S30 extract systems (Jackson and Hunt. Meth Enz 1983; 96:50-74; Ambion Technical Bulletin #187; and Hurst. Promega Notes 1996; 58:8). Kits for in vitro transcription and translation are available from a wide variety of commercial sources including Promega, Ambion, Roche Applied Science, Novagen, Invitrogen, PanVera, and Qiagen. For example, kits for in vitro translation using reticulocyte or wheat germ lysates are commercially available from Ambion. For example, using the rabbit reticulocyte lysate system, reticulocyte lysate is programmed with PCR DNA using a TNT T7 Quick for PCR DNA kit (Promega), which couples transcription to translation. To initiate a TNT reaction, the DNA template is incubated at 30° C. for 60-90 min in the presence of rabbit reticulocyte lysate, RNA polymerase, an amino acid mixture and RNAsin ribonuclease inhibitor.

In another embodiment, the polypeptide TLR ligands are translated from an expression construct. For a discussion of expression constructs and expression in host cells, see section TLR^{lo} cells and TLR^{hi} cells, above.

### In Vitro Chemical Synthesis

The polypeptide TLR ligands of the invention may be prepared via in vitro chemical synthesis by classical methods known in the art. These standard methods include exclusive solid phase synthesis, partial solid phase synthesis, fragment condensation, and classical solution synthesis methods (see, e.g., Merrifield. J. Am. Chem. Soc. 1963; 85:2149).

A preferred method for polypeptide synthesis is solid phase synthesis. Solid phase polypeptide synthesis procedures are well-known in the art. See, e.g., Stewart. Solid Phase Peptide Syntheses (Freeman and Co.: San Francisco: 1969); 2002/2003 General Catalog from Novabiochem Corp, San Diego, USA; and Goodman Synthesis of Peptides and Peptidomimetics (Houben-Weyl, Stuttgart:2002). In solid phase synthesis, synthesis is typically commenced from the C-terminal end of the polypeptide using an α-amino protected resin. A suitable starting material can be prepared, for example, by attaching the required α-amino acid to a chloromethylated resin, a hydroxymethyl resin, a polystyrene resin, a benzhydrylamine resin, or the like. One such chloromethylated resin is sold under the trade name BIO-BEADS SX-1 by Bio Rad Laboratories (Richmond, Calif.). The preparation of hydroxymethyl resin has been described (see, for example, Bodonszky et al. Chem. Ind. London 1966; 38:1597). Benzhydrylamine (BHA) resin has been described (see, for example, Pietta and Marshall. Chem. Commun. 1970; 650), and a hydrochloride form is commercially available from Beckman Instruments, Inc. (Palo Alto, Calif.). For example, an α-amino protected amino acid may be coupled to a chloromethylated resin with the aid of a cesium bicarbonate catalyst (see, for example, Gisin. Helv. Chim. Acta 1973; 56:1467).

After initial coupling, the α-amino protecting group is removed, for example, using trifluoroacetic acid (TFA) or hydrochloric acid (HCl) solutions in organic solvents at room temperature. Thereafter, α-amino protected amino acids are successively coupled to a growing support-bound polypeptide chain. The α-amino protecting groups are those known to be useful in the art of stepwise synthesis of polypeptides, including: acyl-type protecting groups (e.g., formyl, trifluoroacetyl, acetyl), aromatic urethane-type protecting groups [e.g., benzyloxycarboyl (Cbz) and substituted Cbz], aliphatic urethane protecting groups [e.g., t-butyloxycarbonyl (Boc), isopropyloxycarbonyl, cyclohexyloxycarbonyl], and alkyl type protecting groups (e.g., benzyl, triphenylmethyl), fluorenylmethyl oxycarbonyl (Fmoc), allyloxycarbonyl (Alloc), and 1-(4,4-dimethyl-2,6-dioxocyclohex-1-ylidene)ethyl (Dde).

The side chain protecting groups (typically ethers, esters, trityl, PMC, and the like) remain intact during coupling and are not split off during the deprotection of the amino-terminus protecting group or during coupling. The side chain protecting group must be removable upon the completion of the synthesis of the final polypeptide and under reaction conditions that will not alter the target polypeptide. The side chain protecting groups for Tyr include tetrahydropyranyl, tert-butyl, trityl, benzyl, Cbz, Z-Br—Cbz, and 2,5-dichlorobenzyl. The side chain protecting groups for Asp include benzyl, 2,6-dichlorobenzyl, methyl, ethyl, and cyclohexyl. The side chain protecting groups for Thr and Ser include acetyl, benzoyl, trityl, tetrahydropyranyl, benzyl, 2,6-dichlorobenzyl, and Cbz. The side chain protecting groups for Arg include nitro, Tosyl (Tos), Cbz, adamantyloxycarbonyl mesitoylsulfonyl (Mts), 2,2,4,6,7-pentamethyldihydrobenzofurane-5-sulfonyl (Pbf), 4-methoxy-2,3,6-trimethyl-benzenesulfonyl (Mtr), or Boc. The side chain protecting groups for Lys include Cbz, 2-chlorobenzyloxycarbonyl (2-Cl-Cbz), 2-bromobenzyloxycarbonyl (2-Br-Cbz), Tos, or Boc.

After removal of the α-amino protecting group, the remaining protected amino acids are coupled stepwise in the desired order. Each protected amino acid is generally reacted in about a 3-fold excess using an appropriate carboxyl group activator such as 2-(1H-benzotriazol-1-yl)-1,1,3,3 tetramethyluronium hexafluorophosphate (HBTU) or dicyclohexylcarbodimide (DCC) in solution, for example, in methylene chloride (CH₂Cl₂), N-methylpyrrolidone, dimethyl formamide (DMF), or mixtures thereof.

After the desired amino acid sequence has been completed, the desired polypeptide is decoupled from the resin support by treatment with a reagent, such as trifluoroacetic acid (TFA) or hydrogen fluoride (HF), which not only cleaves the polypeptide from the resin, but also cleaves all remaining side chain protecting groups. When a chloromethylated resin is used, hydrogen fluoride treatment results in the formation of the free peptide acids. When a benzhydrylamine resin is used, hydrogen fluoride treatment results directly in the free peptide amide. Alternatively, when a chloromethylated resin is employed, the side chain protected polypeptide can be decoupled by treatment of the polypeptide resin with ammonia to give the desired side chain protected amide or with an alkylamine to give a side chain protected alkylamide or dialkylamide. Side chain protection is then removed in the usual fashion by treatment with hydrogen fluoride to give the free amides, alkylamides, or dialkylamides. In preparing esters, the resins used to prepare the peptide acids are employed, and the side chain protected polypeptide is cleaved with base and the appropriate alcohol (e.g., methanol). Side chain protecting groups are then removed in the usual fashion by treatment with hydrogen fluoride to obtain the desired ester.

These procedures can also be used to synthesize polypeptides in which amino acids other than the 20 naturally occurring, genetically encoded amino acids are substituted at one, two, or more positions of any of the compounds of the invention. Synthetic amino acids that can be substituted into the polypeptides of the present invention include, but are not limited to, N-methyl, L-hydroxypropyl, L-3,4-dihydroxyphenylalanyl, δ amino acids such as L-6-hydroxylysyl and D-6-methylalanyl, L-α-methylalanyl, β amino acids, and isoquinolyl. D-amino acids and non-naturally occurring synthetic amino acids can also be incorporated into the polypeptides of the present invention.

### Polypeptide Modifications

One can also modify the amino and/or carboxy termini of the polypeptide TLR ligands of the invention. Amino terminus modifications include methylation (e.g., —NHCH₃ or —N(CH₃)₂), acetylation (e.g., with acetic acid or a halogenated derivative thereof such as α-chloroacetic acid, α-bromoacetic acid, or αc-iodoacetic acid), adding a benzyloxycarbonyl (Cbz) group, or blocking the amino terminus with any blocking group containing a carboxylate functionality defined by RCOO— or sulfonyl functionality defined by R—SO₂—, where R is selected from alkyl, aryl, heteroaryl, alkyl aryl, and the like, and similar groups. One can also incorporate a desamino acid at the N-terminus (so that there is no N-terminal amino group) to decrease susceptibility to proteases or to restrict the conformation of the polypeptide compound. For example, the N-terminus may be acetylated to yield N-acetylglycine.

Carboxy terminus modifications include replacing the free acid with a carboxamide group or forming a cyclic lactam at the carboxy terminus to introduce structural constraints. One can also cyclize the polypeptides of the invention, or incorporate a desamino or descarboxy residue at the termini of the polypeptide, so that there is no terminal amino or carboxyl group, to decrease susceptibility to proteases or to restrict the conformation of the polypeptide. C-terminal functional groups of the compounds of the present invention include amide, amide lower alkyl, amide di(lower alkyl), lower alkoxy, hydroxy, and carboxy, and the lower ester derivatives thereof, and the pharmaceutically acceptable salts thereof.

One can replace the naturally occurring side chains of the 20 genetically encoded amino acids (or the stereoisomeric D amino acids) with other side chains, for instance with groups such as alkyl, lower alkyl, cyclic 4-, 5-, 6-, to 7-membered alkyl, amide, amide lower alkyl, amide di(lower alkyl), lower alkoxy, hydroxy, carboxy and the lower ester derivatives thereof, or with 4-, 5-, 6-, to 7-membered heterocyclic. In particular, proline analogues in which the ring size of the proline residue is changed from 5 members to 4, 6, or 7 members can be employed. Cyclic groups can be saturated or unsaturated, and if unsaturated, can be aromatic or non-aromatic. Heterocyclic groups preferably contain one or more nitrogen, oxygen, and/or sulfur heteroatoms. Examples of such groups include furazanyl, furyl, imidazolidinyl, imidazolyl, imidazolinyl, isothiazolyl, isoxazolyl, morpholinyl (e.g. morpholino), oxazolyl, piperazinyl (e.g., 1-piperazinyl), piperidyl (e.g., 1-piperidyl, piperidino), pyranyl, pyrazinyl, pyrazolidinyl, pyrazolinyl, pyrazolyl, pyridazinyl, pyridyl, pyrimidinyl, pyrrolidinyl (e.g., 1-pyrrolidinyl), pyrrolinyl, pyrrolyl, thiadiazolyl, thiazolyl, thienyl, thiomorpholinyl (e.g., thiomorpholino), and triazolyl. Heterocyclic groups can be substituted or unsubstituted. Where a group is substituted, the substituent can be alkyl, alkoxy, halogen, oxygen, or substituted or unsubstituted phenyl.

One can also readily modify polypeptides by phosphorylation, and other methods (e.g., as described in Hruby et al. Biochem J. 1990; 268:249-262).

The invention also contemplates partially or wholly non-peptidic analogs of the polypeptide TLR ligands of the invention. For example, the peptide compounds of the invention serve as structural models for non-peptidic compounds with similar biological activity. Those of skill in the art recognize that a variety of techniques are available for constructing compounds with the same or similar desired biological activity as the lead peptide compound, but with more favorable activity than the lead with respect to solubility, stability, and susceptibility to hydrolysis and proteolysis (see, e.g., Morgan and Gainor. Ann. Rep. Med. Chem. 1989; 24:243-252). These techniques include replacing the polypeptide backbone with a backbone composed of phosphonates, amidates, carbamates, sulfonamides, secondary amines, or N-methylamino acids.

In one embodiment, the contemplated analogs of polypeptide TLR ligands are polypeptide-containing molecules that mimic elements of protein secondary structure (see, for example, Johnson et al “Peptide Turn Mimetics,” in Biotechnology and Pharmacy. Pezzuto et al., eds. Chapman and Hall: 1993). Such molecules are expected to permit molecular interactions similar to the natural molecule. In another embodiment, analogs of polypeptides are commonly used in the pharmaceutical industry as non-polypeptide drugs with properties analogous to those of a subject polypeptide (see, for example, Fauchere Adv. Drug Res. 1986; 15:29-69; Veber et al. Trends Neurosci. 1985; 8:392-396; and Evans et al. J. Med. Chem. 1987; 30:1229-1239), and are usually developed with the aid of computerized molecular modeling. Generally, analogs of polypeptides are structurally similar to the reference polypeptide, but have one or more peptide linkages optionally replaced by a linkage selected from the group consisting of: —CH₂NH—, —CH₂S—, —CH₂—CH₂—, —CH═CH— (cis or trans), —COCH₂—, —CH(OH)CH₂—, —CH₂SO—, and the like. See, for example, Morley Trends Pharimacol. Sci. 1980; 1:463468; Hudson et al. Int J Pept Protein Res. 1979; 14:177-185; Spatola et al. Life Sci. 1986; 38:1243-1249; Hann. J. Chem. Soc. Perkin Trans. 1982; 1:307-314; Ahnquist et al. J. Med. Chem. 1980; 23:1392-1398; Jennings-White et al. Tetrahedron Lett. 1982; 23:2533; Holladay et al. Tetrahedron Lett. 1983; 24:4401-4404; and Hruby Life Sci. 1982; 31:189-199.

Fully synthetic analogs of the polypeptide TLR ligands of the invention can be constructed by structure-based drug design through replacement of amino acids by organic moieties. See, for example, Hughes Philos. Trans. R. Soc. Lond. 1980; 290:387-394; Hodgson Biotechnol. 1991; 9:19-21; and Suckling. Sci. Prog. 1991; 75:323-359.

### Vaccines Comprising the Polypeptide TLR Ligands of the Invention

The invention also provides vaccines comprising at least one polypeptide TLR ligand identified by the method of the invention and at least one antigen. These vaccines combine both signals required for the induction of a potent adaptive immune response: an innate immune system signal (i.e. TLR signaling), and an antigen receptor signal (antigen). These vaccines may be used in methods to generate a potent antigen-specific immune response. In particular, these vaccines may used in situations where signaling through a particular TLR receptor is specifically desired.

It is particularly preferred that in the vaccines of the invention, the at least one polypeptide TLR ligand and at least one antigen are covalently linked. As used herein, the term “polypeptide TLR ligand:antigen” refers to a vaccine composition comprising at least one polypeptide TLR ligand and at least one antigen, wherein the polypeptide TLR ligand and the antigen are covalently linked. Without intending to be limited by mechanism, it is thought that covalent linkage ensures that every cell that is exposed to antigen also receives an TLR receptor innate immune signal and vice versa. However, vaccines comprising at least one polypeptide TLR ligand and at least one antigen, in which the polypeptide TLR ligand and the antigen are mixed or associated in a non-covalent fashion, e.g. electrostatic interaction, are also contemplated.

### Composition of the Vaccines of the Invention

The novel vaccines of the invention comprise at least one TLR ligand identified by the method of the invention and at least one antigen.

The antigens used in the vaccines of the present invention can be any type of antigen, including but not limited to pathogen-related antigens, tumor-related antigens, allergy-related antigens, neural defect-related antigens, cardiovascular disease antigens, rheumatoid arthritis-related antigens, other disease-related antigens, hormones, pregnancy-related antigens, embryonic antigens and/or fetal antigens and the like. The antigen component of the vaccine can be derived from sources that include, but are not limited to, bacteria, viruses, fungi, yeast, protozoa, metazoa, tumors, malignant cells, plants, animals, humans, allergens, hormones and amyloid-β peptide. The antigens may be composed of, e.g., polypeptides, lipoproteins, glycoproteins, mucoproteins, lipids, saccharides, lipopolysaccharides, nucleic acids, and the like.

Specific examples of pathogen-related antigens include, but are not limited to, antigens selected from the group consisting of West Nile Virus (WNV, e.g., envelope protein domain EIII antigen) or other Flaviviridae antigens, Listeria monocytogenes (e.g., LLO or p60 antigens), Influenza A virus (e.g., the M2e antigen), vaccinia virus, avipox virus, turkey influenza virus, bovine leukemia virus, feline leukemia virus, chicken pneumovirosis virus, canine parvovirus, equine influenza, Feline rhinotracheitis virus (FHV), Newcastle Disease Virus (NDV), infectious bronchitis virus; Dengue virus, measles virus, Rubella virus, pseudorabies, Epstein-Barr Virus, Human Immunodeficieny Virus (HIV), Simian Immunodeficiency virus (SIV), Equine Herpes Virus (EHV), Bovine Herpes Virus (BHV), cytomegalovirus (CMV), Hantaan, C. tetani, mumps, Morbillivirus, Herpes Simplex Virus type 1, Herpes Simplex Virus type 2, Human cytomegalovirus, Hepatitis A Virus, Hepatitis B Virus, Hepatitis C Virus, Hepatitis E Virus, Respiratory Syncytial Virus, Human Papilloma Virus, Salmonella, Neisseria, Borrelia, Chlamydia, Bordetella, Plasmodium, Toxoplasma, Cryptococcus, Streptococcus, Staphylococcus, Haemophilus, Diptheria, Pertussis, Escherichia, Candida, Aspergillus, Entamoeba, Giardia, and Trypanasonia.

The methods and compositions of the present invention can also be used to produce vaccines directed against tumor-associated antigens such as melanoma-associated antigens, mammary cancer-associated antigens, colorectal cancer-associated antigens, prostate cancer-associated antigens and the like. Specific examples of tumor-related or tissue-specific antigens useful in such vaccines include, but are not limited to, antigens selected from the group consisting of prostate-specific antigen (PSA), prostate-specific membrane antigen (PSMA), Her-2, epidermal growth factor receptor, gp120, and p24. In order for tumors to give rise to proliferating and malignant cells, they must become vascularized. Strategies that prevent tumor vascularization have the potential for being therapeutic. The methods and compositions of the present invention can also be used to produce vaccines directed against tumor vascularization. Examples of target antigens for such vaccines are vascular endothelial growth factors, vascular endothelial growth factor receptors, fibroblast growth factors, fibroblast growth factor receptors, and the like.

Specific examples of allergy-related antigens useful in the methods and compositions of the present invention include, but are not limited to: allergens derived from pollen, such as those derived from trees such as Japanese cedar (Cryptomeria, Cryptomeria japonica), grasses (Gramineae), such as orchard-grass (e.g. Dactylis glomerata), weeds such as ragweed (e.g. Ambrosia artemisuifolia); specific examples of pollen allergens including the Japanese cedar pollen allergens Cry j I and Cry j 2, and the ragweed allergens Amb a 1.1, Amb a 1.2, Amb a 1.3, Amnb a 1.4, Amb a II etc.; allergens derived from fungi (e.g. Aspergillus, Candida, Alternaria, etc.); allergens derived from mites (e.g. allergens from Dermatophagoides pteronyssinus, Dermatophagoides farinae etc.); specific examples of mite allergens including Der p I, Der p II, Der p III, Der p VII, Der f I, Der f II, Der f III, Der f VII etc.; house dust; allergens derived from animal skin debris, feces and hair (for example, the feline allergen Fel d I); allergens derived from insects (such as scaly hair or scale of moths, butterflies, Chironomidae etc., poisons of the Vespidae, such as Vespa mandarinia); food allergens (eggs, milk, meat, seafood, beans, cereals, fruits, nuts, vegetables, etc.); allergens derived from parasites (such as roundworm and nematodes, for example, Anisakis); and protein or peptide based drugs (such as insulin). Many of these allergens are commercially available.

Also contemplated in this invention are vaccines directed against antigens that are associated with diseases other than cancer, allergy and asthma. As one example of many, and not by limitation, an extracellular accumulation of a protein cleavage product of β-amyloid precursor protein, called “amyloid-β peptide”, is associated with the pathogenesis of Alzheimer's disease (Janus et al. Nature 2000; 408:979-982 and Morgan et al. Nature 2000; 408:982-985). Thus, the vaccines of the present invention can comprise an amyloid-β polypeptide.

The vaccines of the invention may additionally comprise carrier molecules such as polypeptides (e.g., keyhole limpet hemocyanin (KLH)), liposomes, insoluble salts of aluminum (e.g. aluminum phosphate or aluminum hydroxide), polynucleotides, polyelectrolytes, and water soluble carriers (e.g. muramyl dipeptides). A polypeptide TLR ligand and/or antigen can, for example, be covalently linked to a carrier molecule using standard methods. See, for example, Hancock et al. “Synthesis of Peptides for Use as Immunogens,” Methods in Molecular Biology: Immunochemical Protocols. Manson, ed. (Humana Press: 1992).

### Chemical Conjugates

In one embodiment, the vaccines of the invention comprise a polypeptide TLR ligand identified by the method of the invention chemically conjugated to at least one antigen. Methods for the chemical conjugation of polypeptides, carbohydrates, and/or lipids are well known in the art. See, for example, Hermanson. Bioconjugate Techniques (Academic Press; 1992); Aslam and Dent, eds. Bioconjugation: Protein coupling Techniques for the Biomedical Sciences (MacMillan: 1998); and Wong Chemistry of Protein Conjugation and Cross-linking (CRC Press: 1991). For example, in the case of carbohydrate or lipid antigens, functional amino and sulfhydryl groups may be incorporated therein by conventional chemistry. For instance, primary amino groups may be incorporated by reaction with ethylenediamine in the presence of sodium cyanoborohydride and sulfhydryls may be introduced by reaction of cysteamin dihydrochloride followed by reduction with a standard disulfide reducing agent.

Heterobifunctional crosslinkers, such as sulfosuccinimidyl (4-iodoacetyl)aminobenzoate, which link the epsilon amino group on the D-lysine residues of copolymers of D-lysine and D-glutamate to a sulfhydryl side chain from an amino terminal cysteine residue on the peptide to be coupled, may be used to increase the ratio of polypeptide TLR ligand to antigen in the conjugate.

Polypeptide TLR ligands and polypeptide antigens will contain amino acid side chains such as amino, carbonyl, hydroxyl, or sulfhydryl groups or aromatic rings that can serve as sites for linking the polypeptide TLR ligands and polypeptide antigens to each other, or for linking the polypeptide TLR ligands to an non-polypeptide antigen. Residues that have such functional groups may be added to either the polypeptide TLR ligands or polypeptide antigens. Such residues may be incorporated by solid phase synthesis techniques or recombinant techniques, both of which are well known in the art.

Polypeptide TLR ligands and polypeptide antigens may be chemically conjugated using conventional crosslinking agents such as carbodiimides. Examples of carbodiimides are 1-cyclohexyl-3-(2-morpholinyl-(4-ethyl)carbodiimide (CMC), 1-ethyl-3-(3-dimethyaminopropyl)carbodiimide (EDC), and 1-ethyl-3-(4-azonia-44-dimethylpentyl)carbodiimide.

Examples of other suitable crosslinking agents are cyanogen bromide, glutaraldehyde and succinic anhydride. In general, any of a number of homobifunctional agents including a homobifunctional aldehyde, a homobifunctional epoxide, a homobifunctional imidoester, a homobifunctional N-hydroxysuccinimide ester, a homobifunctional maleimide, a homobifunctional alkyl halide, a homobifunctional pyridyl disulfide, a homobifunctional aryl halide, a homobifunctional hydrazide, a homobifunctional diazonium derivative or a homobifunctional photoreactive compound may be used. Also included are heterobifunctional compounds, for example, compounds having an amine-reactive and a sulfhydryl-reactive group, compounds with an amine-reactive and a photoreactive group, and compounds with a carbonyl-reactive and a sulfhydryl-reactive group.

Specific examples of homobifunctional crosslinking agents include the bifunctional N-hydroxysuccinimide esters dithiobis (succinimidylpropionate), disuccinimidyl suberate, and disuccinimidyl tartarate; the bifunctional imidoesters dimethyl adipimidate, dimethyl pimelimidate, and dimethyl suberimidate; the bifunctional sulfhydryl-reactive crosslinkers 1,4-di-[3′-(2′-pyridyldithio)propion-amido]butane, bismaleimidohexane, and bis-N-maleimido-1,8-octane; the bifunctional aryl halides 1,5-difluoro-2,4-dinitrobenzene and 4,4′-difluoro-3,3′-dinitrophenylsulfone; bifunctional photoreactive agents such as bis-[b-(4-azidosalicylamide)ethyl]disulfide; the bifunctional aldehydes formaldehyde, malondialdehyde, succinaldehyde, glutaraldehyde, and adiphaldehyde; a bifunctional epoxied such as 1,4-butaneodiol diglycidyl ether; the bifunctional hydrazides adipic acid dihydrazide, carbohydrazide, and succinic acid dihydrazide; the bifunctional diazoniums o-tolidine, diazotized and bis-diazotized benzidine; the bifunctional alkylhalides N1N′-ethylene-bis(iodoacetamide), N1N′-hexamethylene-bis(iodoacetamide), N1N′-undecamethylene-bis(iodoacetamide), as well as benzylhalides and halomustards, such as a1a′-diiodo-p-xylene sulfonic acid and tri(2-chloroethyl)amine, respectively.

Examples of other common heterobifunctional crosslinking agents that may be used include, but are not limited to, SMCC (succinimidyl-4-(N-maleimidomethyl)cyclohexane-1-carboxylate), MBS (m-maleimidobenzoyl-N-hydroxysuccinimide ester), SIAB (N-succinimidyl(4-iodacteyl)aminobenzoate), SMPB (succinimidyl-4-(p-maleimidophenyl)butyrate), GMBS (N-(\ddot{γ}-maleimidobutyryloxy)succinimide ester), MPHB (4-(4-N-maleimidopohenyl)butyric acid hydrazide), M2C2H (4-(N-maleimidomethyl)cyclohexane-1-carboxyl-hydrazide), SMPT (succinimidyloxycarbonyl-á-methyl-á-(2-pyridyldithio)toluene), and SPDP (N-succinimidyl 3-(2-pyridyldithio)propionate). Crosslinking may be accomplished by coupling a carbonyl group to an amine group or to a hydrazide group by reductive amination.

In one embodiment, at least one polypeptide TLR ligand and at least one antigen are linked through polymers, such as PEG, poly-D-lysine, polyvinyl alcohol, polyvinylpyrollidone, immunoglobulins, and copolymers of D-lysine and D-glutamic acid. Conjugation of a polypeptide TLR ligand and an antigen to a polymer linker may be achieved in any number of ways, typically involving one or more crosslinking agents and functional groups on the polypeptide TLR ligand and the antigen. The polymer may be derivatized to contain functional groups if it does not already possess appropriate functional groups.

### Fusion Proteins

In preferred embodiments, the vaccines of the invention comprise a fusion protein, wherein the fusion protein comprises at least one polypeptide TLR ligand identified by the method of the invention and at least one polypeptide antigen. In one embodiment the polypeptide TLR ligand:antigen fusion protein is obtained by in vitro synthesis of the fusion protein. Such in vitro synthesis may be performed according to any methods well known in the art (see the section Novel polypeptide ligands for TLRs: In vitro chemical syntiesis, above).

In particularly preferred embodiments, the polypeptide TLR ligand:antigen fusion protein is obtained by translation of a nucleic acid sequence encoding the fusion protein. A nucleic acid sequence encoding a polypeptide TLR ligand:antigen fusion protein may be obtained by any of the synthetic or recombinant DNA methods well known in the art. See, for example, DNA Cloning: A Practical Approach, Vol I and II (Glover ed.: 1985); Oligonucleotide Synthesis (Gait ed.:1984); Transcription And Translation (Hames & Higgins, eds.:1984); Perbal, A Practical Guide To Molecular Cloning (1984); Ausubel et al., eds. Current Protocols in Molecular Biology, (John Wiley & Sons, Inc.: 1994); PCR Primer: A Laboratory Manual, 2^{nd} Edition. Dieffenbach and Dveksler, eds. (Cold Spring Harbor Laboratory Press: 2003); and Sambrook et al. Molecular Cloning: A Laboratory Manual, 3^{rd} Edition (Cold Spring Harbor Laboratory Press: 2001).

Translation of a nucleic acid sequence encoding a polypeptide TLR ligand:antigen fusion protein may be achieved by any of the in vitro or in vivo methods well known in the art (see the Section Novel polypeptide ligands for TLRs: Translation from coding sequences, above).

### Vaccine Formulations

Methods of formulating pharmaceutical compositions and vaccines are well-known to those of ordinary skill in the art (see, e.g., Remington's Pharmaceutical Sciences, 18^{th} Edition, Gennaro, ed. Mack Publishing Company:1990). The vaccines of the invention are administered, e.g., to human or non-human animal subjects, in order to stimulate an immune response specifically against the antigen and preferably to engender immunological memory that leads to mounting of a protective immune response should the subject encounter that antigen at some future time.

The vaccines of the invention comprise at least one polypeptide TLR ligand identified by the method of the invention and at least one antigen, and optionally a pharmaceutically acceptable carrier. As used herein, the phrase “pharmaceutically acceptable” refers to molecular entities and compositions that are “generally regarded as safe”, e.g., that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Other suitable carriers include polypeptides (e.g., keyhole limpet hemocyanin (KLH)), liposomes, insoluble salts of aluminum (e.g. aluminum phosphate or aluminum hydroxide), polynucleotides, polyelectrolytes, and water soluble carriers (e.g. muramyl dipeptides). Water or aqueous solutions, such as saline solutions and aqueous dextrose and glycerol solutions, are preferably employed as carriers, particularly for injectable solutions. Suitable pharmaceutical carriers are described in Remington's Pharmaceutical Sciences, 18^{th} Edition, Gennaro, ed. (Mack Publishing Company:1990).

As discussed above, the vaccines of the invention combine both signals required for the induction of a potent antigen-specific adaptive immune response: an innate immune system signal (i.e. TLR signaling) and an antigen receptor signal. This combination of signals provides for the induction of a potent immune response without the use of convention adjuvants. Thus, in preferred embodiments, the vaccines of the invention are formulated without conventional adjuvants. However, the invention also contemplates vaccines comprising at least one polypeptide TLR ligand identified by the method of the invention and at least one antigen, wherein the vaccine additionally comprises an adjuvant. As used herein, the term “adjuvant” refers to a compound or mixture that enhances the immune response to an antigen. An adjuvant can serve as a tissue depot that slowly releases the antigen and also as a lymphoid system activator that non-specifically enhances the immune response (Hood et al., Immunology, Second Ed., 1984, Benjamin/Cummings: Menlo Park, Calif., p. 384). Adjuvants include, but are not limited to, complete Freund's adjuvant, incomplete Freund's adjuvant, saponin, mineral gels such as aluminum hydroxide, surface active substances such as lysolecithin, pluronic polyols, polyanions, peptides, oil or hydrocarbon emulsions, keyhole limpet hemocyanins, and potentially useful human adjuvants such as N-acetyl-muramyl-L-threonyl-D-isoglutamine (thr-MDP), N-acetyl-nor-muramyl-L-alanyl-D-isoglutamine, N-acetylmuramyl-L-alanyl-D-isoglutaminyl-L-alanine-2-(1′-2′-dipalmitoyl-sn-glycero-3-hydroxyphosphoryloxy)-ethylamine, BCG (bacille Calmette-Guerin), and Corynebacterium parvum. Where the vaccine is intended for use in human subjects, the adjuvant should be pharmaceutically acceptable.

For example, vaccine administration can be by oral, parenteral (intramuscular, intraperitoneal, intravenous (IV) or subcutaneous injection), transdermal (either passively or using iontophoresis or electroporation), or transmucosal (nasal, vaginal, rectal, or sublingual) routes of administration or using bioerodible inserts and can be formulated in dosage forms appropriate for each route of administration. Moreover, the administration may be by continuous infusion or by single or multiple boluses.

The vaccine formulations may include diluents of various buffer content (e.g., Tris-HCl, acetate, phosphate), pH and ionic strength; additives such as detergents and solubilizing agents (e.g., Tween 80, Polysorbate 80); anti-oxidants (e.g., ascorbic acid, sodium metabisulfite); preservatives (e.g., Thimersol, benzyl alcohol); bulking substances (e.g., lactose, mannitol); or incorporation of the material into particulate preparations of polymeric compounds such as polylactic acid, polyglycolic acid, etc. or into liposomes. Hylauronic acid may also be used. See, e.g., Remington's Pharmaceutical Sciences, 18^{th} Edition, Gennaro, ed. (Mack Publishing Company: 1990).

The vaccines may be formulated so as to control the duration of action of the vaccine in a therapeutic application. For example, controlled release preparations can be prepared through the use of polymers to complex or adsorb the vaccine. For example, biocompatible polymers include matrices of poly(ethylene-co-vinyl acetate) and matrices of a polyanhydride copolymer of a stearic acid dimer and sebacic acid (see, for example, Sherwood et al. Bio/Technology 1992; 10:1446). The rate of release of the vaccine from such a matrix depends upon the molecular weight of the construct, the amount of the construct within the matrix, and the size of dispersed particles. See, for example, Saltzman et al. Biophys. J. 1989; 55:163; Sherwood et al. Bio/Technology 1992; 10:1446; Ansel et al. Pharmaceutical Dosage Forms and Drug Delivery Systems, 5th Edition (Lea & Febiger 1990); and Remington's Pharmaceutical Sciences, 18^{th} Edition, Gennaro, ed. (Mack Publishing Company: 1990). The vaccine can also be conjugated to polyethylene glycol (PEG) to improve stability and extend bioavailability times (see, e.g., U.S. Pat. No. 4,766,106).

Contemplated for use herein are oral solid dosage forms, which are described generally in Remington's Pharmaceutical Sciences, 18^{th} Edition, Gennaro, ed. (Mack Publishing Company: 1990) at Chapter 89, which is herein incorporated by reference. Solid dosage forms include tablets, capsules, pills, troches or lozenges, cachets, pellets, powders, or granules. Also, liposomal or proteinoid encapsulation may be used to formulate the present compositions (as, for example, proteinoid microspheres reported in U.S. Pat. No. 4,925,673). Liposomal encapsulation may be used and the liposomes may be derivatized with various polymers (e.g., U.S. Pat. No. 5,013,556). A description of possible solid dosage forms for a therapeutic is given by, for example, Marshall, K. In: Modern Pharmaceutics. Banker and Rhodes, eds. Chapter 10, 1979, herein incorporated by reference. In general, the formulation will include the therapeutic agent and inert ingredients which allow for protection against the stomach environment, and for release of the biologically active material in the intestine.

Also contemplated for use herein are liquid dosage forms for oral administration, including pharmaceutically acceptable emulsions, solutions, suspensions, and syrups, which may contain other components including inert diluents, wetting agents, emulsifying and/or suspending agents, and sweetening, flavoring, coloring, and/or perfuming agents.

For oral formulations, the location of release may be the stomach, the small intestine (the duodenum, the jejunem, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the therapeutic agent or by release of the therapeutic agent beyond the stomach environment, such as in the intestine. To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

A coating or mixture of coatings can also be used on tablets, which are not intended for protection against the stomach. These coatings can include sugar coatings, or coatings which make the tablet easier to swallow. Capsules may consist of a hard shell (such as gelatin) for delivery of dry therapeutic (i.e. powder). For liquid forms a soft gelatin shell may be used. The shell material of cachets could be thick starch or other edible paper. For pills, lozenges, molded tablets or tablet triturates, moist massing techniques can be used. The formulation of a material for capsule administration could also be as a powder, lightly compressed plugs, or even as tablets. These therapeutics could be prepared by compression.

One may dilute or increase the volume of the therapeutic agent with an inert material. These diluents could include carbohydrates, especially mannitol, α-lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic agent into a solid dosage form. Materials used as disintegrants include but are not limited to starch (including the commercial disintegrant based on starch, Explotab), sodium starch glycolate, Amberlite, sodium carboxymethylcellulose, ultramylopectin, sodium alginate, gelatin, orange peel, acid carboxymethyl cellulose, natural sponge and bentonite. Disintegrants may also be insoluble cationic exchange resins. Powdered gums may be used as disintegrants and as binders, and can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Other binders include methyl cellulose (MC), ethyl cellulose (EC) and carboxymethyl cellulose (CMC). Polyvinyl pyrrolidone (PVP) and/or hydroxypropylmethyl cellulose (HPMC) may be used in alcoholic solutions to granulate a peptide (or derivative).

An antifrictional agent may be included in the formulation to prevent sticking during the formulation process. Lubricants may be used as a layer between the therapeutic agent and the die wall, and these can include, but are not limited to, stearic acid including its magnesium and calcium salts, polytetrafluoroethylene (PTFE), liquid paraffin, vegetable oils and waxes. Soluble lubricants may also be used, such as sodium lauryl sulfate, magnesium lauryl sulfate, polyethylene glycol of various molecular weights, and Carbowax 4000 and 6000.

Glidants that might improve the flow properties of the therapeutic agent during formulation and to aid rearrangement during compression may be added. The glidants may include starch, talc, pyrogenic silica and hydrated silicoaluminate.

To aid dissolution of the therapeutic agent into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. Nonionic detergents that may be included in the formulation as surfactants include lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants may be present in the formulation of the therapeutic agent either alone or as a mixture in different ratios.

Controlled release oral formulations may be desirable. The therapeutic agent may be incorporated into an inert matrix which permits release by either diffusion or leaching mechanisms, e.g., gums. Slowly degenerating matrices may also be incorporated into the formulation. Some enteric coatings also have a delayed release effect. Another form of a controlled release is by a method based on the Oros therapeutic system (Alza Corp.), i.e. the therapeutic agent is enclosed in a semipermeable membrane which allows water to enter and push agent out through a single small opening due to osmotic effects.

Other coatings may be used for the formulation. These include a variety of sugars which could be applied in a coating pan. The therapeutic agent could also be given in a film coated tablet and the materials used in this instance are divided into 2 groups. The first are the nonenteric materials and include methyl cellulose, ethyl cellulose, hydroxyethyl cellulose, methylhydroxy-ethyl cellulose, hydroxypropyl cellulose, hydroxypropyl-methyl cellulose, sodium carboxy-methyl cellulose, providone and the polyethylene glycols. The second group consists of the enteric materials that are commonly esters of phthalic acid. A mix of materials might be used to provide the optimum film coating. Film coating may be carried out in a pan coater or in a fluidized bed or by compression coating.

Vaccines according to this invention for parenteral administration include sterile aqueous or non-aqueous solutions, suspensions, or emulsions. Examples of non-aqueous solvents or vehicles are propylene glycol, polyethylene glycol, vegetable oils, such as olive oil and corn oil, gelatin, and injectable organic esters such as ethyl oleate. Such dosage forms may also contain adjuvants, preserving, wetting, emulsifying, and dispersing agents. They can also be manufactured using sterile water, or some other sterile injectable medium, immediately before use.

Regarding the dosage of the vaccines of the present invention, the ordinary skilled practitioner, considering the therapeutic context, age, and general health of the recipient, will be able to ascertain proper dosing. The selected dosage depends upon the desired therapeutic effect, on the route of administration, and on the duration of the treatment desired. The dosing schedule may vary, depending on the circulation half-life, and the formulation used.

The vaccines of the present invention may be administered in conjunction with one or more additional active ingredients, pharmaceutical compositions, or vaccines.

### Methods of Modulating TLR Signaling

The invention provides methods of modulating TLR signalling, comprising administering to a subject in need thereof a polypeptide TLR ligand or vaccine of the invention. In preferred embodiments, the subject is a mammal. In particularly preferred embodiments, the subject is a human.

Thus, a polypeptide TLR ligand or vaccine of the invention may be administered to subjects, e.g., mammals including humans, in order to modulate TLR signaling. For a discussion of TLR signaling and assays to detect modulation of TLR signaling see the section Characterization of the polypeptide encoded by a nucleic acid insert, above.

In such subjects, modulation of TLR signaling may be used to modulate an immune response in the subject. In particular, modulation of TLR signaling may be used to modulate an antigen-specific immune response in the subject, e.g., to engender immunological memory that leads to mounting of a protective immune response should the subject encounter that antigen at some future time. Modulation of an immune response in a subject can be measured by standard tests including, but not limited to, the following: detection of antigen-specific antibody responses, detection of antigen specific T-cell responses, including cytotoxic T-cell responses, direct measurement of peripheral blood lymphocytes; natural killer cell cytotoxicity assays (see, for example, Provinciali et al. J. Immunol. Meth. 1992; 155:19-24), cell proliferation assays (see, for example, Vollenweider et al. J. Immunol. Meth. 1992; 149:133-135), immunoassays of immune cells and subsets (see, for example, Loeffler et al. Cytom. 1992; 13:169-174 and Rivoltini et al. Can. Immunol. Immunother. 1992; 34:241-251), and skin tests for cell mediated immunity (see, for example, Chang et al. Cancer Res. 1993; 53:1043-1050). Various methods and analyses for measuring the strength of the immune system are well known in the art (see, for example, Coligan et al., eds. Current Protocols in Immunology, Vol. 1. Wiley & Sons: 2000). The invention also provides methods of modulating TLR signaling comprising contacting a cell, wherein the cell comprises a TLR, with a polypeptide TLR ligand identified using the methods of the invention. As used herein, a cell that comprises a TLR is any cell that contains a given TLR protein, including a cell that endogenously expresses the TLR; a cell that does not endogenously express the TLR but ectopically expresses the TLR; and a cell that endogenously expresses the TLR and ectopically expresses additional TLR. In preferred embodiments the cell is a mammalian cell. In particularly preferred embodiments, the cell is a mouse cell or a human cell. The cell may be a cell cultured in vitro or a cell in vivo.

For a discussion of determination of TLR expression status; known TLR2, 4, and 5 expressing and non-expressing cells; and the generation of TLR expressing cells see the section TLR^{lo} cells and TLR^{hi} cells, above.

### EXAMPLES

The present invention is next described by means of the following examples. However, the use of these and other examples anywhere in the specification is illustrative only, and in no way limits the scope and meaning of the invention or of any exemplified form. Likewise, the invention is not limited to any particular preferred embodiments described herein. Indeed, many modifications and variations of the invention may be apparent to those skilled in the art upon reading this specification, and can be made without departing from its spirit and scope. The invention is therefore to be limited only by the terms of the appended claims, along with the full scope of equivalents to which the claims are entitled.

In accordance with the present invention there may be employed conventional molecular biology, microbiology, protein expression and purification, antibody, and recombinant DNA techniques within the skill of the art. Such techniques are explained fully in the literature. See, e.g., DNA Cloning: A Practical Approach, Vol I and II (Glover ed.:1985); Oligonucleotide Synthesis (Gait ed.:1984); Nucleic Acid Hybridization (Hames & Higgins eds.:1985); Transcription And Translation (Hames & Higgins, eds.:1984); Animal Cell Culture (Freshney, ed.:1986); Immobilized Cells And Enzymes (IRL Press: 1986); Perbal, A Practical Guide To Molecular Cloning (1984); Ausubel et al., eds. Current Protocols in Molecular Biology, (John Wiley & Sons, Inc.: 1994); Sambrook et al. gMolecular Cloning: A Laboratoiy Manual, 3^{rd} Edition (Cold Spring Harbor Laboratory Press: 2001); Harlow and Lane. Using Antibodies: A Laboratory Manual (Cold Spring Harbor Laboratory Press: 1999); PCR Primer: A Laboratory Manual, 2^{nd} Edition. Dieffenbach and Dveksler, eds. (Cold Spring Harbor Laboratory Press: 2003); and Hockfield et al. Selected Methods for Antibody and Nucleic Acid Probes (Cold Spring Harbor Laboratory Press: 1993).

### Example 1

### Cell Lines Ectopically Expressing TLRs

### Materials and Methods

Generation of cell lines ectopically expressing TLRs: Parental “293.luc” cells, which are HEK293 (ATCC Accession # CRL-1573) that have been stably transfected with an NF-κB reporter gene vector containing tandem copies of the NF-κB consensus sequence upstream of a minimal promoter fused to the firefly luciferase gene (κB-LUC), were cultured at 37° C. under 5% CO₂ in standard Dulbecco's Modified Eagle Medium (DMEM; e.g., Gibco) with 10% Fetal Bovine Serum (FBS; e.g., Hyclone).

Parental “3T3.luc” cells, which are NIH3T3 cells (ATCC Accession # CRL-1658) that have been stably transfected with an NF-κB reporter gene vector containing tandem copies of the NF-κB consensus sequence upstream of a minimal promoter fused to the firefly luciferase gene (κB-LUC), were cultured at 37° C. under 5% CO₂ in DMEM (e.g., Gibco) with 10% FBS (e.g., Hyclone).

The following pUNO-TLR plasmids were obtained from Invivogen: human TLR2 (catalog #puno-htlr2), human TLR4 isoform a (catalog #puno-htlr4a), mouse TLR5 (catalog #puno-mtlr5), and human TLR5 (catalog #puno-htlr5). The following pDUO-CD14/TLR plasmids were obtained from Invivogen: human CD14 plus human TLR2 (catalog #pduo-hcd14/tlr2) and human CD14 plus human TLR2 (catalog #pduo-hcd14/tlr4). The pUNO-TLR and pDUO-CD14/TLR plasmids are optimized for the rapid generation of stable transformants and for high levels of expression.

The pUNO-TLR or pDUO-CD14/TLR plasmids were transfected into HEK293 and/or NIH3T3 cells lines using Lyovec (Invivogen), a cationic lipid-based transfection reagent. Transfected cells were cultured at 37° C. under 5% CO₂ in DMEM (e.g., Gibco) medium with 10% FBS (e.g., Hyclone) supplemented with blasticidin (10 μg/ml). Stably transfected, individual blasticidin-resistant clones were isolated. The cell lines thereby generated are listed in Table 4.

TABLE 4
HEK293 and NIH3T3 lines ectopically
expressing TLRs and CD14.

| Clone designation | Transfected constructs |
|---|---|
| 293.luc | — |
| 293.hTLR2 | pUNO-hTLR2, κB-LUC |
| 293.hTLR2.hCD14 | pDUO-hCD14/hTLR2, κB-LUC |
| 293.hTLR4 | pUNO-hTLR4a, κB-LUC |
| 293.hTLR4.hCD14 | pDUO-hCD14/hTLR4, κB-LUC |
| 293.hTLR5 | pUNO-hTLR5, κB-LUC |
| 3T3.luc | — |
| 3T3.mTLR5 | pUNO-mTLR5, κB-LUC |

“293” = HEK293 cells.
“3T3” = NIH3T3 cells.
h = human.
m = mouse.

Analysis of TLR expression in HEK293 and NIH3T3 cells: Individual blasticidin-resistant clones of transfected HBEK293 and NIH3T3 have been isolated and characterized by Western blot analysis or flow cytometric analysis using polyclonal antibodies to the appropriate TLR to select clones which over-express the desired receptor.

To prepare whole cell lysate (WCE) for Western Blot analysis, sub-confluent cultures in 10 mm dishes were washed with PBS at room temperature. The following steps were then performed on ice or at 4° C. using fresh, ice-cold buffers. Six hundred microliters of RIPA buffer (Santa Cruz Biotechnology Inc., catalog #sc-24948; RIPA buffer: 1×TBS, 1% NP-40, 0.5% Sodium deoxycholate, 0.1% SDS, protease inhibitor cocktail) was added to the culture plate and the contents gently rocked for 15 minutes at 4° C. The cells were then harvested by scraping with a cell scraper and the scraped lysate was transferred to a microcentrifuge tube. The plate was washed once with 0.3 ml of RIPA buffer and combined with first lysate. An aliquot of 10111 of 10 mg/ml PMSF (Santa Cruz Biotechnology Inc., catalog #sc-3597) stock was added and the lysate passed through a 21-gauge needle to shear the DNA. The cell lysate was incubated 30-60 minutes on ice. The cell lysate was microcentrifuged 10,000×g for 10 minutes at 4° C. The lysate supernatant was transferred to a new microfuge tube and the pellet discarded. A 10 μl aliquot of lysate supernatant was loaded onto 10% SDS-PAGE gels and electrophoreses was performed according to standard protocols. The proteins were either stained by Coommassie Blue or transferred from the gels to a nitrocellulose or PVDF membrane using an electroblotting apparatus (BIORAD) according to the manufacturer's protocols. The membrane was then blotted with rabbit anti-hTLR2 polyclonal antibody (Invivogen, catalog #ab-htlr2) and reacted with a secondary antibody, goat anti-rabbit IgG Fc (Pierce, catalog #31341).

For flow cytometric analysis, HEK293 cells were removed from culture and resuspended in FACS staining buffer (phosphate buffered saline (PBS) containing 2% bovine serum albumin (BSA) and 0.01% sodium azide). A total of 10⁵ cells were then stained in a volume of 100 μl with the biotin labeled monoclonal antibody to TLR4, clone HTA125 (BD Pharmingen, catalog #551975) for 30 minutes at 4° C. Cells were then washed 3 times and incubated with streptavidin-FITC conjugated secondary antibody (BD Pharmingen, catalog #554060). Following incubation at 4° C. for 30 minutes samples were washed 3× with FACS buffer and then fixed in phosphate buffer containing 3% paraformaldehyde. Samples were then analyzed on a FACScan cytometer (BD Pharmingen) and analyzed using CellQuest software.

### Results and Discussion

In order to identify and affinity select potent ligands for TLRs from a peptide library displayed on bacteriophage, it is essential to employ cell lines expressing the TLR of choice. HEK293 cells and NIH3T3 cells, which had been previously stably transfected with a κB-LUC reporter gene, were stably transfected with pUNO-TLR and pDUO-CD14/TLR plasmid constructs from Invivogen. Individual blasticidin-resistant clones were isolated and characterized by Western blot analysis or flow cytometric analysis using polyclonal antibodies to CD14 and/or the appropriate TLR to select clones which over-express the desired receptor. Using this strategy, we have generated cell lines over-expressing various TLRs and CD14 as summarized in Table 5.

TABLE 5
Expression of TLRs and CD14 in HEK293 and NIH3T3 cells.

| Clone | TLR2 | TLR4 | TLR5 | CD14 |
|---|---|---|---|---|
| 293.luc | − | + | + | + |
| 293.hTLR2 | + | + | + | + |
| 293.hTLR2.hCD14 | + | + | + | + |
| 293.hTLR4 | − | ++ | + | + |
| 293.hTLR4.hCD14 | − | ++ | + | + |
| 293.hTLR5 | − | + | ++ | + |
| 3T3.luc | + | + | + | NT |
| 3T3.mTLR5 | + | + | ++ | NT |

h = human.
m = mouse.
NT = not tested.

Please note that while HEK293 (ATCC Accession # CRL-1573) obtained from the ATCC do not express TLR4 or respond to LPS (a TLR4 ligand), the parental 293.luc cell line used here does express detectable amounts of TLR4. The reason for this difference between the two cells lines is presently unclear. Notably, however, 293.luc cells (like HEK293 cells) do not to respond to LPS, indicating that they do not contain functional TLR4 protein.

As discussed above, one of the shared pathways of TLR signaling results in the activation of the transcription factor NF-κB. Therefore, in the cell lines generated here, expression of the NF-κB-dependent reporter gene serves as an indicator of TLR signaling.

### Example 2

### Phage Display Library Construction

### Materials and Methods

Construction of biased peptide libraries (BPL): Libraries of phage displaying overlapping peptides (between 5 and 20 amino acids) spanning the entire region of Measles Virus hemagglutinin (HA, a TLR2 ligand), respiratory syncytial virus fusion protein (RSV F, a TLR4 ligand), or E. coli flagellin (fliC, a TLR5 ligand) are constructed. The nucleotide and amino acid sequences of measles HA (GenBank Accession # D28950) are set forth in SEQ ID NO: 29 and SEQ ID NO: 30, respectively. The nucleotide and amino acid sequences of RSV F (GenBank Accession # D00334) are set forth in SEQ ID NO: 31 and SEQ ID NO: 32, respectively. The nucleotide and amino acid sequences of E. coli fliC are set forth in SEQ ID NO: 33 and SEQ ID NO: 34, respectively.

To construct a library, synthetic oligonucleotides covering the entire coding region of the polypeptide of interest (e.g. RSV F) are converted to double-stranded molecules, digested with EcoRI and HindIII restriction enzymes, and ligated into the T7SELECT bacteriophage vector (Novagen). The ligation reactions are packaged in vitro and amplified by either the plate or liquid culture method (according to manufacturer's instructions). The amplified phages are titred (according to manufacturer's instructions) to evaluate the total number of independent clones present in the library. The amplified library will contain approximately 10²-10³ individual clones.

Construction of random peptide libraries (RPL): Libraries of phage displaying random peptides of from 5 to 30 amino acids in length are constructed essentially as described above for biased peptide libraries, but utilizing oligonucleotides of defined length and random sequences. It is generally recognized that the major constraints of phage display are the bias and diversity (or completeness) of the RPL. To circumvent the former problem, the RPLs ARE constructed with only 32 codons (e.g. in the form NNK or NNS where N=A/T/G/C; K=G/T; S=G/C), thus reducing the redundancy inherent in the genetic code from a maximum codon number of 64 to 32 by eliminating redundant codons. For example, a 6-amino acid residue library displaying all possible hexapeptides requires 32⁶ (=10⁹) unique clones and is thus considered a complete library. Assuming a practical upper limit of ˜10⁹-10¹⁰ clones, RPLs longer than 7 residues accordingly risk being incomplete. This is not a major concern, since a longer residue library may actually increase the effective library diversity and thus is more suitable for isolating new polypeptide TLR ligands. The constructed libraries have a minimum of 10⁹ individual clones.

Construction of cDNA libraries: Libraries of phage displaying bacterial-derived polypeptides ARE constructed as described above for biased peptide libraries using cDNA derived from the bacterial source of choice. In order to obtain bacterial cDNA, bacterial mRNA is isolated and reversed-transcribed into cDNA. A PCR-ready single-stranded cDNA library made from total RNA of E. coli strain C600 is commercially available (Qbiogene). 10-mer degenerate oligonucleotides are employed as universal primer to synthesize the second strand of the E. coli cDNA. The amplified products are size-selected (ranging from 500 bp to 2 kb), excised and eluted from 1% agarose gel, and ligated into the T7Select10-3b vector (Novagen), which can accommodate proteins up to 1200 amino acids in length.

Construction of constrained cyclic peptide libraries: Two constrained cyclic peptide phage display libraries whose variable regions possess the following amino acid structure: C—X₇—C (cyclic 7-mer) and C—X₁₀—C (cyclic 10-mer), where C is a cysteine and X is any residue, were created. For each library, the variable region was generated using an extension reaction.

Random oligonucleotides were ordered PAGE purified from The Midland Certified Reagent Company. An EcoRI restriction enzyme site on the 5′ end and a HindIII site on the 3′ end were included for cloning purposes. In addition, the 3′ end contained additional flanking nucleotides creating a “handle”.

For the cyclic 10-mer inserts the random oligonucleotide was 5′-CAT GCC CGG AAT T CC TGC NNK NNK NNK NNK NNK NNK NNK NNK NNK NNK TGC GGA GGA GGA T AA AAG CTT TCG AGA C-3′ (SEQ ID NO: 90).

For the cyclic 7-mer inserts the random oligonucleotide was 5′-CAT GCC CGG AAT TCC TGC NNK NNI NNK NNK NNK NNK NNI TGC GGA GGA GGA TAA AAG CTT TCG AGA C-3′ (SEQ ID NO: 91).

For both oligonucleotides the 5′ EcoRI and 3′ HindIII sites are indicated by underlining and the variable region of the insert and nucleotides encoding the flanking cysteine residues are in bold. Amino acids in the variable region are encoded by NNK, where N=A/T/G/C and K=G/T. This nucleotide configuration reduces the number of possible codons from 64 to 32 while preserving the relative representation of each amino acid. In addition, the NNK configuration reduces the number of possible stop codons from three to one.

A universal oligonucleotide, 5′-GTC TCG AAA GCT TTT ATC CTC C′3′ (SEQ ID NO: 28) containing a HindIII site (underlined) was ordered PAGE purified from The Midland Certified Reagent Company. This universal oligonucleotide was annealed to the 3′ “handle” serving as a primer for the extension reaction. The annealing reaction was performed as follows: 5 μg of random oligonucleotide were mixed with 3 molar equivalents of the universal primer in dH₂0 with 100 mM NaCl. The mixture was heated to 95° C. for two minutes in a heat block. After that time, the heat block was turned off and allowed to cool to room temperature.

The annealed oligonucleotides were then added to an extension reaction mediated by the Klenow fragment of DNA polymerase I (New England Biolabs). The extension reaction was performed at 37° C. for 10 minutes, followed by an incubation at 65° C. for 15 minutes to inactivate the Klenow. The extended duplex was digested with 50 U of both EcoRI (New England Biolabs) and Hind111 (New England Biolabs) for 2 hours at 37° C. The digested products were separated by polyacrylamide gel electrophoresis, the bands of the correct size were excised from the gel, placed in 500 μl of elution buffer (10 mM magnesium acetate, 0.1% SDS, 500 mM ammonium acetate) and incubated overnight, with shaking, at 37° C. The following day the eluted DNA was purified by phenol:chloroform extraction followed by a standard ethanol precipitation.

The purified insert was ligated into T7 Select Vector arms (Novagen; cat. # 70548), using 0.6 Weiss Units of T4 DNA ligase (New England Biolabs). The entire ligation reaction was added to T7 Packaging Extract as per manufacturer's protocol (Novagen; cat. #70014). Using the bacterial strain 5615 (Novagen), the titer of the initial library was determined by a phage plaque assay (Novagen; T7Select System). Both the 7-mer and 10-mer cyclic peptide libraries have 5×10⁸ individual clones which approaches the upper achievable limit of the phage display system.

### Results and Discussion

A variety of phage display libraries are constructed for use in the screening assay to identify novel polypeptide TLR ligands. Such libraries include: 1) biased peptide libraries, which may be used to identify functional peptide TLR ligands within known polypeptide sequences; 2) random peptide libraries, which may be used to identify functional TLR ligands among randomly generated peptide sequences of between 5 and 30 amino acids in length; and 3) cDNA libraries, which may be used to identify functional TLR ligands from a microorganism of choice, e.g., the bacterium E. coli; and contrained cyclic peptide libraries, which contain random peptide sequences whose 3-dimensional conformation is restricted by cyclization via di-sulfide bonds between flanking cysteine residues.

### Example 3

### Screening Assay for Peptide TLR Ligands

### Materials and Methods

Screening of phage display libraries by biopanning: Phage display libraries are screened for peptide TLR ligands according to the following procedure. The phage display library is incubated on an in vitro cultured monolayer of cells that express minimal amounts of the TLR of interest (TLR^{lo}) in order to reduce non-specific binding, and then transferred to an in vitro suspension culture of cells expressing the relevant TLR (TLR^{hi}) to capture phage with binding specificity for the target TLR. After several washes with PBS to remove phage remaining unbound to the TLR^{hi} cells, TLR^{hi} cell-bound phages are harvested by centrifugation. The TLR^{hi} cells with bound phage are incubated with E. coli (strain BLT5615) in order to amplify the phage. This process is repeated three or more times to yield a phage population enriched for high affinity binding to the target TLR.

In each round of biopanning, the harvested phage that are bound to TLR^{hi} cells can be titred prior to amplification, amplified, and then titred again prior to initiation of the next cycle of biopanning. In this way, it is possible to determine the percent (%) of input phage in each cycle that are ultimately harvested from the TLR^{hi} cells. This calculation provides a round-by-round measure of enrichment within the phage display library for phage that display TLR-binding peptides.

Individual phage clones from the enriched pool are isolated, e.g., via plaque formation in E. coli.

### Results and Discussion

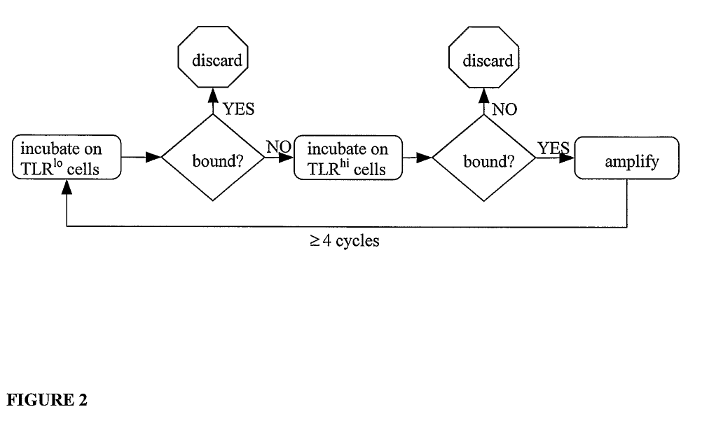

Phage display libraries are enriched for those clones that display peptides that specifically mediate TLR-binding by negative-positive panning as outlined in FIG. 2. Each cycle of panning consists of negative and positive panning as follows: the phage display library is incubated on a monolayer of cells that express minimal amounts of the TLR of interest (TLR^{lo}) in order to reduce non-specific binding; 2) the portion of the library that remains unbound to the monolayer of TLR^{lo} cells is transferred to a monolayer of cells expressing the relevant TLR (TLR^{hi}) to capture phage with binding specificity for the target TLR; 3) after several washes to remove phage remaining unbound to the monolayer of TLR^{hi} cells, bound phages are harvested by hypotonic shock of the cell monolayer; and 4) the harvested phage are amplified. This process is repeated three or more times to yield a phage population enriched for high affinity binding to the target TLR.

Individual phage clones from the enriched pool are isolated, e.g., via plaque formation in E. coli. These individual clones contain nucleotide sequences encoding for polypeptides that specifically bind to the TLR of choice.

### Example 4

### Screening Assay for Peptide TLR5 Ligands

### Materials and Methods

Generation of phage displaying a polypeptide TLR5 ligand: The coding region of the E. coli flagellin (fliC) gene (SEQ ID NO: 33) was cloned into the T7SELECT phage display vector (Novagen). Double stranded DNA encoding E. coli fliC was ligated to the T7Select 10-3 bacteriophage vector (Novagen). The ligation reactions were packaged in vitro and titred using the host E. coli strain BLR5615 that was grown in M9TB (Novagen). The recombinant phage was then amplified. Ligation, packaging, and amplification were performed according to manufacturer's instructions.

Generation of phage displaying an S-Tag polypeptide: The S-tag nucleotide sequence and amino acid sequences are set forth in SEQ ID NO: 35 and SEQ ID NO: 36, respectively. Double stranded DNA encoding the S-tag peptide sequence was ligated to the T7Select 10-3 bacteriophage vector (Novagen). The ligation reactions were packaged in vitro and titred using the host E. coli strain BLR5615 that was grown in M9TB (Novagen). The recombinant phage was then amplified. Ligation, packaging, and amplification were performed according to manufacturer's instructions. In order to simulate a random peptide library, 10³ fliC phages were mixed with 10¹⁰ S-tag phages (10⁻⁷ dilution).

NF-κB-dependent luciferase reporter assay: Parental 293 cells and 293.hTLR5 cells (see Example 1, above) were incubated with an aliquot of fliC-expressing T7SELECT phage, or S-tag expressing T7SELECT phage, for four to five hours at 37° C. As a negative control, cells were incubated with medium alone. NF-κB-dependent luciferase activity was measured using the Steady-Glo Luciferase Assay System by Promega (E2510), following the manufacturer's instructions. Luminescence was measured on a microplate luminometer (FARCyte, Amersham) and expressed as relative luminescence units (RLU) after subtracting the background reading obtained by exposing cells to the DMEM medium alone.

### Results and Discussion

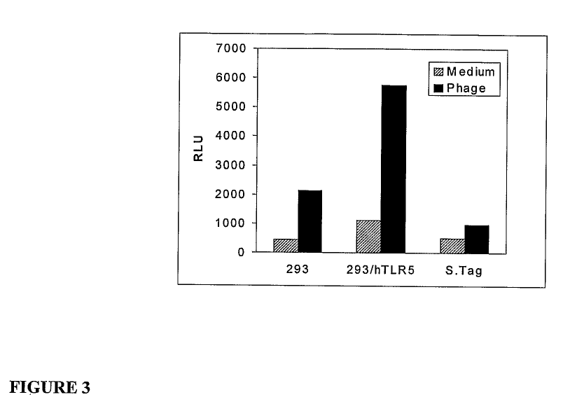

To verify the utility of the screening assay to identify TLR-binding polypeptides, we cloned the E. coli flagellin gene (fliC) into the T7SELECT phage display vector, expressed the protein in T7 phage, and examined binding of the recombinant fliC-phage to the cognate receptor, TLR5. The recombinant fliC-phage were incubated on parental HEK293 cells containing an NF-κB-dependent luciferase reporter construct (293) or on TLR5-overexpressing HEK293 cells containing an NF-κB-dependent luciferase reporter construct (293.hTLR5, see Example 1, above), and luciferase activity was measured. The data shown in FIG. 3 demonstrate that phage displaying fliC on their surface can bind to and activate TLR5. Moreover, the activation of the reporter gene correlates with over-expression of the appropriate TLR (i.e., TLR5).

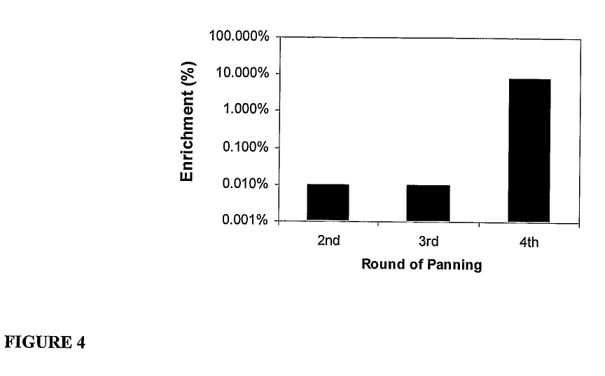

In order to simulate a random peptide library, 10³ fliC phages were mixed with 10¹⁰ control S-tag phages (10⁻⁷ dilution) and screened by biopanning as described in Example 3. For this screen, the TLR^{lo} cells were parental HEK293 (TLR5⁻) cells, and the TLR^{hi} cells were HEK293 cells ectopically expressing human TLR5 (293.hTLR5, see Example 1, above). Phage bound to 293.hTLR5 cells were harvested, titred, and amplified prior to initiation of each cycle of panning. In this way, it was possible to determine the % of input phage in each cycle that was ultimately harvested from the TLR5^{hi} cells. Results of the iterative negative-positive panning procedure are shown in FIG. 4. The data clearly show that it is feasible to isolate TLR5-binding phage by this biopanning strategy.

### Example 5

### Screening Assay for Peptide Tlr2 Ligands

### Materials and Methods

Construction of random peptide libraries (RPL): A pentameric random peptide phage display library of T7SELECT phage was constructed essentially as described in Example 2. A pair of phosphorylated oligonucleotides with the sequence NNBNNBNNBNNBNNB (where N=A/G/C/T, B=G/C/T) flanked at the 5′ and 3′ ends by EcoRI and HindIII sites, respectively, was synthesized. Equimolar amounts of the oligonucleotides were annealed by heating for 5 min at 90° C. with gradual cooling to 25° C. The double stranded DNA was ligated to T7Select 10-3 bacteriophage vector (Novagen) that had been previously digested with EcoRI and HindIII. The ligation reactions were packaged in vitro and titred using the host E. coli strain BLR5615 that was grown in M9TB (Novagen), generating 2.5×10⁷ clones, representing about 75% coverage of the library. The recombinant phage were subjected to several rounds of amplification to generate a total library of 1.35×10¹² phage, ensuring representation in excess of 5×10⁴ fold for each clone in the library.

Libraries of phage displaying random peptides 10, 15 and 20 amino acids in length were constructed essentially as described for the pentameric random peptide library, except that the phosphorylated oligonucleotides used were 30, 45, and 60 nucleotides in length, respectively.

Sequencing of phage inserts: Individual phage clones from the enriched pool are isolated via plaque formation in E. coli. The DNA inserts of individual phage are amplified in PCR using the commercially available primers T7SelectUP (5′-GGA GCT GTC GTA TTC CAG TC-3′; SEQ ID NO: 37; Novagen, catalog #70005) and T7SelectDOWN (5′-AAC CCC TCA AGA CCC GTT TA-3′; SEQ ID NO: 38; Novagen, catalog #70006). The PCR product DNA is purified using the QIAquick 96 PCR Purification Kit (Qiagen) and subjected to DNA sequencing using T7SelectUP and T7SelectDOWN primers.

Peptide synthesis: The synthetic monomer of the DPDSG motif, as well a concatemerized copy (DPDSG)₅ peptides were manufactured using solid phase synthesis methodologies and FMOC chemistry.

NF-κB-dependent luciferase reporter assay: Parental 293 cells and 293.hTLR2 cells (see Example 1, above) were incubated with an aliquot of test peptide four to five hours at 37° C. NF-κB-dependent luciferase activity was measured using the Steady-Glo Luciferase Assay System by Promega (E2510), following the manufacturer's instructions. Luminescence was measured on a microplate luminometer (FARCyte, Amersham) and expressed as relative luminescence units (RLU) after subtracting the background reading obtained by exposing cells to the DMEM medium alone.

### Results and Discussion

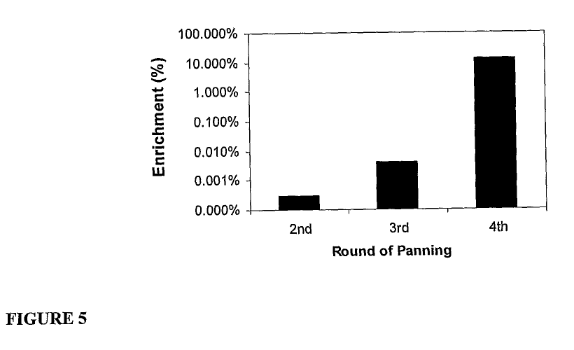

We constructed a pentameric random peptide phage display library in T7 phage. This phage library was then screened by biopanning as described in Example 3. For this screen, the TLR^{lo} cells were parental HEK293 (TLR2⁻) cells, and the TLR^{hi} cells were HEK293 cells ectopically expressing human TLR2 (293.hTLR2, see Example 1, above). Phage bound to 293.hTLR2 cells were harvested, titred, and amplified prior to initiation of the each cycle of panning. In this way, it was possible to determine the % of input phage in each cycle that was ultimately harvested from the TLR2^{hi} cells. FIG. 5 shows that the biopanning assay results in considerable enrichment after each iteration.

After 4 rounds of biopanning, individual phage clones from the enriched pool were isolated via plaque formation in E. coli. 10⁹ phage clones were randomly picked for sequencing. Of the 109 individual clones examined the motif DPDSG was predominant (see Tables 6 and 7). Homology search using tBLAST algorithm reveals that a majority (58%) of the novel sequences identified display a perfect match to various bacterial proteins of the database (See Table 6). Notable among these proteins are flagellin modification protein (FlmB) of Caulobacter crescentus, type 4 fimbrial biogenesis protein (PilX) of Pseudomonas, adhesin of Bordetella, and OmpA-related protein of Xantomonas. The rest of the sequences (42%) show no obvious homology to any known protein (See Table 7).

TABLE 6

Peptide TLR2 ligands which show identity to
known microbial proteins. % abundance =
percentage of all clones sequenced (n = 109)
having given peptide sequence.

| | | | |
|---|---|---|---|
| | SEQ | | |
| | ID | % | |
| PEPTIDE | NO | Abundance | Homology |

| | | | |
|---|---|---|---|
| DPDSG | 5 | 46.8 | flagellin modification protein FlmB |
| | | | of Caulobacter crescentus |
| IGRFR | 6 | 2.7 | Bacterial Type III secretion |
| | | | system protein |
| MGTLP | 7 | 1.8 | invasin protein of Salmonella |
| ADTHQ | 8 | 0.9 | Type 4 fimbrial biogenesis protein |
| | | | (PilX) of Pseudomonas |
| HLLPG | 9 | 0.9 | Salmonella SciJ protein |
| GPLLH | 10 | 0.9 | putative integral membrane protein |
| | | | of Streptomyces |
| NYRRW | 11 | 0.9 | membrane protein of Pseudomonas |
| LRQGR | 12 | 0.9 | adhesin of Bordetella pertusis |
| IMWFP | 13 | 0.9 | peptidase B of Vibrio cholerae |
| RVVAP | 14 | 0.9 | virulence sensor protein of |
| | | | Bordetella |
| IHVVP | 15 | 0.9 | putative integral membrane protein |
| | | | of Neisseria meningitidis |
| MFGVP | 16 | 0.9 | fusion of flagellar biosynthesis |
| | | | proteins FliR and FlhB of |
| | | | Clostridium |
| CVWLQ | 17 | 0.9 | outer membrane protein (porin) |
| | | | of Acinetobacter |
| IYKLA | 18 | 0.9 | flagellar biosynthesis protein, |
| | | | FlhF of Helicobacter |
| KGWF | 19 | 0.9 | ompA related protein of |
| | | | Xanthomonas |
| KYMPH | 20 | 0.9 | omp2a porin of Brucella |
| VGKND | 21 | 0.9 | putative porin/fimbrial assembly |
| | | | protein (LHrE) of Salmonella |
| THKPK | 22 | 0.9 | wbdk of Salmonella |
| SHIAL | 23 | 0.9 | Glycosyltransferase involved in LPS |
| | | | biosynthesis |
| AWAGT | 24 | 0.9 | Salmonella putative permease |

TABLE 7

Peptide TLR2 ligands which show no homology
to known proteins. % abundance = percentage
of all clones sequenced (n = 109) having given
peptide sequence.

| | | |
|---|---|---|
| PEPTIDE | SEQ ID NO | % ABUNDANCE |

| | | |
|---|---|---|
| NPPTT | 54 | 0.92% |
| MRRIL | 55 | 0.92% |
| MISS | 56 | 0.92% |
| RGGSK | 57 | 3.67% |
| RGGF | 58 | 0.92% |
| NRTVF | 59 | 0.92% |
| NRFGL | 60 | 0.92% |
| SRHGR | 61 | 0.92% |
| IMRHP | 62 | 0.92% |
| EVCAP | 63 | 0.92% |
| ACGVY | 64 | 0.92% |
| CGPKL | 65 | 0.92% |
| AGCFS | 66 | 0.92% |
| SGGLF | 67 | 0.92% |
| AVRLS | 68 | 0.92% |
| GGKLS | 69 | 0.92% |
| VSEGV | 70 | 3.67% |
| KCQSF | 71 | 0.92% |
| FCGLG | 72 | 0.92% |
| PESGV | 73 | 0.92% |

The biological activity of the TLR2-binding peptides isolated by the screening method was confirmed using the isolated peptides in an NF-κB-dependent reporter gene assay. For this assay, a synthetic monomer of the DPDSG motif (SEQ ID NO: 5), or a concatemerized copy (DPDSG)₅, was incubated on parental HEK293 cells containing an NF-κB-dependent luciferase reporter construct (293) and on TLR2-overexpressing HEK293 cells containing an NF-κB-dependent luciferase reporter construct (293.hTLR2, see Example 1, above). Luciferase activity was then measured. This assay showed that both the synthetic monomer of the DPDSG motif and the concatemerized copy (DPDSG)₅ activated luciferase reporter gene expression in a TLR2-dependent manner. Thus, the TLR2-binding peptides identified by the screening assay are functional peptide TLR2 ligands.

We also constructed 10, 15, and 20 amino acid random peptide phage display libraries in T7SELECT phage. These phage display libraries were pooled in equal proportion and then screened by biopanning as described in Example 3. For this screen, the TLR^{lo} cells were parental HEK293 (TLR2) cells, and the TLR^{hi} cells were HEK293 cells ectopically expressing human TLR2 and human CD14 (293.hTLR2.hCD14, see Example 1, above). After 4 rounds of biopanning, the enriched phage population was cloned by plaque formation in E. coli, and 96 clones were randomly picked for sequencing. Of the 96 clones analyzed three peptide sequences were particularly abundant (see Table 8). Homology search using tBLAST algorithm revealed that these peptide sequences show no obvious homology to any known protein. These novel peptide sequences share a common feature, in that all contain a high percentage (≧30%) of positively charged amino acids.

TABLE 8

Peptide TLR2 ligands which show no homology
to known proteins. % abundance = percentage
of all clones sequenced having given peptide
sequence.

| | SEQ | | POSITIVELY |
|---|---|---|---|
| | ID | % | CHARGED |
| PEPTIDE | NO | Abundance | AA (%) |
| KGGVGPVRRSSRLRRTTQPG | 25 | 33.3 | 6/20 (30%) |
| GRRGLCRGCRTRGRIKQLQSAHK | 26 | 16.1 | 9/23 (39%) |
| RWGYHLRDRKYKGVRSHKGVPR | 27 | 19.4 | 10/22 (45%) |

### Example 6

### In Vitro Transcription and Translation of the Novel TLR2 Polypeptide Ligands

### Materials and Methods

Generation of DNA inserts by PCR: Individual T7SELECT phage clones from the enriched pool are isolated via plaque formation in E. coli. The individual T7SELECT phage clones are dispensed in a 96-well plate, which serves as a master plate. Duplicate samples are subjected to PCR using phage specific primers, T7FOR (5′-GAA TTG TAA TAC GAC TCA CTA TAG GGA GGT GAT GAA GAT ACC CCA CC-3′; SEQ ID NO: 41), and T7REV (5′-TAA TAC GAC TCA CTA TAG GGC GAA GTG TAT CAA CAA GCT GG-3′; SEQ ID NO: 42) that flank the phage inserts. The forward primer is about 600 bp away from the insert and is designed to incorporate the T7 promoter upstream of the Kozak sequence (KZ), which is critical for optimal translation of eukaryotic genes, and a 6×HIS-tag sequence. The reverse primer includes the myc sequence at the c-terminus of the peptide. Therefore, the PCR product will contain all the signals necessary for optimal transcription and translation (T7 promoter, Kozak sequence and the ATG initiation codon), as well as and sequences encoding an N-terminal 6×HIS tag and a C-terminal myc tag for capture, detection and quantitation of the translated protein. The PCR products are purified using the QIAquick 96 PCR Purification Kit (Qiagen).

In vitro TNT: Rabbit reticulocyte lysate is programmed with the PCR DNA using TNT T7 Quick for PCR DNA kit (Promega), which couples transcription to translation. To initiate a TNT reaction, the DNA template is incubated at 30° C. for 60-90 min in the presence of rabbit reticulocyte lysate, RNA polymerase, amino acid mixture and RNAsin ribonuclease inhibitor.

Immunoanalysis of the in vitro translated protein: Immunoanalysis is used to confirm translation of the polypeptide TLR ligand. In these assays, an aliquot of the TNT reaction is analyzed by western blot using antibodies specific for one of the engineered tags, or by ELISA to allow normalization for protein levels across multiple samples. For a sandwich ELISA, 6×HIS-tagged protein is captured on Ni-NTA microplates and detected with an antibody to one of the heterologous tags (i.e., anti-c-myc).

NF-κB-dependent luciferase reporter assay: An aliquot of the in vitro synthesized peptide is monitored for the ability to activate an NF-κB-dependent luciferase reporter gene in cell lines expressing the target TLR. Cells stably transfected with an NF-κB luciferase reporter construct may constitutively express the appropriate TLR, or may be engineered to overexpress the TLR of choice. Cells seeded in a 96-well microplate are exposed to test peptide for four to five hours at 37° C. NF-κB-dependent luciferase activity is measured using the Steady-Glo Luciferase Assay System by Promega (E2510), following the manufacturer's instructions. Luminescence is measured on a microplate luminometer (FARCyte, Amersham). Specific activity of test compound is expressed as the EC₅₀, i.e., the concentration which yields a response that is 50% of the maximal response obtained with the appropriate control reagent, such as LPS. The EC₅₀ values are normalized to protein concentration as determined in the ELISA described above.

Dendritic cell activation assay: For this assay murine or human dendritic cell cultures are obtained. Murine DCs are generated in vitro as previously described (Lutz et al. J Immun Meth. 1999; 223:77-92). In brief, bone marrow cells from 6-8 week old C57BL/6 mice are isolated and cultured for 6 days in medium supplemented with 100 U/ml GMCSF (Granulocyte Macrophage Colony Stimulating Factor), replenishing half the medium every two days. On day 6, nonadherant cells are harvested and resuspended in medium without GMSCF and used in the DC activation assay. Human DCs are obtained commercially (Cambrex, Walkersville, Md.) or generated in vitro from peripheral blood obtained from healthy donors as previously described (Sallusto & Lanzavecchia. J Exp Med 1994; 179:1109-1118). In brief, peripheral blood mononuclear cells (PBMC) are isolated by Ficoll gradient centrifugation. Cells from the 42.5-50% interface are harvested and further purified following magnetic bead depletion of B- and T-cells using antibodies to CD19 and CD2, respectively. The resulting DC enriched suspension is cultured for 6 days in medium supplemented with 100 U/ml GMCSF and 1000 U/ml IL-4 (interleukin-4). On day 6, nonadherant cells are harvested and resuspended in medium without cytokines and used in the DC activation assay. An aliquot of the in vitro synthesized fusion protein is added to DC culture and the cultures are incubated for 16 hours. Supernatants are harvested, and cytokine (IFNγ, TNFα, IL-12 p70, IL-10 and IL-6) concentrations are determined by sandwich enzyme-linked immunosorbent assay (ELISA) using matched antibody pairs from BD Pharmingen or R&D Systems, following the manufacturer's instructions. Cells are harvested, and costimulatory molecule expression (e.g., B7-2) is determined by flow cytometry using antibodies from BD Pharmingen or Southern Biotechnology Associates following the manufacturer's instructions. Analysis is performed on a Becton Dickinson FACScan running Cellquest software.

Sequencing inserts of active phage: Those samples which test positive in the in vitro TNT cellular assays are traced back to the original master plate containing individual phage clones. The DNA inserts of positive clones are amplified in PCR using the primers T7FOR (5′-GAA TTG TAA TAC GAC TCA CTA TAG GGA GGT GAT GAA GAT ACC CCA CC-3′; SEQ ID NO: 43) and T7REV (5′-TAA TAC GAC TCA CTA TAG GGC GAA GTG TAT CAA CAA GCT GG-3′; SEQ ID NO: 44), or the commercially available primers T7SelectUP (5′-GGA GCT GTC GTA TTC CAG TC-3′; SEQ ID NO: 45; Novagen, catalog #70005) and T7SelectDOWN (5′-AAC CCC TCA AGA CCC GTT TA-3′; SEQ ID NO: 46; Novagen, catalog #70006). The DNA is purified and subjected to DNA sequencing using T7FOR and T7REV primers or T7SelectUP and T7SelectDOWN primers.

### Results and Discussion

We have performed in vitro TNT reactions on isolated T7SELECT phage expressing a novel polypeptide TLR2 ligand. The amount of protein produced by this method proved to be insufficient for detection in the TLR bioassays described above. Therefore, an alternate strategy, based upon ligase-independent cloning coupled with PCR from isolated phage expressing a novel polypeptide TLR2 ligand, was performed.

### Example 7

### Ligase Independent Cloning for In Vitro Analysis of Polypeptide TLR2 Ligand Activity

### Materials and Methods

Ligase independent cloning: Clones of T7Select 10-3 bacteriophage vector (Novagen) containing nucleic acid inserts encoding the polypeptide TLR2 ligands were subjected to PCR to isolate the nucleotide sequences encoding the TLR2-binding peptides. PCR was performed using the primers T7-LICf (5′-GAC GAC GAC AAG ATT GAG ACC ACT CAG AAC AAG GCC GCA CTT ACC GAC C-3′; SEQ ID NO: 74) and T7-LICr (5′-GAG GAG AAG CCC GGT CTA TTA CTC GAG TGC GGC CGC AAG-3′; SEQ ID NO: 75) at 10 pmol each with phage lysate at 1:20 dilution using the Taq polymerase master mix (Invitrogen) at 1:2 dilution. PCR cycling conditions were as follows: denaturation at 95° C. for 5 min; 30 cycles of denaturation step at 95° C. for 30 sec, annealing step at 58° C. for 30 sec, and extension at 72° C. for 30 sec; and a final extension at 72° C. for 10 min.

These sequences were then cloned into the pET-LIC24 and pMTBip-LIC vectors via ligase independent cloning (LIC). For LIC, an ˜800 bp PCR fragment, which includes a portion of the phage coat protein encoding sequence to facilitate expression and purification, was treated with T7 DNA polymerase in the presence of dATP and cloned into the linearized pET-LIC24 vector.

To construct the pET-LIC24 vector, an unique BseRI site was introduced into pET24a (Novagen). In order to introduce the BseRI site the 5′-phosphorylated primers pET24a-LICf (5′-TAT GCA TCA TCA CCA TCA CCA TGA TGA CGA CGA CAA GAG CCC GGG CTT CTC CTC AGC-3′; SEQ ID NO: 76) and pET24a-LIC-r (5′-TCA GCT GAG GAG AAG CCC GGG CTC TTG TCG TCG TCA TCA TGG TGA TGG TGA TGA TGC A-3′; SEQ ID NO: 77) were annealed and cloned into NdeI and Bpul 1021 digested pET24a via cohesive end ligation. The resulting construct was then digested with BseRI and treated with T4 DNA polymerase in the presence of dTTP to generate pET-LIC24 vector.

pMT-Bip-LIC was constructed in the same way as pET-LIC24 by inserting an annealed oligo into BglII and MluI digested vector pMTBip/V5-H isA. (Invitrogen). The annealed oligo was made using the 5′-phosphorylated primers pMTBip-LICf (5′-GAT CTC ATC ATC ACC ATC ACC ATG ATG ACG ACG ACA AGA GCC CGG GCT TCT CCT CAA-3′; SEQ ID NO: 78) and pMTBip-LICr (5′-CGC GTT GAG GAG AAG CCC GGG CTC TTG TCG TCG TCA TCA TGG TGA TGG TGA TGA TGA-3′; SEQ ID NO: 79).

Protein expression in E. coli: E. coli strain BLR (DE3) pLysS strain (Invitrogen) is transformed with pET-LIC plasmid DNA using a commercially available kit (Qiagen). A colony is inoculated into 2 mL LB containing 100 μg/ml carbenicillin, 34 μg/ml chloramphenicol, and 0.5% glucose and grown overnight at 37° C. with shaking. A fresh 2 mL culture is inoculated with a 1:20 dilution of the overnight culture and grown at 37° C. for several hours until OD₆₀₀=0.5-0.8. Protein expression is induced by the addition of IPTG to 1 mM for 3 hours.

Ni-NTA protein purification: E. coli cells transformed with the construct of interest were grown and induced as described above. The cells were harvested by centrifugation (7000 rpm×7 minutes in a Sorvall RC5C centrifuge) and the pellet re-suspended in lysis Buffer B (100 mM NaH2PO4, 10 mM Tris-HCl, 8 M urea, pH 8 adjusted with NaoH) and 10 mM imidazol. The suspension was freeze-thawed 4 times in a dry ice bath. The cell lysate was centrifuged (40,000 g for one hour in a Beckman Optima L ultracentrifuge) to separate the soluble fraction from inclusion bodies. The supernatant was mixed with 1 ml Ni-NTA resin (Qiagen Ni-NTA) that had been equilibrated with buffer B and binding of the proteins was allowed to proceed at 4° C. for 2-3 hours on a roller. The material was then loaded unto a lcm-diameter column. The bound material was then washed 2 times with 30 mL wash buffer (Buffer B+20 mM imidazol). The proteins were eluted in two rounds with 3 mL elution buffer twice (Buffer B+250 mM imidazol). The eluates were combined and the pools were used to perform a serial dialysis starting with 1 L of buffer (Buffer B+250 mM imidazol:2×PBS in a ratio of 1:1) with change in buffer every 4-8 hours. The final dialysis step was performed with two changes of PBS overnight. The integrity of the proteins was verified by SDS-PAGE and immunoblot.

Greater than 95% purity can be achieved. Optionally, to further reduce endotoxin contamination, the protein is chromatographed through Superdex 200 gel filtration in the presence of 1% deoxycholate to separate protein and endotoxin. A second round of Superdex 200 gel filtration in the absence of deoxycholate removes the detergent from the protein sample. Purified protein is concentrated and dialyzed against 1×PBS, 1% glycerol. The protein is aliquoted and stored at −80° C.

Protein expression in Drosophila S-2 cells: The pMTBip-LIC vectors are used to direct recombinant peptide expression in Drosophila S-2 cells. Conditioned medium from S-2 cells expressing the recombinant peptide may be directly used in bioassays to confirm the activity of the TLR-binding peptide. Drosophila S-2 cells and the Drosphila Expression System (DES) complete kit is obtained from Invitrogen (catalog#: K5120-01, K4120-01, K5130-1 and K4130-01). The growth and passaging of the S-2 cells, transfection and harvesting of the conditioned medium are performed according to manufacturer's protocol.

In vitro IL-8 assay: Parental 293 cells and 293.hTLR2.hCD14 cells (see Example 1, above) are seeded in 96-well microplates (50,000 cells/well), and aliquots of either purified recombinant peptide expressed in E. coli or conditioned medium from S-2 cells expressing recombinant peptide are added. As a positive control, parental 293 cells and 293.hTLR2.hCD14 cells are incubated with the PAMP tripalmitoyl-cystein-seryl-(lysyl)-3-lysine (Pam3Cys; e.g. Sigma-Aldrich). The microplates are then incubated overnight. The next day, the conditioned medium is harvested, transferred to a clean 96-well microplate, and frozen at −20° C. After thawing, the conditioned medium is assayed for the presence of IL-8 in a sandwich ELISA using an anti-human IL-8 matched antibody pair (Pierce, catalog #M801E and # M802B) following the manufacturer's instructions. Optical density is measured using a microplate spectrophotometer (FARCyte, Amersham).

### Results and Discussion

Clones of T7SELECT phage containing nucleic acid inserts encoding the peptide sequences of Table 9 were subjected to ligase independent cloning into a pET-LIC expression vector.

TABLE 9

Peptide TLR2 ligand sequences subjected to
ligase independent cloning (LIC) and
recombinantly expressed in E. coli.

| | | |
|---|---|---|
| | | Recombinant |
| PEPTIDE | SEQ ID NO | protein ID# |

| | | | |
|---|---|---|---|
| KGGVGPVRRSSRLRRTTQPG | 25 | ID#1 | > |
| GRRGLCRGCRTRGRIKQLQSAHK | 26 | ID#2 | |
| RWGYHLRDRKYKGVRSHKGVPR | 27 | ID#3 | |

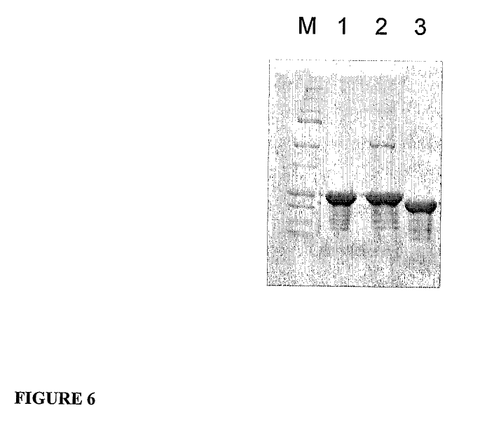

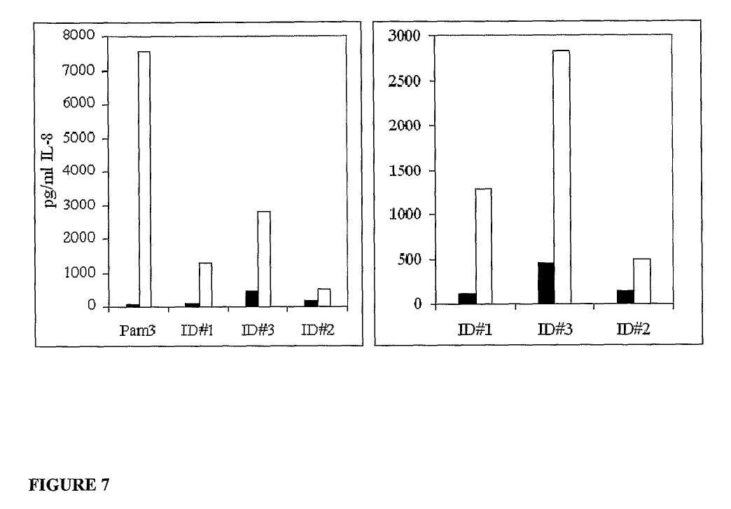

The pET-LIC vector was then used to direct recombinant peptide expression in E. coli host cells. The expressed peptides, which contain a His tag, were then purified on a Ni-NTA resin (see FIG. 6). These purified peptides were used in an IL-8 induction assay (see FIG. 7). The results of this assay clearly show that the novel polypeptides induce IL-8 production in a TLR2-dependent manner. Thus the polypeptides are functional peptide TLR2 ligands.

### Example 8

### A Polypeptide TLR2-Ligand:Listeria LLO-p60 Antigen Fusion Protein Vaccine

### Materials and Methods

Cloning of novel TLR ligands into E. coli: Double stranded DNA encoding the polypeptide TLR2 ligands is ligated upstream of sequences encoding a fusion protein of antigenic MHC class I and II epitopes of L. monocytogenes proteins LLO and p60. The amino acid sequence of the LLO-p60 fusion protein is given in SEQ ID NO: 39. These ligated sequences encoding a polypeptide TLR2 ligand:Listeria LLO-p60 antigen fusion protein are inserted into a plasmid expression vector. The expression construct is engineered by using convenient restriction enzyme sites or by PCR.

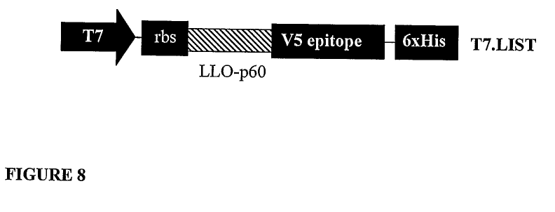

For example, sequences encoding the polypeptide TLR2 ligands are inserted upstream of the LLO-p60 encoding sequence in the expression construct T7.LIST (FIG. 8), where T7.LIST is assembled as described below. In this case, the expressed fusion protein will contain both a V5 epitope and a 6×His tag.

Generation of the T7.LIST plasmid: Sequences encoding the Listeria LLO-p60 antigen fusion protein are isolated as follows: First primers LLOF7 (5′-CTT AAA GAA TTC CCA ATC GAA AAG AAA CAC GCG GAT G-3′; SEQ ID NO: 47) and LLOR3 (5′-TTC TAC TAA TTC CGA GTT CGC TTT TAC GAG-3′; SEQ ID NO: 48) are used to amplify a 5′ portion of the LLO sequences. Next primers LLOF6 (5′-CTC GTA AAA GCG AAC TCG GAA TTA GTA GAA-3′; SEQ ID NO: 49) and P60R7 (5′ AGA GGT CTC GAG TGT ATT TGT TTT ATT AGC ATT TGT G-3′; SEQ ID NO: 50) are used to amplify the remaining fused 3′ portion LLO sequences and the p60 sequences. These two PCR fragments are then joined by a third PCR using the primers LLOF7 and P60R7. This PCR serves to mutate the LLO sequence spanned by LLOR3 and LLOF6 so as to remove the EcoRI site. This product is then ligated into the pCRT7CT-TOPO cloning vector (Invitrogen) to generate the T7.LIST plasmid. In this vector, the chimeric DNA insert is driven by the strong T7 promoter, and the insert is fused in frame to the V5 epitope (GKPIPNPLLGLDST; SEQ ID NO: 40) and polyhistidine (6×His) is located at the 3′ end of the gene (see FIG. 8).

Protein expression and immunoblot assay: In general, the following protocol is used to produce recombinant polypeptide TLR2 ligand:Listeria LLO-p60 antigen: fusion protein. E. coli strain BL (DE3) pLysS strain (Invitrogen) is transformed with the desired plasmid DNA using a commercially available kit (Qiagen). A colony is inoculated into 2 mL LB containing 100 μg/ml carbenicillin, 34 μg/ml chloramphenicol, and 0.5% glucose and grown overnight at 37° C. with shaking. A fresh 2 mL culture is inoculated with a 1:20 dilution of the overnight culture and grown at 37° C. for several hours until OD₆₀₀=0.5-0.8. Protein expression is induced by the addition of IPTG to 1 mM for 3 hours. The bacteria are harvested by centrifugation and the pellet is re-suspended in 100 μl of 1×SDS-PAGE sample buffer in the presence of β-mercaptoethanol. The samples are boiled for 5 minutes and 1/10 volume of each sample is loaded onto 10% SDS-PAGE gel and electrophoresed. The samples are transferred to PVDF membrane and probed with α-His antibody (Tetra His, Qiagen) at 1:1000 dilution followed by rabbit anti-mouse IgG/AP conjugate (Pierce) at 1:25,000. The immunoblot is developed using BCIP/NBT colometric assay kit (Promega).

Protein purification: Polypeptide TLR2 ligand:Listeria LLO-p60 antigen fusion proteins are expressed with a 6× Histidine tag to facilitate purification. E. coli cells transformed with the construct of interest are grown and induced as described above. Cells are harvested by centrifugation at 7,000 rpm for 7 minutes at 4° C. in a Sorvall RC5C centrifuge. The cell pellet is resuspended in Buffer A (6 M guanidine HCl, 100 mM NaH₂PO₄, 10 mM Tris-HCl, pH 8.0). The suspension can be frozen at −80° C. if necessary. Cells are disrupted by passing through a microfluidizer at 16,000 psi. The lysate is centrifuged at 30,000 rpm in a Beckman Coulter Optima LE-80K Ultracentrifuge for 1 hour. The supernatant is decanted and applied to Nickel-NTA resin at a ratio of 1 ml resin/1 L cell culture. The clarified supernatant is incubated with equilibrated resin for 2-4 hours by rotating. The resin is washed with 200 volumes of Buffer A. Non-specific protein binding is eliminated by subsequent washing with 200 volumes of Buffer B (8 M urea, 100 mM NaH₂PO₄, 10 mM Tris-HCl, pH 6.3). An additional 200 volume wash with buffer C (10 mM Tris-HCl, pH 8.0, 60% iso-propanol) reduces endotoxin to acceptable level (<0.1 EU/μg). Protein is eluted with Buffer D (8 M Urea, 100 mM NaH₂PO₄, 10 mM Tris-HCl, pH 4.5). Protein elution is monitored by SDS-PAGE or Western Blot (anti-His, anti-LLO and anti-p60). Greater than 95% purity can be achieved. Endotoxin level may be further reduced by chromatography through Superdex 200 gel filtration in the presence of 1% deoxycholate to separate protein and endotoxin. A second round of Superdex 200 gel filtration in the absence of deoxycholate removes the detergent from the protein sample. Purified protein is concentrated and dialyzed against 1×PBS, 1% glycerol. The protein is aliquoted and stored at −80° C.

Endotoxin assay: Endotoxin levels in recombinant fusion proteins are measured using the QCL-1000 Quantitative Chromogenic LAL test kit (BioWhittaker #50-648U), following the manufacturer's instructions for the microplate method.

Confirmation of TLR activity in NF-κB luciferase reporter assays: Purified recombinant polypeptide TLR2 ligand:Listeria LLO-p60 antigen fusion proteins are assayed for TLR activity and selectively in the NF-κB-dependent luciferase assay as described above.

Immunization: Recombinant polypeptide TLR2 ligand:Listeria LLO-p60 antigen fusion protein is suspended in phosphate-buffered saline (PBS), without exogenous adjuvant. BALB/c mice (n=10-20 per group) are immunized by s.c. injection at the base of the tail or in the hind footpad. Initial dosages tested range from 0.5 μg to 100 μg/animal. Positive control animals are immunized with 10³ CFU of live L. monocytogenes, while negative control animals receive mock-immunization with PBS alone.

Sublethal L. monocytogenes challenge: Seven days after immunization, BALB/c mice are infected by i.v. injection of 10³ CFU L. monocytogenes in 0.1 ml of PBS. Spleens and livers are removed 72 hours after infection and homogenized in 5 ml of sterile PBS+0.05% NP-40. Serial dilutions of the homogenates are plated on BHI agar. Colonies are enumerated after 48 hours of incubation. These experiments are performed a minimum of 3 times utilizing 10-20 animals per group. Mean bacterial burden per spleen or liver are compared between treatment groups by Student's t-Test.

Lethal L. monocytogenes challenge: Seven days after immunization, BALB/c mice are infected i.v. (10⁵ CFU) or p.o. (10⁹ CFU) with L. monocytogenes in 0.1 ml of PBS, and monitored daily until all animals have died or been sacrificed for humane reasons. Experiments are performed 3 times utilizing 10-20 animals per group. Mean survival times of different treatment groups are compared by Student's t-Test.

Induction of antigen-specific T-cell responses: CD8 T-cell responses are monitored at specific time points following vaccination (i.e. day 7, 14, 30, and 120) by quantitating the number of antigen-specific γ-interferon (IFNγ) secreting cells using ELISPOT (R&D Systems). At varying time points post-vaccination, T-cells are isolated from the draining lymph nodes and spleens of immunized animals and cultured in microtiter plates coated with capture antibody specific for the cytokine of interest. Synthetic peptides corresponding to the K^{d}-restricted epitopes p60₂₁₇₋₂₂₅ and LLO₉₁₋₉₉ are added to cultures for 16 hours. Plates are washed and incubated with anti-IFNγ detecting antibodies as directed by the manufacturer. Similarly, CD4 responses are quantified by IL-4 ELISPOT following stimulation with the I-A^{d} restricted CD4 epitopes LLO₁₈₉₋₂₀₀, LLO₂₁₆₋₂₂₇, and p60₃₀₀₋₃₁₁. Antigen specific responses are quantified using a dissection microscope with statistical analysis by Student's t-Test. For quantitation of CD8 responses, it is also possible to utilize flow cytometric analysis of T-cell populations following staining with recombinant MHC Class I tetramer (Beckman Coulter) loaded with the H-2^{d} restricted epitopes noted above.

Cytotoxic T-lymphocyte (CTL) responses: At specific time points following vaccination (i.e. day 7, 14, 30, and 120), induction of antigen-specific CTL activity is measured following in vitro restimulation of lymphoid cells from immune and control animals, using a modification of the protocol described by Bouwer and Hinrichs (see, for example, Bouwer and Hinrichs. Inf. Imm. 1996; 64:2515-2522). Briefly, erythrocyte-depleted spleen cells are cultured with Concanavalin A or peptide-pulsed, mitomycin C-treated syngeneic stimulator cells for 72 hours. Effector lymphoblasts are harvested and adjusted to an appropriate concentration for the effector assay. Effector cells are dispensed into round bottom black microtiter plates. Target cells expressing the appropriate antigen (e.g., cells infected with live L. monocytogenes or pulsed with p60 or LLO epitope peptides) are added to the effector cells to yield a final effector:target ratio of at least 40:1. After a four hour incubation, target cell lysis is determined by measuring the release of LDH using the CytoTox ONE fluorescent kit from Promega, following the manufacturer's instructions.

Antibody responses: Antigen-specific antibody titers are measured by ELISA according to standard protocols (see, e.g., Cote-Sierra et al. Infect Immun 2002; 70:240-248). For example, immunoglobulin isotype titers in the preimmune and immune sera are measured by using ELISA (Southern Biotechnology Associates, Inc., Birmingham, Ala.). Briefly, 96-well Nunc-Immuno plates (Nalge Nunc International, Roskilde, Denmark) are coated with 0.5 μg of COOHgp63 per well, and after exposure to diluted preimmune or immune sera, bound antibodies are detected with horseradish peroxidase-labeled goat anti-mouse IgG1 and IgG2a. ELISA titers are specified as the last dilution of the sample whose absorbance was greater than threefold the preimmune serum value. Alternatively, antigen-specific antibodies of different isotypes can be detected by Western blot analysis of sera against lysates of whole L. monocytogenes, using isotype-specific secondary reagents.

### Results and Discussion

L. monocytogenes is a highly virulent and prevalent food-borne gram-positive bacillus that causes gastroenteritis in otherwise healthy patients (Wing et al. J Infect Dis 2002; 185 Suppl 1:S18-S24), and more severe complications in immunocompromised patients, including meningitis, encephalitis, bacteremia and morbidity (Crum. Curr Gastroenterol Rep 2002; 4:287-296 and Frye et al. Clin Infect Dis 2002; 35:943-949). In vivo models have identified roles for both T- and B-cells in response to L. monocytogenes, with protective immunity attributed primarily to CD8 cytotoxic T cells (CTL) (Kersiek and Pamer. Curr Op Immunol 1999; 11:400-405). Studies during the past several years have led to the identification of several immunodominant L. monocytogenes epitopes recognized by CD4 and CD8 T-cells. In BALB/c mice, several peptides have been identified including the H-2K^{d} restricted epitopes LLO₉₁₋₉₉ and p60217225 (Pamer et al. Nature 1991; 353:852-854 and Pamer. J Immunol 1994; 152:686). The vaccine potential for such peptides is supported by studies demonstrating that the transfer of LLO₉₁₋₉₉-specific CTL into naïve hosts conveys protection to a lethal challenge with L. monocytogenes when the bacterial challenge is administered within a week of CTL transfer (Harty. J Exp Med 1992; 175:1531-1538). The mouse model of listeriosis (Geginat et al. J Immunol 1998; 160:6046-6055) has provided invaluable insights into the mechanisms of disease and the immunological response to infection with L. monocytogenes. This model allows the investigator to study both short-term and memory responses. This mouse model, with modifications, may be employed to confirm the in vivo efficacy and mechanism of action of novel polypeptide TLR ligands in fusion protein vaccines.

The polypeptide TLR2 ligands of the invention may be used to generate a fusion protein vaccine for Listeria infection. This vaccine comprises a fusion protein of a polypeptide TLR2 ligand and antigenic MHC class I and II epitopes of the L. monocytogenes proteins LLO and p60 (LLO-p60 fusion protein, SEQ ID NO: 39). The amino acid sequences of exemplary polypeptide TLR2 ligand:Listeria LLO-p60 antigen fusion proteins are set forth in SEQ ID NOs: 51, 52, and 53. For such vaccines, sequences encoding a polypeptide TLR2 ligand:Listeria LLO-p60 antigen fusion protein are inserted into a plasmid expression vector. The expression construct is then expressed in E. coli and the recombinant fusion protein purified based upon the included His tag.

The purified protein is then used to vaccinate mice. At specific time points following vaccination (i.e. day 7, 14, 30, and 120), animals are examined for antigen-specific humoral and cellular responses, including serum antibody titers, cytokine expression, CTL frequency and cytotoxicity activity, and antigen-specific proliferative responses. Protection versus Listeria infection is confirmed in the vaccinated animals using sublethal and lethal Listeria challenge assays. The polypeptide TLR2 ligand:Listeria LLO-p60 antigen fusion protein vaccine provides strong antigen-specific humoral and cellular immune responses, and provides protective immunity versus Listeria infection.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all values are approximate, and are provided for description.

Patents, patent applications, publications, product descriptions, and protocols are cited throughout this application, the disclosures of which are incorporated herein by reference in their entireties for all purposes.

1. A method to identify a polypeptide TLR ligand comprising:
a) providing a multiplicity of test phage in the form of a phage display library, wherein each individual test phage comprises a nucleic acid insert encoding a test polypeptide;
b) contacting a TLR^{lo} cell with the multiplicity of test phage;
c) retaining the test phage that do not bind to the TLR^{lo} cell;
d) contacting a TLR^{hi} cell, wherein the TLR is the same TLR as in step b), with the test phage retained in step c);
e) retaining the test phage that bind to the TLR^{hi} cell;
f) amplifying the test phage retained in step e);
g) optionally, repeating steps a) through f); and
h) characterizing the polypeptide encoded by the nucleic acid insert of a test phage amplified in step f),
wherein the polypeptide characterized in step h) is a polypeptide TLR ligand.

2. The method according to claim 1, wherein the steps a) through f) are performed at least 4 times.

3. The method according to claim 1, wherein the TLR is a mammalian TLR.

4. The method according to claim 1, wherein the TLR is TLR2, TLR4, or TLR5.

5. The method according to claim 1, wherein the TLR^{lo} cell and the TLR^{hi} cell are the same cell type.

6. The method according to claim 5, wherein the TLR^{lo} cell and the TLR^{hi} cell are both a HEK293 cell.

7. The method according to claim 5, wherein the TLR^{lo} cell and the TLR^{hi} cell are both an NIH3T3 cell.

8. The method according to claim 1, wherein the TLR^{lo} cell and the TLR^{hi} cell are both a mammalian cell.

9. The method according to claim 1, wherein step h) comprises:
i) determining the nucleic acid sequence of the nucleic acid insert; and
ii) using the nucleic acid sequence from step i) to deduce the amino acid sequence of the polypeptide encoded by the nucleic acid insert.

10. The method according to claim 1, wherein step h) comprises:
i) translating the nucleic acid insert to generate the polypeptide encoded by the nucleic acid insert; and
ii) characterizing said polypeptide.

11. The method according to claim 10, wherein step ii) comprises determining the amino acid sequence of the polypeptide.

12. The method according to claim 10, wherein step ii) comprises confirming the ability of the polypeptide to modulate TLR signaling.

13. A polypeptide TLR ligand identified by the method of claim 1.

14. A polypeptide comprising: i) a polypeptide TLR ligand identified by the method of claim 1; and ii) at least one antigen.

15. The polypeptide of claim 14, wherein the antigen is a polypeptide antigen.

16. The polypeptide of claim 14, wherein the antigen is selected from the group consisting of: a tumor-associated antigen, an allergen-related antigen, and a pathogen-related antigen.

17. (canceled)

18. (canceled)

19. The polypeptide of claim 16, wherein the pathogen-related antigen is an Influenza antigen, a Listeria monocytogenes antigen, a Dengue virus antigen, or a West Nile Virus antigen.

20. A method of modulating TLR signaling in a subject comprising administering to a subject in need thereof the polypeptide of claim 13 or 14.

21. The method of claim 20, wherein the subject is a mammal.

22. A method of modulating TLR signaling in a cell comprising contacting a cell, wherein the cell comprises the TLR, with the polypeptide of claim 13 or 14.

23. The method of claim 22, wherein the cell is a mammalian cell.

24. A vaccine comprising the polypeptide of claim 13 or 14 and a pharmaceutically acceptable carrier.

25. A vaccine comprising: i) a polypeptide TLR ligand identified by the method of claim 1; ii) at least one antigen; and iii) a pharmaceutically acceptable carrier.

26. The vaccine of claim 25, wherein the polypeptide TLR ligand and the antigen are covalently linked.

27. The vaccine of claim 25, wherein the antigen is a polypeptide antigen.

28. The vaccine of claim 25, wherein the antigen is selected from the group consisting of: a tumor-associated antigen, an allergen-related antigen, and a pathogen-related antigen.

29. (canceled)

30. (canceled)

31. The vaccine of claim 28, wherein the pathogen-related antigen is an Influenza antigen, a Listeria monocytogenes antigen, a Dengue virus antigen, or a West Nile Virus antigen.

32. A method of modulating TLR signaling in a subject comprising administering to a subject in need thereof the vaccine of claim 24.

33. The method of claim 32, wherein the subject is a mammal.


SEQUENCE LISTING (91 sequences)

SEQ ID NO: 1 - DNA, 2838 bp, Mus musculus
ggcacgagagatggctagtgggcacggggagcggcggctggaggactcctaggctccgggcaggcggtcactggcaggagatgtgtccgcaatcatagtttctgatggtgaaggttggacggcagtctctgcgacctagaagtggaaaagatgtcgttcaaggaggtgcggactgtttccttctgaccaggatcttgtttctgagtgtaggggcttcacttctctgcttttcgttcatctctggagcatccgaattgcatcaccggtcagaaaacaacttaccgaaacctcagacaaagcgtcaaatctcagaggatgctacgagctctttggctcttctggatcttggtggccataacagtcctcttcagcaaacgctgttctgctcaggagtctctgtcatgtgatgcttctggggtgtgtgatggccgctccaggtctttcacctctattccctccggactcacagcagccatgaaaagccttgacctgtctttcaacaagatcacctacattggccatggtgacctccgagcgtgtgcgaacctccaggttctgattttgaagtccagcagaatcaatacaatagagggagacgccttttattctctgggcagtcttgaacatttggatttgtctgataatcacctatctagtttatcttcctcctggttcgggcccctttcctctttgaaatacttaaacttaatgggaaatccttaccagacactgggggtaacatcgctttttcccaatctcacaaatttacaaaccctcaggataggaaatgtagagactttcagtgagataaggagaatagattttgctgggctgacttctctcaatgaacttgaaattaaggcattaagtctccggaattatcagtcccaaagtctaaagtcgatccgcgacatccatcacctgactcttcacttaagcgagtctgctttcctgctggagatttttgcagatattctgagttctgtgagatatttagaactaagagatactaacttggccaggttccagttttcaccactgcccgtagatgaagtcagctcaccgatgaagaagctggcattccgaggctcggttctcactgatgaaagctttaacgagctcctgaagctgttgcgttacatcttggaactgtcggaggtagagttcgacgactgtaccctcaatgggctcggcgatttcaacccctcggagtcagacgtagtgagcgagctgggtaaagtagaaacagtcactatccggaggttgcatatcccccagttctatttgttttatgacctgagtactgtctattccctcctggagaaggtgaagcgaatcacagtagagaacagcaaggtcttcctggttccctgctcgttctcccagcatttaaaatcattagaattcttagacctcagcgaaaatctgatggttgaagaatatttgaagaactcagcctgtaagggagcctggccttctctacaaaccttagttttgagccagaatcatttgagatcaatgcaaaaaacaggagagattttgctgactctgaaaaacctgacctcccttgacatcagcaggaacacttttcatccgatgcccgacagctgtcagtggccagaaaagatgcgcttcctgaatttgtccagtacagggatccgggtggtaaaaacgtgcattcctcagacgctggaggtgttggatgttagtaacaacaatcttgactcattttctttgttcttgcctcggctgcaagagctctatatttccagaaataagctgaaaacactcccagatgcttcgttgttccctgtgttgctggtcatgaaaatcagagagaatgcagtaagtactttctctaaagaccaacttggttcttttcccaaactggagactctggaagcaggcgacaaccactttgtttgctcctgcgaactcctatcctttactatggagacgccagctctggctcaaatcctggttgactggccagacagctacctgtgtgactctccgcctcgcctgcacggccacaggcttcaggatgcccggccctccgtcttggaatgtcaccaggctgcactggtgtctggagtctgctgtgcccttctcctgttgatcttgctcgtaggtgccctgtgccaccatttccacggactgtggtacctgagaatgatgtgggcgtggctccaggccaagaggaagcccaagaaagctccctgcagggacgtttgctatgatgcctttgtttcctacagtgagcaggattcccattgggtggagaacctcatggtccagcagctggagaactctgacccgccctttaagctgtgtctccacaagcgggacttcgttccgggcaaatggatcattgacaacatcatcgattccatcgaaaagagccacaaaactgtgttcgtgctttctgagaacttcgtacggagcgagtggtgcaagtacgaactggacttctcccacttcaggctctttgacgagaacaacgacgcggccatccttgttttgctggagcccattgagaggaaagccattccccagcgcttctgcaaactgcgcaagataatgaacaccaagacctacctggagtggcccttggatgaaggccagcaggaagtgttttgggtaaatctgagaactgcaataaagtcctaggttctccacccagttcctgacttccttaactaaggtctttgtgacacaaactgtaacaaagtttataagtaacatagaattgtattattgaggatattaactatgggttttgtcttgaatactgttatataaatatgtgacatcaggaaaaaaaaaaaaaaaaaaaaa

SEQ ID NO: 2 - protein, 784 aa, Mus musculus
MLRALWLFWILVAITVLFSKRCSAQESLSCDASGVCDGRSRSFTSIPSGLTAAMKSLDLSFNKITYIGHGDLRACANLQVLILKSSRINTIEGDAFYSLGSLEHLDLSDNHLSSLSSSWFGPLSSLKYLNLMGNPYQTLGVTSLFPNLTNLQTLRIGNVETFSEIRRIDFAGLTSLNELEIKALSLRNYQSQSLKSIRDIHHLTLHLSESAFLLEIFADILSSVRYLELRDTNLARFQFSPLPVDEVSSPMKKLAFRGSVLTDESFNELLKLLRYILELSEVEFDDCTLNGLGDFNPSESDVVSELGKVETVTIRRLHIPQFYLFYDLSTVYSLLEKVKRITVENSKVFLVPCSFSQHLKSLEFLDLSENLMVEEYLKNSACKGAWPSLQTLVLSQNHLRSMQKTGEILLTLKNLTSLDISRNTFHPMPDSCQWPEKMRFLNLSSTGIRVVKTCIPQTLEVLDVSNNNLDSFSLFLPRLQELYISRNKLKTLPDASLFPVLLVMKIRENAVSTFSKDQLGSFPKLETLEAGDNHFVCSCELLSFTMETPALAQILVDWPDSYLCDSPPRLHGHRLQDARPSVLECHQAALVSGVCCALLLLILLVGALCHHFHGLWYLRMMWAWLQAKRKPKKAPCRDVCYDAFVSYSEQDSHWVENLMVQQLENSDPPFKLCLHKRDFVPGKWIIDNIIDSIEKSHKTVFVLSENFVRSEWCKYELDFSHFRLFDENNDAAILVLLEPIERKAIPQRFCKLRKIMNTKTYLEWPLDEGQQEVFWVNLRTAIKS

SEQ ID NO: 3 - DNA, 2756 bp, Homo sapiens
gagaaagcgcagccaggcggctgctcggcgttctctcaggtgactgctcggagttctcccagtgtttggtgttgcaagcaggatccaaaggagacctatagtgactcccaggagctcttagtgaccaagtgaaggttgaagcactggacaatgccacatactttgtggatggtgtgggtcttgggggtcatcatcagcctctccaaggaagaatcctccaatcaggcttctctgtcttgtgaccgcaatggtatctgcaagggcagctcaggatctttaaactccattccctcagggctcacagaagctgtaaaaagccttgacctgtccaacaacaggatcacctacattagcaacagtgacctacagaggtgtgtgaacctccaggctctggtgctgacatccaatggaattaacacaatagaggaagattctttttcttccctgggcagtcttgaacatttagacttatcctataattacttatctaatttatcgtcttcctggttcaagcccctttcttctttaacattcttaaacttactgggaaatccttacaaaaccctaggggaaacatctcttttttctcatctcacaaaattgcaaatcctgagagtgggaaatatggacaccttcactaagattcaaagaaaagattttgctggacttaccttccttgaggaacttgagattgatgcttcagatctacagagctatgagccaaaaagtttgaagtcaattcagaacgtaagtcatctgatccttcatatgaagcagcatattttactgctggagatttttgtagatgttacaagttccgtggaatgtttggaactgcgagatactgatttggacactttccatttttcagaactatccactggtgaaacaaattcattgattaaaaagtttacatttagaaatgtgaaaatcaccgatgaaagtttgtttcaggttatgaaacttttgaatcagatttctggattgttagaattagagtttgatgactgtacccttaatggagttggtaattttagagcatctgataatgacagagttatagatccaggtaaagtggaaacgttaacaatccggaggctgcatattccaaggttttacttattttatgatctgagcactttatattcacttacagaaagagttaaaagaatcacagtagaaaacagtaaagtttttctggttccttgtttactttcacaacatttaaaatcattagaatacttggatctcagtgaaaatttgatggttgaagaatacttgaaaaattcagcctgtgaggatgcctggccctctctacaaactttaattttaaggcaaaatcatttggcatcattggaaaaaaccggagagactttgctcactctgaaaaacttgactaacattgatatcagtaagaatagttttcattctatgcctgaaacttgtcagtggccagaaaagatgaaatatttgaacttatccagcacacgaatacacagtgtaacaggctgcattcccaagacactggaaattttagatgttagcaacaacaatctcaatttattttctttgaatttgccgcaactcaaagaactttatatttccagaaataagttgatgactctaccagatgcctccctcttacccatgttactagtattgaaaatcagtaggaatgcaataactacgttttctaaggagcaacttgactcatttcacacactgaagactttggaagctggtggcaataacttcatttgctcctgtgaattcctctccttcactcaggagcagcaagcactggccaaagtcttgattgattggccagcaaattacctgtgtgactctccatcccatgtgcgtggccagcaggttcaggatgtccgcctctcggtgtcggaatgtcacaggacagcactggtgtctggcatgtgctgtgctctgttcctgctgatcctgctcacgggggtcctgtgccaccgtttccatggcctgtggtatatgaaaatgatgtgggcctggctccaggccaaaaggaagcccaggaaagctcccagcaggaacatctgctatgatgcatttgtttcttacagtgagcgggatgcctactgggtggagaaccttatggtccaggagctggagaacttcaatccccccttcaagttgtgtcttcataagcgggacttcattcctggcaagtggatcattgacaatatcattgactccattgaaaagagccacaaaactgtctttgtgctttctgaaaactttgtgaagagtgagtggtgcaagtatgaactggacttctcccatttccgtctttttgatgagaacaatgatgctgccattctcattcttctggagcccattgagaaaaaagccattccccagcgcttctgcaagctgcggaagataatgaacaccaagacctacctggagtggcccatggacgaggctcagcgggaaggattttgggtaaatctgagagctgcgataaagtcctaggttcccatatttaagaccagtctttgtctagttgggatctttatgtcactagttatagttaagttcattcagacataattatataaaaactacgtggatgtaccgtcatttgaggacttgcttactaaaactacaaaacttcaaattttgtctggggtgctgttttataaacatatgccagatttaaaaaaaaaaaaaaaaaaaaaaaaaaaaaaaaaaaaaaaaaaaaaaaaaaaaaaaaaaaaaaaaaa

SEQ ID NO: 4 - protein, 784 aa, Homo sapiens
MPHTLWMVWVLGVIISLSKEESSNQASLSCDRNGICKGSSGSLNSIPSGLTEAVKSLDLSNNRITYISNSDLQRCVNLQALVLTSNGINTIEEDSFSSLGSLEHLDLSYNYLSNLSSSWFKPLSSLTFLNLLGNPYKTLGETSLFSHLTKLQILRVGNMDTFTKIQRKDFAGLTFLEELEIDASDLQSYEPKSLKSIQNVSHLILHMKQHILLLEIFVDVTSSVECLELRDTDLDTFHFSELSTGETNSLIKKFTFRNVKITDESLFQVMKLLNQISGLLELEFDDCTLNGVGNFRASDNDRVIDPGKVETLTIRRLHIPRFYLFYDLSTLYSLTERVKRITVENSKVFLVPCLLSQHLKSLEYLDLSENLMVEEYLKNSACEDAWPSLQTLILRQNHLASLEKTGETLLTLKNLTNIDISKNSFHSMPETCQWPEKMKYLNLSSTRIHSVTGCIPKTLEILDVSNNNLNLFSLNLPQLKELYISRNKLMTLPDASLLPMLLVLKISRNAITTFSKEQLDSFHTLKTLEAGGNNFICSCEFLSFTQEQQALAKVLIDWPANYLCDSPSHVRGQQVQDVRLSVSECHRTALVSGMCCALFLLILLTGVLCHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKS

SEQ ID NO: 5 - protein, 5 aa, artificial
  synthetic peptide
DPDSG

SEQ ID NO: 6 - protein, 5 aa, artificial
  synthetic peptide
IGRFR

SEQ ID NO: 7 - protein, 5 aa, artificial
  synthetic peptide
MGTLP

SEQ ID NO: 8 - protein, 5 aa, artificial
  synthetic peptide
ADTHQ

SEQ ID NO: 9 - protein, 5 aa, artificial
  synthetic peptide
HLLPG

SEQ ID NO: 10 - protein, 5 aa, artificial
  synthetic peptide
GPLLH

SEQ ID NO: 11 - protein, 5 aa, artificial
  synthetic peptide
NYRRW

SEQ ID NO: 12 - protein, 5 aa, artificial
  synthetic peptide
LRQGR

SEQ ID NO: 13 - protein, 5 aa, artificial
  synthetic peptide
IMWFP

SEQ ID NO: 14 - protein, 5 aa, artificial
  synthetic peptide
RVVAP

SEQ ID NO: 15 - protein, 5 aa, artificial
  synthetic peptide
IHVVP

SEQ ID NO: 16 - protein, 5 aa, artificial
  synthetic peptide
MFGVP

SEQ ID NO: 17 - protein, 5 aa, artificial
  synthetic peptide
CVWLQ

SEQ ID NO: 18 - protein, 5 aa, artificial
  synthetic peptide
IYKLA

SEQ ID NO: 19 - protein, 4 aa, artificial
  synthetic peptide
KGWF

SEQ ID NO: 20 - protein, 5 aa, artificial
  synthetic peptide
KYMPH

SEQ ID NO: 21 - protein, 5 aa, artificial
  synthetic peptide
VGKND

SEQ ID NO: 22 - protein, 5 aa, artificial
  synthetic peptide
THKPK

SEQ ID NO: 23 - protein, 5 aa, artificial
  synthetic peptide
SHIAL

SEQ ID NO: 24 - protein, 5 aa, artificial
  synthetic peptide
AWAGT

SEQ ID NO: 25 - protein, 20 aa, artificial
  synthetic peptide
KGGVGPVRRSSRLRRTTQPG

SEQ ID NO: 26 - protein, 23 aa, artificial
  synthetic peptide
GRRGLCRGCRTRGRIKQLQSAHK

SEQ ID NO: 27 - protein, 22 aa, artificial
  synthetic peptide
RWGYHLRDRKYKGVRSHKGVPR

SEQ ID NO: 28 - DNA, 22 bp, artificial
  universal oligonucleotide
gtctcgaaagcttttatcctcc

SEQ ID NO: 29 - DNA, 1854 bp, Measles virus
atgtcaccacaacgagaccgaataaatgccttctacaaagacaacccccatcctaagggaagtaggatagttattaacagagaacatcttatgattgatagaccttatgttttgctggctgttctattcgtcatgtttctgagcttgatcgggttgctagccattgcaggcattagacttcatcgggcagccatctacactgcagagatccataaaagcctcagcaccaatctagatgtaactaactcaatcgagcatcaggtcaaggacgtgctgacaccactcttcaagatcatcggtgatgaagtgggcctgaggacacctcagagattcactgacctagtgaaattcatctctgacaagattaaattccttaatccggatagggagtacgacttcagggatctcacttggtgtatcaacccgccagagagaatcaaattggattatgatcaatactgtgcagatgtggctgctgaagaactcatgaatgcattggtgaacgcaactctactggaggccagggcaaccaatcagttcctagctgtctcaaagggaaactgctcagggcccactacaatcagaggtcaattctcaaacatgtcgctgtccctgttggacttgtatttaagtcgaggttacaatgtgtcatctatagtcactatgacatcccagggaatgtacgggggaacttacctagtggaaaagcctaatctgagcagtaaagggtcagagttgtcacaactgagcatgcaccgagtgtttgaagtaggtgtgatcagaaatccgggtttgggggctccggtgttccatatgacaaactattttgagcaatcagtcagtaatgatttcagcaactgcatggtggctttaggggagctcaaattcgcagccctctgtcacagggaagattctatcacaattccctatcaggggtcagggaaaggtgtcagcttccagctcgtcaagctaggtgtctggaaatccccaaccgacatgcaatcctgggtccccctatcaacggatgatccagtgatagataggctttacctctcatctcacagaggtgttatcgctgacaatcaagcaaaatgggctgtcccgacaacacggacagatgacaagttgcgaatggagacatgcttccagcaggcgtgtaagggcaaaatccaagcactctgcgagaatcccgagtgggcaccactgaaggacaacaggattccttcatacggggtcttgtctgttaatctgagtctgacagttgagctcaaaatcaaaattgcttcaggattcgggccattgatcacacacggttcagggatggacctatacaaatccaaccacaacaatgtgtattggctgaccatcccgccaatgaagaacctagccttaggtgtaatcaacacattagagtggataccgagattcaaggttagtcccaacctcttcactgttccaatcaaggaagcaggcgaggactgccatgccccaacatacctacctgcggaggtggatggtgatgtcaaactcagttccaatctggtgattctacctggtcaagatctccaatatgttttggcaacctacgatacttccagggttgaacatgctgtggtttattatgtttacagcccgggccgctcattttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcactcgggaagatggaaccaataccagatag

SEQ ID NO: 30 - protein, 617 aa, Measles virus
MSPQRDRINAFYKDNPHPKGSRIVINREHLMIDRPYVLLAVLFVMFLSLIGLLAIAGIRLHRAAIYTAEIHKSLSTNLDVTNSIEHQVKDVLTPLFKIIGDEVGLRTPQRFTDLVKFISDKIKFLNPDREYDFRDLTWCINPPERIKLDYDQYCADVAAEELMNALVNATLLEARATNQFLAVSKGNCSGPTTIRGQFSNMSLSLLDLYLSRGYNVSSIVTMTSQGMYGGTYLVEKPNLSSKGSELSQLSMHRVFEVGVIRNPGLGAPVFHMTNYFEQSVSNDFSNCMVALGELKFAALCHREDSITIPYQGSGKGVSFQLVKLGVWKSPTDMQSWVPLSTDDPVIDRLYLSSHRGVIADNQAKWAVPTTRTDDKLRMETCFQQACKGKIQALCENPEWAPLKDNRIPSYGVLSVNLSLTVELKIKIASGFGPLITHGSGMDLYKSNHNNVYWLTIPPMKNLALGVINTLEWIPRFKVSPNLFTVPIKEAGEDCHAPTYLPAEVDGDVKLSSNLVILPGQDLQYVLATYDTSRVEHAVVYYVYSPGRSFSYFYPFRLPIKGVPIELQVECFTWDQKLWCRHFCVLADSESGGHITHSGMVGMGVSCTVTREDGTNTR

SEQ ID NO: 31 - DNA, 1725 bp, Homo sapiens
atggagttgctgatccacaggtcaagtgcaatcttcctaactcttgctgttaatgcattgtacctcacctcaagtcagaacataactgaggagttttaccaatcgacatgtagtgcagttagcagaggttattttagtgctttaagaacaggttggtataccagtgtcataacaatagaattaagtaatataaaagaaaccaaatgcaatggaactgacactaaagtaaaacttataaaacaagaattagataagtataagaatgcagtaacagaattacagctacttatgcaaaacacgccagctgccaacaaccgggccagaagagaagcaccacagtacatgaactacacaatcaataccacaaaaaacctaaatgtatcaataagcaagaaaaggaaacgaagatttctgggcttcttgttaggtgtaggatctgcaatagcaagtggtatagctgtatccaaagttttacaccttgaaggagaagtgaacaaaatcaaaaatgctttgttgtctacaaacaaagctgtagtcagtctatcaaatggggtcagtgttttaaccagcaaagtgttagatctcaagaattacataaataaccgattattacccatagtaaatcaacagagctgtcgcatctccaacattgaaacagttatagaattccagcagatgaatagcagattgttggaaatcaccagagaatttagtgttaatgcaggtgtaacaacacctttaagcacttacatgttaacaaacagtgagttactatcattgatcaatgatatgcctataacaaatgaccagaaaaaattaatgtcaagcaatgttcagatagtaaggcaacaaagttattctatcatgtctataataaaggaagaagtccttgcatatgttgtacagctacctatctatggtgtaatagatacaccttgctggaaattacacacatcacctctatgcaccaccaacatcaaagaaggatcaaatatttgtttaacaaggactgatagaggatggtattgtgataatgcaggatcagtatccttcttcccacaggctgatacttgcaaagtacagtccaatcgagtattttgtgacactatgaacagtttaacattaccaagtgaagtcagcctttgtaacactgacatattcaattccaagtatgactgcaaaattatgacatcaaaaacagacataagcagctcagtaattacttctcttggagctatagtgtcatgctatggaaaaactaaatgcactgcatccaataaaaatcgtgggattataaagacattttctaatggttgtgactatgtgtcaaacaaaggagtagatactgtgtcagtgggcaacactttatactatgtaaacaagctggaaggcaaaaacctttatgtaaaaggggaacctataataaattactatgatcctctagtgtttccttctgatgagtttgatgcatcaatatctcaagtcaatgaaaaaatcaatcaaagtttagcttttattcgtagatctgatgaattactacataatgtaaatactggcaaatctactacaaatattatgataactacaattattatagtaatcattgtagtattgttatcattaatagctattggtttactgttgtattgcaaagccaaaaacacaccagttacactaagcaaagaccaactaagtggaatcaataatattgcattcagcaaatag

SEQ ID NO: 32 - protein, 574 aa, Homo sapiens
MELLIHRSSAIFLTLAVNALYLTSSQNITEEFYQSTCSAVSRGYFSALRTGWYTSVITIELSNIKETKCNGTDTKVKLIKQELDKYKNAVTELQLLMQNTPAANNRARREAPQYMNYTINTTKNLNVSISKKRKRRFLGFLLGVGSAIASGIAVSKVLHLEGEVNKIKNALLSTNKAVVSLSNGVSVLTSKVLDLKNYINNRLLPIVNQQSCRISNIETVIEFQQMNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRRSDELLHNVNTGKSTTNIMITTIIIVIIVVLLSLIAIGLLLYCKAKNTPVTLSKDQLSGINNIAFSK

SEQ ID NO: 33 - DNA, 1497 bp, Escherichia coli
atggcacaagtcattaataccaacagcctctcgctgatcactcaaaataatatcaacaagaaccagtctgcgctgtcgagttctatcgagcgtctgtcttctggcttgcgtattaacagcgcgaaggatgacgcagcgggtcaggcgattgctaaccgtttcacctctaacattaaaggcctgactcaggcggcccgtaacgccaacgacggtatctccgttgcgcagaccaccgaaggcgcgctgtccgaaatcaacaacaacttacagcgtgtgcgtgaactgacggtacaggccactaccggtactaactctgagtctgatctgtcttctatccaggacgaaattaaatcccgtctggatgaaattgaccgcgtatctggtcagacccagttcaacggcgtgaacgtgctggcaaaaaatggctccatgaaaatccaggttggcgcaaatgataaccagactatcactatcgatctgaagcagattgatgctaaaactcttggccttgatggttttagcgttaaaaataacgatacagttaccactagtgctccagtaactgcttttggtgctaccaccacaaacaatattaaacttactggaattaccctttctacggaagcagccactgatactggcggaactaacccagcttcaattgagggtgtttatactgataatggtaatgattactatgcgaaaatcaccggtggtgataacgatgggaagtattacgcagtaacagttgctaatgatggtacagtgacaatggcgactggagcaacggcaaatgcaactgtaactgatgcaaatactactaaagctacaactatcacttcaggcggtacacctgttcagattgataatactgcaggttccgcaactgccaaccttggtgctgttagcttagtaaaactgcaggattccaagggtaatgataccgatacatatgcgcttaaagatacaaatggcaatctttacgctgcggatgtgaatgaaactactggtgctgtttctgttaaaactattacctatactgactcttccggtgccgccagttctccaaccgcggtcaaactgggcggagatgatggcaaaacagaagtggtcgatattgatggtaaaacatacgattctgccgatttaaatggcggtaatctgcaaacaggtttgactgctggtggtgaggctctgactgctgttgcaaatggtaaaaccacggatccgctgaaagcgctggacgatgctatcgcatctgtagacaaattccgttcttccctcggtgcggtgcaaaaccgtctggattccgcggttaccaacctgaacaacaccactaccaacctgtctgaagcgcagtcccgtattcaggacgccgactatgcgaccgaagtgtccaatatgtcgaaagcgcagatcatccagcaggccggtaactccgtgttggcaaaagctaaccaggtaccgcagcaggttctgtctctgctgcagggttaa

SEQ ID NO: 34 - protein, 498 aa, Escherichia coli
MAQVINTNSLSLITQNNINKNQSALSSSIERLSSGLRINSAKDDAAGQAIANRFTSNIKGLTQAARNANDGISVAQTTEGALSEINNNLQRVRELTVQATTGTNSESDLSSIQDEIKSRLDEIDRVSGQTQFNGVNVLAKNGSMKIQVGANDNQTITIDLKQIDAKTLGLDGFSVKNNDTVTTSAPVTAFGATTTNNIKLTGITLSTEAATDTGGTNPASIEGVYTDNGNDYYAKITGGDNDGKYYAVTVANDGTVTMATGATANATVTDANTTKATTITSGGTPVQIDNTAGSATANLGAVSLVKLQDSKGNDTDTYALKDTNGNLYAADVNETTGAVSVKTITYTDSSGAASSPTAVKLGGDDGKTEVVDIDGKTYDSADLNGGNLQTGLTAGGEALTAVANGKTTDPLKALDDAIASVDKFRSSLGAVQNRLDSAVTNLNNTTTNLSEAQSRIQDADYATEVSNMSKAQIIQQAGNSVLAKANQVPQQVLSLLQG

SEQ ID NO: 35 - DNA, 51 bp, artificial
  S-Tag nucleotide sequence
atgaaagaaaccgctgctgctaaattcgaacgccagcacatggacagccca

SEQ ID NO: 36 - protein, 17 aa, artificial
  S-Tag polypeptide
MKETAAAKFERQHMDSP

SEQ ID NO: 37 - DNA, 20 bp, artificial
  primer
ggagctgtcgtattccagtc

SEQ ID NO: 38 - DNA, 20 bp, artificial
  primer
aacccctcaagacccgttta

SEQ ID NO: 39 - protein, 459 aa, Listeria monocytogenes
PIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVVDQNATTHAVKSGDTIWALSVKYGVSVQDIMSWNNLSSSSIYVGQKLAIKQTANTATPKAEVKTEAPAAEKQAAPVVKENTNTNTATTEKKETATQQQTAPKAPTEAAKPAPAPSTNTNANKTNTNT

SEQ ID NO: 40 - protein, 14 aa, artificial
  V5 epitope
GKPIPNPLLGLDST

SEQ ID NO: 41 - DNA, 47 bp, artificial
  primer
gaattgtaatacgactcactatagggaggtgatgaagataccccacc

SEQ ID NO: 42 - DNA, 41 bp, artificial
  primer
taatacgactcactatagggcgaagtgtatcaacaagctgg

SEQ ID NO: 43 - DNA, 47 bp, artificial; identical to SEQ ID NO: 41
  primer

SEQ ID NO: 44 - DNA, 41 bp, artificial; identical to SEQ ID NO: 42
  primer

SEQ ID NO: 45 - DNA, 20 bp, artificial
  primer
ggagctgtcgtattccagtc

SEQ ID NO: 46 - DNA, 20 bp, artificial
  primer
aacccctcaagacccgttta

SEQ ID NO: 47 - DNA, 37 bp, artificial
  primer
cttaaagaattcccaatcgaaaagaaacacgcggatg

SEQ ID NO: 48 - DNA, 30 bp, artificial
  primer
ttctactaattccgagttcgcttttacgag

SEQ ID NO: 49 - DNA, 30 bp, artificial
  primer
ctcgtaaaagcgaactcggaattagtagaa

SEQ ID NO: 50 - DNA, 37 bp, artificial
  primer
agaggtctcgagtgtatttgttttattagcatttgtg

SEQ ID NO: 51 - protein, 479 aa, Listeria monocytogenes
KGGVGPVRRSSRLRRTTQPGPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVVDQNATTHAVKSGDTIWALSVKYGVSVQDIMSWNNLSSSSIYVGQKLAIKQTANTATPKAEVKTEAPAAEKQAAPVVKENTNTNTATTEKKETATQQQTAPKAPTEAAKPAPAPSTNTNANKTNTNT

SEQ ID NO: 52 - protein, 482 aa, Listeria monocytogenes
GRRGLCRGCRTRGRIKQLQSAHKPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVVDQNATTHAVKSGDTIWALSVKYGVSVQDIMSWNNLSSSSIYVGQKLAIKQTANTATPKAEVKTEAPAAEKQAAPVVKENTNTNTATTEKKETATQQQTAPKAPTEAAKPAPAPSTNTNANKTNTNT

SEQ ID NO: 53 - protein, 481 aa, Listeria monocytogenes
RWGYHLRDRKYKGVRSHKGVPRPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVVDQNATTHAVKSGDTIWALSVKYGVSVQDIMSWNNLSSSSIYVGQKLAIKQTANTATPKAEVKTEAPAAEKQAAPVVKENTNTNTATTEKKETATQQQTAPKAPTEAAKPAPAPSTNTNANKTNTNT

SEQ ID NO: 54 - protein, 5 aa, artificial
  synthetic
NPPTT

SEQ ID NO: 55 - protein, 5 aa, artificial
  synthetic
MRRIL

SEQ ID NO: 56 - protein, 4 aa, artificial
  synthetic
MISS

SEQ ID NO: 57 - protein, 5 aa, artificial
  synthetic
RGGSK

SEQ ID NO: 58 - protein, 4 aa, artificial
  synthetic
RGGF

SEQ ID NO: 59 - protein, 5 aa, artificial
  synthetic
NRTVF

SEQ ID NO: 60 - protein, 5 aa, artificial
  synthetic
NRFGL

SEQ ID NO: 61 - protein, 5 aa, artificial
  synthetic
SRHGR

SEQ ID NO: 62 - protein, 5 aa, artificial
  synthetic
IMRHP

SEQ ID NO: 63 - protein, 5 aa, artificial
  synthetic
EVCAP

SEQ ID NO: 64 - protein, 5 aa, artificial
  synthetic
ACGVY

SEQ ID NO: 65 - protein, 5 aa, artificial
  synthetic
CGPKL

SEQ ID NO: 66 - protein, 5 aa, artificial
  synthetic
AGCFS

SEQ ID NO: 67 - protein, 5 aa, artificial
  synthetic
SGGLF

SEQ ID NO: 68 - protein, 5 aa, artificial
  synthetic
AVRLS

SEQ ID NO: 69 - protein, 5 aa, artificial
  synthetic
GGKLS

SEQ ID NO: 70 - protein, 5 aa, artificial
  synthetic
VSEGV

SEQ ID NO: 71 - protein, 5 aa, artificial
  synthetic
KCQSF

SEQ ID NO: 72 - protein, 5 aa, artificial
  synthetic
FCGLG

SEQ ID NO: 73 - protein, 5 aa, artificial
  synthetic
PESGV

SEQ ID NO: 74 - DNA, 49 bp, artificial
  primer
gacgacgacaagattgagaccactcagaacaaggccgcacttaccgacc

SEQ ID NO: 75 - DNA, 39 bp, artificial
  primer
gaggagaagcccggtctattactcgagtgcggccgcaag

SEQ ID NO: 76 - DNA, 57 bp, artificial
  primer
tatgcatcatcaccatcaccatgatgacgacgacaagagcccgggcttctcctcagc

SEQ ID NO: 77 - DNA, 58 bp, artificial
  primer
tcagctgaggagaagcccgggctcttgtcgtcgtcatcatggtgatggtgatgatgca

SEQ ID NO: 78 - DNA, 57 bp, artificial
  primer
gatctcatcatcaccatcaccatgatgacgacgacaagagcccgggcttctcctcaa

SEQ ID NO: 79 - DNA, 57 bp, artificial
  primer
cgcgttgaggagaagcccgggctcttgtcgtcgtcatcatggtgatggtgatgatga

SEQ ID NO: 80 - DNA, 3866 bp, Mus musculus
ctggttgcagaaaatgccaggatgatgcctccctggctcctggctaggactctgatcatggcactgttcttctcctgcctgacaccaggaagcttgaatccctgcatagaggtagttcctaatattacctaccaatgcatggatcagaaactcagcaaagtccctgatgacattccttcttcaaccaagaacatagatctgagcttcaaccccttgaagatcttaaaaagctatagcttctccaatttttcagaacttcagtggctggatttatccaggtgtgaaattgaaacaattgaagacaaggcatggcatggcttacaccacctctcaaacttgatactgacaggaaaccctatccagagtttttccccaggaagtttctctggactaacaagtttagagaatctggtggctgtggagacaaaattggcctctctagaaagcttccctattggacagcttataaccttaaagaaactcaatgtggctcacaattttatacattcctgtaagttacctgcatatttttccaatctgacgaacctagtacatgtggatctttcttataactatattcaaactattactgtcaacgacttacagtttctacgtgaaaatccacaagtcaatctctctttagacatgtctttgaacccaattgacttcattcaagaccaagcctttcagggaattaagctccatgaactgactctaagaggtaattttaatagctcaaatataatgaaaacttgccttcaaaacctggctggtttacacgtccatcggttgatcttgggagaatttaaagatgaaaggaatctggaaatttttgaaccctctatcatggaaggactatgtgatgtgaccattgatgagttcaggttaacatatacaaatgatttttcagatgatattgttaagttccattgcttggcgaatgtttctgcaatgtctctggcaggtgtatctataaaatatctagaagatgttcctaaacatttcaaatggcaatccttatcaatcattagatgtcaacttaagcagtttccaactctggatctaccctttcttaaaagtttgactttaactatgaacaaagggtctatcagttttaaaaaagtggccctaccaagtctcagctatctagatcttagtagaaatgcactgagctttagtggttgctgttcttattctgatttgggaacaaacagcctgagacacttagacctcagcttcaatggtgccatcattatgagtgccaatttcatgggtctagaagagctgcagcacctggattttcagcactctactttaaaaagggtcacagaattctcagcgttcttatcccttgaaaagctactttaccttgacatctcttatactaacaccaaaattgacttcgatggtatatttcttggcttgaccagtctcaacacattaaaaatggctggcaattctttcaaagacaacaccctttcaaatgtctttgcaaacacaacaaacttgacattcctggatctttctaaatgtcaattggaacaaatatcttggggggtatttgacaccctccatagacttcaattattaaatatgagtcacaacaatctattgtttttggattcatcccattataaccagctgtattccctcagcactcttgattgcagtttcaatcgcatagagacatctaaaggaatactgcaacattttccaaagagtctagccttcttcaatcttactaacaattctgttgcttgtatatgtgaacatcagaaattcctgcagtgggtcaaggaacagaagcagttcttggtgaatgttgaacaaatgacatgtgcaacacctgtagagatgaatacctccttagtgttggattttaataattctacctgttatatgtacaagacaatcatcagtgtgtcagtggtcagtgtgattgtggtatccactgtagcatttctgatataccacttctattttcacctgatacttattgctggctgtaaaaagtacagcagaggagaaagcatctatgatgcatttgtgatctactcgagtcagaatgaggactgggtgagaaatgagctggtaaagaatttagaagaaggagtgccccgctttcacctctgccttcactacagagactttattcctggtgtagccattgctgccaacatcatccaggaaggcttccacaagagccggaaggttattgtggtagtgtctagacactttattcagagccgttggtgtatctttgaatatgagattgctcaaacatggcagtttctgagcagccgctctggcatcatcttcattgtccttgagaaggttgagaagtccctgctgaggcagcaggtggaattgtatcgccttcttagcagaaacacctacctggaatgggaggacaatcctctggggaggcacatcttctggagaagacttaaaaatgccctattggatggaaaagcctcgaatcctgagcaaacagcagaggaagaacaagaaacggcaacttggacctgaggagaacaaaactctggggcctaaacccagtctgtttgcaattaataaatgctacagctcacctggggctctgctatggaccgagagcccatggaacacatggctgctaagctatagcatggaccttaccgggcagaaggaagtagcactgacaccttcctttccaggggtatgaattacctaactcgggaaaagaaacataatccagaatctttacctttaatctgaaggagaagaggctaaggcctagtgagaacagaaaggagaaccagtcttcactgggccttttgaatacaagccatgtcatgttctgtgtttcagttgctttagaagagtattgatagtttcaactgaactgaacggtttcttactttcccttttttctactgaatgcaatattaaatagctctttttgagaggtcttcattccaatttcatcttccattttatgtcattttcttttcttttttgtttttatctaattctataagaaatatgattgatacacgctcacagatagcctggccaatcctaagaatgctatatttattaaatacaattcctagtatacttttacttttataaattcagttatcgtttttcatgccttgactataaactaatatcataaataagattgttacaggtatgctaagaaggcccatatttgactataattttttaagaaagtatataaaatatactttgtcatattgtcactgaatgtcattcttaagttattacctaagttatggatgtcacagagtcagtgttaaaaataatttggttgatagaaatatttttaatcaggagggaaaagtggagaggggtgcaggaacagaaatcatgatttcatcatttattcttgatttttccggaagttcacatagctgaatgacaagactacatatgctgcaactgatgttccttctcatcaaggatactctctgaacttgagaacattttggggaggaagaaaggtctaacatccttttccttcatcattctcatttctggacatgccttgtgagatggatcaatgttgggagtacacatttctgctttcaccttatttcagtcagcatgaacactgaatatataatgtcatttcacagtgtgtgtgtgttgtgtatgtacatatatgaacctgtacatgtgtttaagtttaaagagaaaatagtgtacagagcaggtgtatatttgtgatagggctttaaatagttgagctaattcagaaaagtatggaggtttcttggtaaaccaaaccaaaagtagaatcattacaagatctaacaataaaaattttgaaaaaaaaaaaaaaaaaaaaaaaaaaaaaa

SEQ ID NO: 81 - protein, 835 aa, Mus musculus
MMPPWLLARTLIMALFFSCLTPGSLNPCIEVVPNITYQCMDQKLSKVPDDIPSSTKNIDLSFNPLKILKSYSFSNFSELQWLDLSRCEIETIEDKAWHGLHHLSNLILTGNPIQSFSPGSFSGLTSLENLVAVETKLASLESFPIGQLITLKKLNVAHNFIHSCKLPAYFSNLTNLVHVDLSYNYIQTITVNDLQFLRENPQVNLSLDMSLNPIDFIQDQAFQGIKLHELTLRGNFNSSNIMKTCLQNLAGLHVHRLILGEFKDERNLEIFEPSIMEGLCDVTIDEFRLTYTNDFSDDIVKFHCLANVSAMSLAGVSIKYLEDVPKHFKWQSLSIIRCQLKQFPTLDLPFLKSLTLTMNKGSISFKKVALPSLSYLDLSRNALSFSGCCSYSDLGTNSLRHLDLSFNGAIIMSANFMGLEELQHLDFQHSTLKRVTEFSAFLSLEKLLYLDISYTNTKIDFDGIFLGLTSLNTLKMAGNSFKDNTLSNVFANTTNLTFLDLSKCQLEQISWGVFDTLHRLQLLNMSHNNLLFLDSSHYNQLYSLSTLDCSFNRIETSKGILQHFPKSLAFFNLTNNSVACICEHQKFLQWVKEQKQFLVNVEQMTCATPVEMNTSLVLDFNNSTCYMYKTIISVSVVSVIVVSTVAFLIYHFYFHLILIAGCKKYSRGESIYDAFVIYSSQNEDWVRNELVKNLEEGVPRFHLCLHYRDFIPGVAIAANIIQEGFHKSRKVIVVVSRHFIQSRWCIFEYEIAQTWQFLSSRSGIIFIVLEKVEKSLLRQQVELYRLLSRNTYLEWEDNPLGRHIFWRRLKNALLDGKASNPEQTAEEEQETATWT

SEQ ID NO: 82 - DNA, 3814 bp, Homo sapiens
cctctcaccctttagcccagaactgctttgaatacaccaattgctgtggggcggctcgaggaagagaagacaccagtgcctcagaaactgctcggtcagacggtgatagcgagccacgcattcacagggccactgctgctcacagaagcagtgaggatgatgccaggatgatgtctgcctcgcgcctggctgggactctgatcccagccatggccttcctctcctgcgtgagaccagaaagctgggagccctgcgtggaggtggttcctaatattacttatcaatgcatggagctgaatttctacaaaatccccgacaacctccccttctcaaccaagaacctggacctgagctttaatcccctgaggcatttaggcagctatagcttcttcagtttcccagaactgcaggtgctggatttatccaggtgtgaaatccagacaattgaagatggggcatatcagagcctaagccacctctctaccttaatattgacaggaaaccccatccagagtttagccctgggagccttttctggactatcaagtttacagaagctggtggctgtggagacaaatctagcatctctagagaacttccccattggacatctcaaaactttgaaagaacttaatgtggctcacaatcttatccaatctttcaaattacctgagtatttttctaatctgaccaatctagagcacttggacctttccagcaacaagattcaaagtatttattgcacagacttgcgggttctacatcaaatgcccctactcaatctctctttagacctgtccctgaaccctatgaactttatccaaccaggtgcatttaaagaaattaggcttcataagctgactttaagaaataattttgatagtttaaatgtaatgaaaacttgtattcaaggtctggctggtttagaagtccatcgtttggttctgggagaatttagaaatgaaggaaacttggaaaagtttgacaaatctgctctagagggcctgtgcaatttgaccattgaagaattccgattagcatacttagactactacctcgatgatattattgacttatttaattgtttgacaaatgtttcttcattttccctggtgagtgtgactattgaaagggtaaaagacttttcttataatttcggatggcaacatttagaattagttaactgtaaatttggacagtttcccacattgaaactcaaatctctcaaaaggcttactttcacttccaacaaaggtgggaatgctttttcagaagttgatctaccaagccttgagtttctagatctcagtagaaatggcttgagtttcaaaggttgctgttctcaaagtgattttgggacaaccagcctaaagtatttagatctgagcttcaatggtgttattaccatgagttcaaacttcttgggcttagaacaactagaacatctggatttccagcattccaatttgaaacaaatgagtgagttttcagtattcctatcactcagaaacctcatttaccttgacatttctcatactcacaccagagttgctttcaatggcatcttcaatggcttgtccagtctcgaagtcttgaaaatggctggcaattctttccaggaaaacttccttccagatatcttcacagagctgagaaacttgaccttcctggacctctctcagtgtcaactggagcagttgtctccaacagcatttaactcactctccagtcttcaggtactaaatatgagccacaacaacttcttttcattggatacgtttccttataagtgtctgaactccctccaggttcttgattacagtctcaatcacataatgacttccaaaaaacaggaactacagcattttccaagtagtctagctttcttaaatcttactcagaatgactttgcttgtacttgtgaacaccagagtttcctgcaatggatcaaggaccagaggcagctcttggtggaagttgaacgaatggaatgtgcaacaccttcagataagcagggcatgcctgtgctgagtttgaatatcacctgtcagatgaataagaccatcattggtgtgtcggtcctcagtgtgcttgtagtatctgttgtagcagttctggtctataagttctattttcacctgatgcttcttgctggctgcataaagtatggtagaggtgaaaacatctatgatgcctttgttatctactcaagccaggatgaggactgggtaaggaatgagctagtaaagaatttagaagaaggggtgcctccatttcagctctgccttcactacagagactttattcccggtgtggccattgctgccaacatcatccatgaaggtttccataaaagccgaaaggtgattgttgtggtgtcccagcacttcatccagagccgctggtgtatctttgaatatgagattgctcagacctggcagtttctgagcagtcgtgctggtatcatcttcattgtcctgcagaaggtggagaagaccctgctcaggcagcaggtggagctgtaccgccttctcagcaggaacacttacctggagtgggaggacagtgtcctggggcggcacatcttctggagacgactcagaaaagccctgctggatggtaaatcatggaatccagaaggaacagtgggtacaggatgcaattggcaggaagcaacatctatctgaagaggaaaaataaaaacctcctgaggcatttcttgcccagctgggtccaacacttgttcagttaataagtattaaatgctgccacatgtcaggccttatgctaagggtgagtaattccatggtgcactagatatgcagggctgctaatctcaaggagcttccagtgcagagggaataaatgctagactaaaatacagagtcttccaggtgggcatttcaaccaactcagtcaaggaacccatgacaaagaaagtcatttcaactcttacctcatcaagttgaataaagacagagaaaacagaaagagacattgttcttttcctgagtcttttgaatggaaattgtattatgttatagccatcataaaaccattttggtagttttgactgaactgggtgttcactttttcctttttgattgaatacaatttaaattctacttgatgactgcagtcgtcaaggggctcctgatgcaagatgccccttccattttaagtctgtctccttacagaggttaaagtctagtggctaattcctaaggaaacctgattaacacatgctcacaaccatcctggtcattctcgagcatgttctattttttaactaatcacccctgatatatttttatttttatatatccagttttcatttttttacgtcttgcctataagctaatatcataaataaggttgtttaagacgtgcttcaaatatccatattaaccactatttttcaaggaagtatggaaaagtacactctgtcactttgtcactcgatgtcattccaaagttattgcctactaagtaatgactgtcatgaaagcagcattgaaataatttgtttaaagggggcactcttttaaacgggaagaaaatttccgcttcctggtcttatcatggacaatttgggctagaggcaggaaggaagtgggatgacctcaggaggtcaccttttcttgattccagaaacatatgggctgataaacccggggtgacctcatgaaatgagttgcagcagaagtttatttttttcagaacaagtgatgtttgatggacctctgaatctctttagggagacacagatggctgggatccctcccctgtacccttctcactgccaggagaacta

SEQ ID NO: 83 - protein, 839 aa, Homo sapiens
MMSASRLAGTLIPAMAFLSCVRPESWEPCVEVVPNITYQCMELNFYKIPDNLPFSTKNLDLSFNPLRHLGSYSFFSFPELQVLDLSRCEIQTIEDGAYQSLSHLSTLILTGNPIQSLALGAFSGLSSLQKLVAVETNLASLENFPIGHLKTLKELNVAHNLIQSFKLPEYFSNLTNLEHLDLSSNKIQSIYCTDLRVLHQMPLLNLSLDLSLNPMNFIQPGAFKEIRLHKLTLRNNFDSLNVMKTCIQGLAGLEVHRLVLGEFRNEGNLEKFDKSALEGLCNLTIEEFRLAYLDYYLDDIIDLFNCLTNVSSFSLVSVTIERVKDFSYNFGWQHLELVNCKFGQFPTLKLKSLKRLTFTSNKGGNAFSEVDLPSLEFLDLSRNGLSFKGCCSQSDFGTTSLKYLDLSFNGVITMSSNFLGLEQLEHLDFQHSNLKQMSEFSVFLSLRNLIYLDISHTHTRVAFNGIFNGLSSLEVLKMAGNSFQENFLPDIFTELRNLTFLDLSQCQLEQLSPTAFNSLSSLQVLNMSHNNFFSLDTFPYKCLNSLQVLDYSLNHIMTSKKQELQHFPSSLAFLNLTQNDFACTCEHQSFLQWIKDQRQLLVEVERMECATPSDKQGMPVLSLNITCQMNKTIIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 84 - DNA, 3934 bp, Homo sapiens
cctctcaccctttagcccagaactgctttgaatacaccaattgctgtggggcggctcgaggaagagaagacaccagtgcctcagaaactgctcggtcagacggtgatagcgagccacgcattcacagggccactgctgctcacagaagcagtgaggatgatgccaggatgatgtctgcctcgcgcctggctgggactctgatcccagccatggccttcctctcctgcgtgagaccagaaagctgggagccctgcgtggagacttggccctaaaccacacagaagagctggcatgaaacccagagctttcagactccggagcctcagcccttcaccccgattccattgcttcttgctaaatgctgccgttttatcacggaggtggttcctaatattacttatcaatgcatggagctgaatttctacaaaatccccgacaacctccccttctcaaccaagaacctggacctgagctttaatcccctgaggcatttaggcagctatagcttcttcagtttcccagaactgcaggtgctggatttatccaggtgtgaaatccagacaattgaagatggggcatatcagagcctaagccacctctctaccttaatattgacaggaaaccccatccagagtttagccctgggagccttttctggactatcaagtttacagaagctggtggctgtggagacaaatctagcatctctagagaacttccccattggacatctcaaaactttgaaagaacttaatgtggctcacaatcttatccaatctttcaaattacctgagtatttttctaatctgaccaatctagagcacttggacctttccagcaacaagattcaaagtatttattgcacagacttgcgggttctacatcaaatgcccctactcaatctctctttagacctgtccctgaaccctatgaactttatccaaccaggtgcatttaaagaaattaggcttcataagctgactttaagaaataattttgatagtttaaatgtaatgaaaacttgtattcaaggtctggctggtttagaagtccatcgtttggttctgggagaatttagaaatgaaggaaacttggaaaagtttgacaaatctgctctagagggcctgtgcaatttgaccattgaagaattccgattagcatacttagactactacctcgatgatattattgacttatttaattgtttgacaaatgtttcttcattttccctggtgagtgtgactattgaaagggtaaaagacttttcttataatttcggatggcaacatttagaattagttaactgtaaatttggacagtttcccacattgaaactcaaatctctcaaaaggcttactttcacttccaacaaaggtgggaatgctttttcagaagttgatctaccaagccttgagtttctagatctcagtagaaatggcttgagtttcaaaggttgctgttctcaaagtgattttgggacaaccagcctaaagtatttagatctgagcttcaatggtgttattaccatgagttcaaacttcttgggcttagaacaactagaacatctggatttccagcattccaatttgaaacaaatgagtgagttttcagtattcctatcactcagaaacctcatttaccttgacatttctcatactcacaccagagttgctttcaatggcatcttcaatggcttgtccagtctcgaagtcttgaaaatggctggcaattctttccaggaaaacttccttccagatatcttcacagagctgagaaacttgaccttcctggacctctctcagtgtcaactggagcagttgtctccaacagcatttaactcactctccagtcttcaggtactaaatatgagccacaacaacttcttttcattggatacgtttccttataagtgtctgaactccctccaggttcttgattacagtctcaatcacataatgacttccaaaaaacaggaactacagcattttccaagtagtctagctttcttaaatcttactcagaatgactttgcttgtacttgtgaacaccagagtttcctgcaatggatcaaggaccagaggcagctcttggtggaagttgaacgaatggaatgtgcaacaccttcagataagcagggcatgcctgtgctgagtttgaatatcacctgtcagatgaataagaccatcattggtgtgtcggtcctcagtgtgcttgtagtatctgttgtagcagttctggtctataagttctattttcacctgatgcttcttgctggctgcataaagtatggtagaggtgaaaacatctatgatgcctttgttatctactcaagccaggatgaggactgggtaaggaatgagctagtaaagaatttagaagaaggggtgcctccatttcagctctgccttcactacagagactttattcccggtgtggccattgctgccaacatcatccatgaaggtttccataaaagccgaaaggtgattgttgtggtgtcccagcacttcatccagagccgctggtgtatctttgaatatgagattgctcagacctggcagtttctgagcagtcgtgctggtatcatcttcattgtcctgcagaaggtggagaagaccctgctcaggcagcaggtggagctgtaccgccttctcagcaggaacacttacctggagtgggaggacagtgtcctggggcggcacatcttctggagacgactcagaaaagccctgctggatggtaaatcatggaatccagaaggaacagtgggtacaggatgcaattggcaggaagcaacatctatctgaagaggaaaaataaaaacctcctgaggcatttcttgcccagctgggtccaacacttgttcagttaataagtattaaatgctgccacatgtcaggccttatgctaagggtgagtaattccatggtgcactagatatgcagggctgctaatctcaaggagcttccagtgcagagggaataaatgctagactaaaatacagagtcttccaggtgggcatttcaaccaactcagtcaaggaacccatgacaaagaaagtcatttcaactcttacctcatcaagttgaataaagacagagaaaacagaaagagacattgttcttttcctgagtcttttgaatggaaattgtattatgttatagccatcataaaaccattttggtagttttgactgaactgggtgttcactttttcctttttgattgaatacaatttaaattctacttgatgactgcagtcgtcaaggggctcctgatgcaagatgccccttccattttaagtctgtctccttacagaggttaaagtctagtggctaattcctaaggaaacctgattaacacatgctcacaaccatcctggtcattctcgagcatgttctattttttaactaatcacccctgatatatttttatttttatatatccagttttcatttttttacgtcttgcctataagctaatatcataaataaggttgtttaagacgtgcttcaaatatccatattaaccactatttttcaaggaagtatggaaaagtacactctgtcactttgtcactcgatgtcattccaaagttattgcctactaagtaatgactgtcatgaaagcagcattgaaataatttgtttaaagggggcactcttttaaacgggaagaaaatttccgcttcctggtcttatcatggacaatttgggctagaggcaggaaggaagtgggatgacctcaggaggtcaccttttcttgattccagaaacatatgggctgataaacccggggtgacctcatgaaatgagttgcagcagaagtttatttttttcagaacaagtgatgtttgatggacctctgaatctctttagggagacacagatggctgggatccctcccctgtacccttctcactgccaggagaacta

SEQ ID NO: 85 - protein, 799 aa, Homo sapiens
MELNFYKIPDNLPFSTKNLDLSFNPLRHLGSYSFFSFPELQVLDLSRCEIQTIEDGAYQSLSHLSTLILTGNPIQSLALGAFSGLSSLQKLVAVETNLASLENFPIGHLKTLKELNVAHNLIQSFKLPEYFSNLTNLEHLDLSSNKIQSIYCTDLRVLHQMPLLNLSLDLSLNPMNFIQPGAFKEIRLHKLTLRNNFDSLNVMKTCIQGLAGLEVHRLVLGEFRNEGNLEKFDKSALEGLCNLTIEEFRLAYLDYYLDDIIDLFNCLTNVSSFSLVSVTIERVKDFSYNFGWQHLELVNCKFGQFPTLKLKSLKRLTFTSNKGGNAFSEVDLPSLEFLDLSRNGLSFKGCCSQSDFGTTSLKYLDLSFNGVITMSSNFLGLEQLEHLDFQHSNLKQMSEFSVFLSLRNLIYLDISHTHTRVAFNGIFNGLSSLEVLKMAGNSFQENFLPDIFTELRNLTFLDLSQCQLEQLSPTAFNSLSSLQVLNMSHNNFFSLDTFPYKCLNSLQVLDYSLNHIMTSKKQELQHFPSSLAFLNLTQNDFACTCEHQSFLQWIKDQRQLLVEVERMECATPSDKQGMPVLSLNITCQMNKTIIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 86 - DNA, 4286 bp, Mus musculus
ttgaaatctcacagcccggttggttgcagtgacccacttcgttgaacatattcttcctaatcctagtactttcaatttgctctattccctggtgtctatgcatttaaatcgactatggggccattcttccttgaaccaccacagaagacattagctctctgggatccttgttaattttttctcctcttacatagcacctacgcttggaacatatgccagacacatctgtgagacaccccttgccgctgcagctcatggatggatgctgagttcccccacgcaccacacttcagcaggtgggtgtatttctgcttcacattatactcccacacggccatgcatgtcaggcatggagcaggctcataacccacttaattaaggtgatcatatcagatcctttatcaagatgcatagagtgctcagtgcctgtactatgatctcggatctttgggagatgggctagatagagtctgggacagaatacagcagagaaaccgatatgtttattgtccgatcatcagctaagcttctgggagctaggaatggggctccttggatgaacagaagtaaaaatgcctcgtctttatgactttcaacttccctcagcaggtctggaatgggtgaacaaacactgcctgcgtgggtgataaatagcctctttttgctgcttgtttgctgcttttatggttctgggagggaacctagaacctagcacatgctagacaagtcctctagcactgagctatctccccagcttggatgaaatatctgtaaagtactggtgcccgtgtgtaaaatatgcaccattaagtgttcaagaagaaaagactgggcatttctgttccaccaagacaagaagaatctgccagcagaatgtttgcgcagtcatttgagcaaaggggtccaagggacagtaccctccagtgctggggacccatgtgccgagcctcaggctgtgatgtggtgttgtttttaattctctcttttcccataggatcatggcatgtcaacttgacttgctcataggtgtgatcttcatggccagccccgtgttggtaatatctccctgttcttcagacggcaggatagcctttttccgaggctgtaacctcacccagattccctggatcctcaatactaccactgagaggctcctgctcagcttcaactatatcagtatggtggttgccacatcatttccactcctggagcggctccagttgctggagctggggacccagtatgctaacttgaccattggtccaggggctttcagaaacctgcccaatcttaggatcttggacttgggccaaagccagatcgaagtcttgaatcgagatgcctttcaaggtctgccccatctcttggaacttcggctgttttcctgtggactctccagtgctgtgttaagtgacggttacttcagaaatctatattcattagctcgcttagacctatctggcaaccagattcacagcctccgcctccattcttcattccgggaactgaattccttaagcgacgtaaattttgctttcaaccaaatattcactatatgtgaagatgaactcgagcctctgcagggcaaaacactgtctttctttggcctcaaattaactaagctgttcagcagagtctctgtgggctgggagacatgcaggaaccccttcagaggcgtgaggctagaaactctagatctttctgaaaatggctggacggtggacatcacaaggaacttcagcaacatcatccagggaagccagatttcctctttgattcttaaacaccacatcatgggtcctggctttggcttccagaacatcagagatcctgaccagagcacatttgccagcctggccagaagttcggtgctgcaactggacctttcgcacggctttatcttctccttgaatcctcgactgtttgggacactgaaggatttgaagatgctgaaccttgccttcaacaagataaacaagattggagagaatgccttttatgggcttgacagcctccaggttctcaatctatcctataatcttttgggggaactctataattccaacttctatgggcttcctagagtagcctacgttgaccttcaaaggaaccacattgggatcattcaagaccaaacattcagattattaaaaacgttacaaaccttagatctccgtgacaatgctcttaaggccattggttttattccaagcatacagatggtcctcctgggaggcaataagctggtccatttgccacacatccactttactgccaacttcctagagttatctgaaaacaggctagaaaacctgtccgacctctacttcctcctgcgagtcccccagctccagtttctcatcttgaatcagaatcgcctttcgtcatgcaaggcagcccacactccctcggagaacccaagcttagaacagcttttccttacagagaatatgctgcagctggcctgggagaccggcctctgttgggatgtttttcaaggcctttcccgcctccagattctttacctgagtaataactaccttaatttccttccacctgggatatttaacgacctggttgcattacggatgcttagtcttagtgctaacaagctgaccgtgctctctccgggcagtttacctgctaatttagagattctcgacatatctagaaatcagcttttgtgtcctgaccctgctttgttttcttcgcttcgtgttttggacataactcataacgagttcgtctgcaactgtgaacttagcacttttatctcctggctcaaccaaaccaacgtcaccctgttcggctctcctgcagacgtgtattgcatgtaccctaactcactgctagggggctccctctacaacatatccaccgaagactgcgatgaagaggaagccatgcggtccctaaagttttcccttttcatcctgtgcacggtcactttgactctattcctcgtcatcacccttgtagtcataaagttccggggaatctgtttcctgtgctataagaccatccagaagctggtgttcaaggacaaggtctggagtttggaacctggtgcatatagatatgatgcctacttctgcttcagcagcaaagactttgaatgggcacagaatgctttgctcaaacacctggatgctcactacagttcccgaaacaggctcaggctatgctttgaagaaagagacttcattccgggggaaaaccatatctccaacatccaggcggctgtctggggcagcaggaagacggtgtgtctagtgagcagacacttcctgaaggatggttggtgcctggaggccttcaggtatgcccagagccggagtctgtctgacctcaagagcattctcatcgtggtggtggtgggatcgctgtcccagtatcagctgatgagacatgagaccatcagagggtttctgcaaaagcaacagtacttgaggtggcctgaagacctccaggatgttggctggtttctcgataaactctccggatgcattctaaaggaagaaaaaggaaagaaaagaagcagttccatccagttgcgaaccatagcaaccatttcctagcaggagcgcctcctagcagaagtgcaagcatcgtagataactctccacgctttatccgcacagccgctgggggtccttccctggagtcatttttctgacaatgaaaacaacaccaatctcttgatttttcatgtcaacagggagctttgtcttcactgttttccaaatggaaagtaagaggtccagaaagctgcctctaagggctctcacctgccattgatgtcctttcaggcccaatgacatggtttccctccatcctattgcgtactgtctgctacccaggtggcaagagcaccttgggagaagttacaggcagcttcatgctttctgtgctgttcagttcaaaagcaggtgccttgagaatcctgaattcaagcactctgtagaacatggacagacaagatgggtccttctctggccataggcatgagggccagttgctgaggactgctctcactacacctaagtgcacaagtgataagaagttggacagatagacagatagcagcagtcccattgctgtagccagaatgcacttatttcctgttctgaccctgcaggcccagcttttggggaccacagccatgttctgcacgggacctctcaacctggcattcatgccctttcacgacttagcaccggcctgcccttctttcttccccacaactatacaagagctgttgcaaccactgaaaaaaaaaaaaaaaa

SEQ ID NO: 87 - protein, 859 aa, Mus musculus
MACQLDLLIGVIFMASPVLVISPCSSDGRIAFFRGCNLTQIPWILNTTTERLLLSFNYISMVVATSFPLLERLQLLELGTQYANLTIGPGAFRNLPNLRILDLGQSQIEVLNRDAFQGLPHLLELRLFSCGLSSAVLSDGYFRNLYSLARLDLSGNQIHSLRLHSSFRELNSLSDVNFAFNQIFTICEDELEPLQGKTLSFFGLKLTKLFSRVSVGWETCRNPFRGVRLETLDLSENGWTVDITRNFSNIIQGSQISSLILKHHIMGPGFGFQNIRDPDQSTFASLARSSVLQLDLSHGFIFSLNPRLFGTLKDLKMLNLAFNKINKIGENAFYGLDSLQVLNLSYNLLGELYNSNFYGLPRVAYVDLQRNHIGIIQDQTFRLLKTLQTLDLRDNALKAIGFIPSIQMVLLGGNKLVHLPHIHFTANFLELSENRLENLSDLYFLLRVPQLQFLILNQNRLSSCKAAHTPSENPSLEQLFLTENMLQLAWETGLCWDVFQGLSRLQILYLSNNYLNFLPPGIFNDLVALRMLSLSANKLTVLSPGSLPANLEILDISRNQLLCPDPALFSSLRVLDITHNEFVCNCELSTFISWLNQTNVTLFGSPADVYCMYPNSLLGGSLYNISTEDCDEEEAMRSLKFSLFILCTVTLTLFLVITLVVIKFRGICFLCYKTIQKLVFKDKVWSLEPGAYRYDAYFCFSSKDFEWAQNALLKHLDAHYSSRNRLRLCFEERDFIPGENHISNIQAAVWGSRKTVCLVSRHFLKDGWCLEAFRYAQSRSLSDLKSILIVVVVGSLSQYQLMRHETIRGFLQKQQYLRWPEDLQDVGWFLDKLSGCILKEEKGKKRSSSIQLRTIATIS

SEQ ID NO: 88 - DNA, 3369 bp, Homo sapiens
ggttttcaggagcccgagcgagggcgccgcttttgcgtccgggaggagccaaccgtggcgcaggcggcgcggggaggcgtcccagagtctcactctgccgcccaggctggactgcagtgacacaatctcggctgactgcaaccactgcctccagggttcaagcgattctcttgcctcagcctcccaagtagctgggattacagattgatgttcatgttcctggcactactacaagattcatactcctgatgctactgacaacgtggcttctccacagtcaccaaaccagggatgctatactggacttccctactctcatctgctccagccccctgaccttatagttgcccagctttcctggcaattgactttgcccatcaatacacaggatttagcatccagggaagatgtcggagcctcagatgttaattttctaattgagaatgttggcgctgtccgaacctggagacaggaaaacaaaaagtcctttctcctgattcaccaaaaaataaaatactgactaccatcactgtgatgagattcctatagtctcaggaactgaagtctttaaacaaccagggaccctctgcccctagaataagaacatactagaagtcccttctgctaggacaacgaggatcatgggagaccacctggaccttctcctaggagtggtgctcatggccggtcctgtgtttggaattccttcctgctcctttgatggccgaatagccttttatcgtttctgcaacctcacccaggtcccccaggtcctcaacaccactgagaggctcctgctgagcttcaactatatcaggacagtcactgcttcatccttcccctttctggaacagctgcagctgctggagctcgggagccagtatacccccttgactattgacaaggaggccttcagaaacctgcccaaccttagaatcttggacctgggaagtagtaagatatacttcttgcatccagatgcttttcagggactgttccatctgtttgaacttagactgtatttctgtggtctctctgatgctgtattgaaagatggttatttcagaaatttaaaggctttaactcgcttggatctatccaaaaatcagattcgtagcctttaccttcatccttcatttgggaagttgaattccttaaagtccatagatttttcctccaaccaaatattccttgtatgtgaacatgagctcgagcccctacaagggaaaacgctctccttttttagcctcgcagctaatagcttgtatagcagagtctcagtggactggggaaaatgtatgaacccattcagaaacatggtgctggagatactagatgtttctggaaatggctggacagtggacatcacaggaaactttagcaatgccatcagcaaaagccaggccttctctttgattcttgcccaccacatcatgggtgccgggtttggcttccataacatcaaagatcctgaccagaacacatttgctggcctggccagaagttcagtgagacacctggatctttcacatgggtttgtcttctccctgaactcacgagtctttgagacactcaaggatttgaaggttctgaaccttgcctacaacaagataaataagattgcagatgaagcattttacggacttgacaacctccaagttctcaatttgtcatataaccttctgggggaactttacagttcgaatttctatggactacctaaggtagcctacattgatttgcaaaagaatcacattgcaataattcaagaccaaacattcaaattcctggaaaaattacagaccttggatctccgagacaatgctcttacaaccattcattttattccaagcatacccgatatcttcttgagtggcaataaactagtgactttgccaaagatcaaccttacagcgaacctcatccacttatcagaaaacaggctagaaaatctagatattctctactttcttctacgggtacctcatctccagattctcattttaaatcaaaatcgcttctcctcctgtagtggagatcaaaccccttcagagaatcccagcttagaacagcttttccttggagaaaatatgttgcaacttgcctgggaaactgagctctgttgggatgtttttgagggactttctcatcttcaagttctgtatttgaatcataactatcttaattcccttccaccaggagtatttagccatctgactgcattaaggggactaagcctcaactccaacaggctgacagttctttctcacaatgatttacctgctaatttagagatcctggacatatccaggaaccagctcctagctcctaatcctgatgtatttgtatcacttagtgtcttggatataactcataacaagttcatttgtgaatgtgaacttagcacttttatcaattggcttaatcacaccaatgtcactatagctgggcctcctgcagacatatattgtgtgtaccctgactcgttctctggggtttccctcttctctctttccacggaaggttgtgatgaagaggaagtcttaaagtccctaaagttctcccttttcattgtatgcactgtcactctgactctgttcctcatgaccatcctcacagtcacaaagttccggggcttctgttttatctgttataagacagcccagagactggtgttcaaggaccatccccagggcacagaacctgatatgtacaaatatgatgcctatttgtgcttcagcagcaaagacttcacatgggtgcagaatgctttgctcaaacacctggacactcaatacagtgaccaaaacagattcaacctgtgctttgaagaaagagactttgtcccaggagaaaaccgcattgccaatatccaggatgccatctggaacagtagaaagatcgtttgtcttgtgagcagacacttccttagagatggctggtgccttgaagccttcagttatgcccagggcaggtgcttatctgaccttaacagtgctctcatcatggtggtggttgggtccttgtcccagtaccagttgatgaaacatcaatccatcagaggctttgtacagaaacagcagtatttgaggtggcctgaggatctccaggatgttggctggtttcttcataaactctctcaacagatactaaagaaagaaaaagaaaagaagaaagacaataacattccgttgcaaactgtagcaaccatctcctaatcaaaggagcaatttccaacttatctcaagccacaaataactcttcactttgtatttgcaccaagttatcattttggggtcctctctggaggttttttttttctttttgctactatgaaaacaacataaatctctcaattttcgtatcaaa

SEQ ID NO: 89 - protein, 858 aa, Homo sapiens
MGDHLDLLLGVVLMAGPVFGIPSCSFDGRIAFYRFCNLTQVPQVLNTTERLLLSFNYIRTVTASSFPFLEQLQLLELGSQYTPLTIDKEAFRNLPNLRILDLGSSKIYFLHPDAFQGLFHLFELRLYFCGLSDAVLKDGYFRNLKALTRLDLSKNQIRSLYLHPSFGKLNSLKSIDFSSNQIFLVCEHELEPLQGKTLSFFSLAANSLYSRVSVDWGKCMNPFRNMVLEILDVSGNGWTVDITGNFSNAISKSQAFSLILAHHIMGAGFGFHNIKDPDQNTFAGLARSSVRHLDLSHGFVFSLNSRVFETLKDLKVLNLAYNKINKIADEAFYGLDNLQVLNLSYNLLGELYSSNFYGLPKVAYIDLQKNHIAIIQDQTFKFLEKLQTLDLRDNALTTIHFIPSIPDIFLSGNKLVTLPKINLTANLIHLSENRLENLDILYFLLRVPHLQILILNQNRFSSCSGDQTPSENPSLEQLFLGENMLQLAWETELCWDVFEGLSHLQVLYLNHNYLNSLPPGVFSHLTALRGLSLNSNRLTVLSHNDLPANLEILDISRNQLLAPNPDVFVSLSVLDITHNKFICECELSTFINWLNHTNVTIAGPPADIYCVYPDSFSGVSLFSLSTEGCDEEEVLKSLKFSLFIVCTVTLTLFLMTILTVTKFRGFCFICYKTAQRLVFKDHPQGTEPDMYKYDAYLCFSSKDFTWVQNALLKHLDTQYSDQNRFNLCFEERDFVPGENRIANIQDAIWNSRKIVCLVSRHFLRDGWCLEAFSYAQGRCLSDLNSALIMVVVGSLSQYQLMKHQSIRGFVQKQQYLRWPEDLQDVGWFLHKLSQQILKKEKEKKKDNNIPLQTVATIS

SEQ ID NO: 90 - DNA, 76 bp, artificial
  synthetic oligonucleotide
catgcccggaattcctgcnnknnknnknnknnknnknnknnknnknnktgcggaggaggataaaagctttcgagac

SEQ ID NO: 91 - DNA, 67 bp, artificial
  synthetic oligonucleotide
catgcccggaattcctgcnnknnknnknnknnknnknnktgcggaggaggataaaagctttcgagac